SEQUENCE LISTING (3 sequences)

SEQ ID NO: 1 - RNA, 140 nt, Synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 2 - RNA, 105 nt, Synthetic construct
  source 1..105: mol_type = other RNA
cgggtggcatccctgtgacccctccccagtgcctctcctggccctggaagttgccactccagtgcccaccagccttgtcctaataaaattaagttgcatcaagct

SEQ ID NO: 3 - RNA, 105 nt, Synthetic construct
  source 1..105: mol_type = other RNA
gggtggcatccctgtgacccctccccagtgcctctcctggccctggaagttgccactccagtgcccaccagccttgtcctaataaaattaagttgcatcaaagct

(19) United States
(12) Patent Application Publication
(10) Pub. No.: US 2026/0294818 A1
(43) Pub. Date: Oct. 1, 2026

(54) RECONSTITUTABLE DRY POWDER FORMULATIONS AND METHODS OF USE THEREOF

(71) Applicant: SANOFI PASTEUR INC., Swiftwater, PA (US)

(72) Inventors: Ashish SARODE, Cambridge, MA (US); Shrirang KARVE, Cambridge, MA (US); Priyal PATEL, Cambridge, MA (US)

(21) Appl. No.: 19/475,932

(22) PCT Filed: Apr. 17, 2024
(86) PCT No.: PCT/EP2024/060446

§ 371 Date: Oct. 16, 2025

(30) Foreign Application Priority Data
Apr. 17, 2023 (EP) 23305580.5

(51) Int. Cl. A61K 9/50; A61K 9/10; A61K 48/00

(52) U.S. Cl. CPC A61K 9/5015; A61K 9/10; A61K 48/0033

(57) ABSTRACT

The present disclosure is directed to the use of reconstituted mRNA dry powder particles for parenteral administration. The present disclosure is also directed to a method of generating dry powder particles supplemented with appropriate excipients for optimal thermostability and in vivo expression.

### BACKGROUND

Messenger RNA therapy (MRT) is an important approach for the treatment of a variety of diseases. Uses of in vitro transcribed mRNA for the production of proteins in animals were first reported in the 1990s. With the advancement in biotechnology, mRNA stability and immunogenicity have been optimized to be useful in a clinical setting in human beings. However, safe and efficient in vivo delivery remains a major obstacle in mRNA therapeutics development.

Dry powder formulations of mRNA can be useful and advantageous (including for inhaled delivery to the lung). Reconstitutable dry powder formulations can provide additional benefits, including, for example, for improved formulations useful for other routes of administration such as for parenteral administration. In particular, overcoming the challenges of developing both thermostable as well as reconstitutable dry powder formulations of mRNA-LNPs have presented challenges in the development of dry powder formulations suitable for, e.g., parenteral administration. There remains a need for improved dry powder formulations, including reconstitutable dry powder formulations.

### SUMMARY

Described herein are dry powder formulations that can provide certain benefits and improvements. For example, dry powder formulations provided herein can be reconstitutable and therefore suitable for a variety of applications and provide various therapeutic benefits. Also described herein are methods of making and reconstituting dry powder formulations as well as methods of use of the dry powder formulations.

In some aspects, the present disclosure encompasses a dry powder formulation for reconstitution comprising: (a) an excipient that is sucrose or trehalose; and (b) a lipid nanoparticle (LNP) comprising a messenger RNA (mRNA) encapsulated by one or more lipids; and wherein the weight (w/w) ratio of the excipient of (a) and the total lipids in the LNP of (b) is at least about 5. In some embodiments, the excipient is sucrose. In some embodiments, the excipient is trehalose. In some embodiments, the w/w ratio of the excipient to the total lipids in the LNP is at least about 5.6, 11, or 15. In some embodiments, the w/w ratio of the excipient to the total lipids in the LNP is at least about 11.1 or at least about 15.6. In some embodiments, the LNP comprises one or more cationic lipids, one or more non-cationic lipids, and one or more PEG-modified lipids.

In some aspects, the present disclosure encompasses a dry powder formulation for reconstitution comprising: (a) an excipient that is a sugar or sugar alcohol; and (b) a lipid nanoparticle (LNP) comprising messenger RNA (mRNA) encapsulated by one or more lipids; and wherein the weight (w/w) ratio of the excipient of (a) and total lipids in the LNP of (b) is at least about 5. In some embodiments, the sugar or the sugar alcohol is selected from sucrose, mannitol, xylitol, lactose, and trehalose. In some embodiments, the sugar or the sugar alcohol is trehalose. In some embodiments, the sugar or the sugar alcohol is sucrose. In some embodiments, the w/w ratio of the sugar or the sugar alcohol to the total lipids in the LNP is at least about 5.6, 11, or 15. In some embodiments, the w/w ratio of the sugar or the sugar alcohol to the total lipids in the LNP is at least about 11.1 or at least about 15.6. In some embodiments, the LNP comprises one or more cationic lipids, one or more non-cationic lipids, and one or more PEG-modified lipids.

In some embodiments, the particle size (e.g., mean particle size) of the reconstituted dry powder formulation is less than about 120 nm. In some embodiments, the encapsulation rate is greater than 60%. In some embodiments, the particle size (e.g., mean particle size) of the dry powder is less than 5 μm. In some embodiments, the dry powder formulation is stable after prolonged storage, e.g., after prolonged storage at 2-8° C. (e.g., at 4° C.). In some embodiments, the mRNA maintains an integrity of 80% or greater after storage, e.g., storage at 2-8° C. (e.g., at 4° C.), for at least six months. In some embodiments, the mRNA maintains an integrity of 80% or greater, e.g., after storage at 2-8° C. (e.g., 4° C.), for up to 1 year. In some embodiments, the mRNA maintains an integrity of 80% or greater, e.g., after storage at 2-8° C. (e.g., 4° C.), for at least 1 year. In some embodiments, the mRNA maintains an integrity of about 80% or greater, e.g., after storage at 2-8° C. (e.g., 4° C.), for more than 1 year.

In some embodiments, the mRNA encodes a therapeutic protein. In some embodiments, the mRNA encodes an antigen. In some embodiments, the formulation is suitable for a vaccine. In some embodiments, the formulation is suitable for parenteral delivery once reconstituted. In some embodiments, the formulation is suitable for intramuscular, intravenous, or subcutaneous delivery once reconstituted. In some embodiments, the formulation is suitable for mucosal delivery once reconstituted. In some embodiments, the formulation is suitable for oral, sublingual, or intranasal delivery once reconstituted.

In some aspects, the present disclosure encompasses a method of delivering mRNA in vivo, the method comprising administering to a subject in need thereof a reconstituted form of the dry powder formulation of the present disclosure. In some embodiments, the present disclosure encompasses a method of treating a disease or disorder in a subject, the method comprising administering to the subject a reconstituted form of the dry powder formulation of the present disclosure. In some embodiments, the reconstituted form of the dry powder formulation is administered subcutaneously, intravenously, or intramuscularly.

In some aspects, the present disclosure encompasses a reconstituted form of the dry powder formulation of the present disclosure for use in treating a disease or disorder in a subject. In some aspects, the present disclosure encompasses the use of a reconstituted form of the dry powder formulation of the present disclosure in the manufacture of a medicament for treating a disease or disorder in a subject. In some embodiments, the reconstituted form of the dry powder formulation is formulated for subcutaneous, intravenous, or intramuscular administration.

In some aspects, the present disclosure encompasses a method of preparing a dry powder formulation for reconstitution, the method comprising: (a) combining a first mixture comprising lipid nanoparticles (LNPs) and an ethanolic solution, thereby obtaining a second mixture, wherein the LNPs comprise a messenger RNA (mRNA) encapsulated by one or more lipids, and wherein the ethanolic solution comprises an excipient that is sucrose or trehalose at a concentration of about 1-11% w/v, and (b) spray-drying the second mixture, thereby obtaining the dry powder formulation.

In some embodiments, the present disclosure encompasses a method of preparing a reconstituted dry powder formulation, the method comprising: (a) providing a dry powder formulation prepared according to the present disclosure; and (b) reconstituting the dry powder formulation in water or buffer, thereby obtaining the reconstituted dry powder formulation. In some embodiments, the LNPs in the reconstituted dry powder formulation have a diameter of about 100 nm. In some embodiments, the weight ratio (w/w) of sucrose or trehalose to total lipids in the LNPs is at least about 5, at least about 11, or at least about 15. In some embodiments, the concentration of sucrose or trehalose is greater than 2% (w/v), greater than 5% (w/v), or greater than 7% (w/v) post-reconstitution. In some embodiments, the concentration of sucrose or trehalose is from about 2% (w/v) to about 10% (w/v) post-reconstitution. In some embodiments, the particle size of the reconstituted dry powder formulation (e.g., the mean particle size of the LNP in the reconstituted formulation) is less than 120 nm. In some embodiments, the reconstituted LNP particle size (e.g., the mean particle size of the LNP in the reconstituted formulation) is between 80-120 nm. In some embodiments, the reconstituted LNP particle size (e.g., the mean particle size of the LNP in the reconstituted formulation) is between 80-115 nm.

### BRIEF DESCRIPTION OF DRAWINGS

FIG. 1A and FIG. 1B show Dry powder product (DPP) manufactured using mRNA-LNP formulations with xylitol and lactose, respectively, as excipients did not spray dry well. The liquid formulation adhered to the cyclone separator and no dry powder product was collected in the collection vessel.

FIG. 2A shows the aggregates observed in the reconstituted Dry Powder (DP) with 7% mannitol (w/v) as excipient under 400× magnification (right) as compared to water alone (left), as described in Example 2. FIG. 2B shows the appearances of reconstituted dry powder with 7% (left), 5% (middle), and 2.5% (right) mannitol (w/v) as excipient, as described in Example 2.

FIG. 3A shows no aggregates were observed in reconstituted DP with 5% sucrose (w/v) as excipient under microscope (right) as compared to water alone (left), as described in Example 2. FIG. 3B shows the appearances of reconstituted dry powder with 7% (left), 5% (middle), and 2.5% (right) sucrose (w/v) as excipient, as described in Example 2. FIG. 3C shows DPP manufactured using mRNA-LNP formulation with 2.5% sucrose (w/v) as excipient did not spray dry well and the liquid formulation adhered to the cyclone separator, as described in Example 2.

FIG. 4A shows the appearances of reconstituted dry powder with 7% (right), 5% (middle), and 2.5% (left) trehalose (w/v) as excipient, as described in Example 2. FIG. 4B shows the appearance of reconstituted dry powder with 1.25% trehalose (w/v) as excipient, as described in Example 2. FIG. 4C shows no aggregates were observed in reconstituted DP with 2.5% trehalose (w/v) as excipient under microscope (right) as compared to water alone (left), as described in Example 2. FIG. 4D shows DPP manufactured using mRNA-LNP formulation with 1.25% trehalose (w/v) as excipient did not spray dry well with most of the liquid formulation adhered to the cyclone separator and only little dry powder collected in the collection vessel (middle) as compared to mRNA-LNP formulation with 7% trehalose (w/v) as excipient (right), as described in Example 2.

FIG. 5 shows the appearances of reconstituted dry powder with trehalose added as part of the reconstituting solvent, as described in Example 2. Concentrations shown (7% and 5% (w/v)) are the concentrations of trehalose in the post reconstitution compositions.

FIG. 6A-FIG. 6D are graphs showing changes of particle size (FIG. 6A), polydispersity index (PDI; FIG. 6B), encapsulation efficiency (EE; FIG. 6C), and mRNA integrity (FIG. 6D) in post reconstitution compositions with 5% trehalose (w/v) during the course of 12 months at various temperatures (25° C., 4° C., or −20° C.).

FIG. 7A-FIG. 7C show mRNA integrity of post reconstitution compositions with 5% trehalose (w/v) as measured by capillary electrophoresis at 25° C., 4° C. and −20° C., respectively, after storage for 9 months. FIG. 7D-FIG. 7F show mRNA integrity of post reconstitution compositions with 5% trehalose (w/v) as measured by capillary electrophoresis at 25° C., 4° C. and −20° C., respectively, after storage for 6 months. FIG. 7G-FIG. 7I show mRNA integrity of post reconstitution compositions with 5% trehalose (w/v) as measured by capillary electrophoresis at 25° C., 4° C. and −20° C., respectively, after storage for 3 months. FIG. 7J shows the capillary electrophoresis plot of an mRNA standard as a control.

FIG. 8A is a graph showing post reconstitution of the trehalose dry powder (“Trehalose DP”) shows no change in mRNA integrity post spray drying as compared to a control mRNA (“Standard”), as described in Example 5. FIG. 8B is a graph showing OTC expression in mice after intravenous injection of a liquid control and the reconstituted trehalose dry powder.

FIG. 9 is a graph showing human erythropoietin (hEPO) expression in mice after intramuscular injection of a liquid control and the reconstituted trehalose dry powder.

FIG. 10A-FIG. 10C are graphs showing no significant change in particle size, PDI, encapsulation efficiency, and mRNA integrity for three different DPPs after storage at 2-8° C. over the course of 12 months, as described in Example 8. FIG. 10A also shows no significant change in the hEPO expression over the course of 12 months. FIG. 10A: DPP containing mRNA encoding hEPO (“cKK-E10-hEPO Dry Powder”); FIG. 10B: DPP containing mRNA encoding an influenza antigen (“cKK-E10-moono-Flu Dry Powder”);

FIG. 10C: DPP containing mRNA encoding an antigen from respiratory syncytial virus (RSV) antigen (“cKK-E10-RSV Dry Powder”)

FIG. 11A is a graph showing no significant change in particle size, PDI, encapsulation efficiency, and mRNA integrity for a DPP prepared with 4 different influenza mRNAs (QIV-Flu) after storage at 2-8° C. over the course of 10 months, as described in Example 8. FIG. 11B shows the capillary electrophoresis profiles of the QIV-Flu mRNA (“QIV mRNA”; top), the mRNA extracted from the reconstituted DPP at T0 (“Dry Powder TO”; middle), and the mRNA extracted from the reconstituted DPP after storage at 2-8° C. for 5.5 months (“Dry Powder 5.5 Months”; bottom).

### Definitions

In order for the present disclosure to be more readily understood, certain terms are first defined below. Additional definitions for the following terms and other terms are set forth throughout the specification. The publications and other reference materials referenced herein to describe the background of the disclosure and to provide additional detail regarding its practice are hereby incorporated by reference.

Approximately or about: As used herein, the term “approximately” or “about,” as applied to one or more values of interest, refers to a value that is similar to a stated reference value. In certain some embodiments, the term “approximately” or “about” refers to a range of values that fall within 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less, including all values and subranges therebetween, in either direction (greater than or less than) of the stated reference value unless otherwise stated or otherwise evident from the context (except where such number would exceed 100% of a possible value). In exemplary embodiments, about 5 refers to 4.5, 4.6, 4.7, 4.8, 4.9, 5.0, 5.1, 5.2, 5.3, 5.4, 5.5.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery). In some embodiments, delivery is oral delivery, intramuscular, intravenous, subcutaneous, sublingual, or buccal delivery.

Encapsulation: As used herein, the term “encapsulation,” or its grammatical equivalent, refers to the process of confining a nucleic acid molecule within a nanoparticle.

Encapsulation efficiency: As used herein, “encapsulation efficiency” or “EE” refers to the amount of a therapeutic and/or prophylactic, such as a ribonucleic acid molecule of the disclosure, that becomes part of a lipid nanoparticle (LNP), relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. Encapsulation efficiency can be determined by, for instance, the RiboGreen assay or any method known in the art. As used herein, “encapsulation” may refer to complete, substantial, or partial enclosure, confinement, surrounding, or encasement.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this disclosure, the terms “expression” and “production,” and their grammatical equivalents, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Mean particle size: As used herein, “mean particle size” in the context of lipid nanoparticle compositions refers to the mean diameter of a nanoparticle composition. “Mean particle size” in the context of dry powders in the dry powder formulations refers to the mean diameter of the dry powders.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Improved thermostability: As used herein, the term “improved thermostability” refers to the ability of a formulation (e.g., mRNA-nanoparticle or reconstituted mRNA-nanoparticle) to maintain its chemical structure and/or physical stability at elevated temperatures.

In some embodiments: As used herein, the term “in some embodiments,” or its grammatical equivalent, such as “in certain embodiments,” “in other embodiments,” “in some other embodiments,” or the like, refers to embodiments of all aspects of the disclosure, unless the context clearly indicates otherwise.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Liquid control: As used herein, the term “liquid control” or “liquid controls” refers to mRNA nanoparticle formulations directly formulated as liquid products, rather than dry powder formulations or reconstituted from dry powder formulations. Accordingly, as used herein, a liquid control formulation is distinct from a reconstituted formulation, which is adding at least one solvent to an mRNA encapsulated dry powder formulation.

Reconstitute: As used herein, the term “reconstitute” or “reconstituted” or related words refer to a process of adding a liquid diluent to a dry ingredient to make a specific concentration of liquid formulation.

Reconstituted formulation: As generally used herein, “reconstituted formulation” refers to the dissolution of a dry powder, freeze-dried protein, spray-dried protein or spray-dried nucleic acids (e.g., mRNA) or solvent-precipitated protein in a diluent. In some embodiments, “reconstituted formulation” refers to a formulation prepared by dissolving or dispersing the mRNA-nanoparticle in an aqueous solution for administration.

mRNA-LNP composition: As used herein, the term “mRNA-LNP composition,” or its grammatical equivalent, such as “mRNA-LNP formulation,” refers to a composition or formulation comprising one or more mRNA molecules encapsulated in LNPs.

Subject: As used herein, the term “subject” refers to a human or any non-human animal (e.g., mouse, rat, rabbit, dog, cat, cattle, swine, sheep, horse or primate) to which a provided composition (e.g., any described herein) may be administered, e.g., for experimental, diagnostic, prophylactic, cosmetic, and/or therapeutic purposes. A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder. Accordingly, a composition (e.g., any composition described herein) may be administered to a subject such as a patient (e.g., a human patient) in need thereof (e.g., in need of a therapeutic and/or prophylactic treatment as described herein).

Sugar: As used herein, the term “sugar” includes 1-10 monosaccharide units, e.g., monosaccharides, disaccharides, trisaccharides, and oligosaccharides comprising 4-10 monosaccharide units. Any sugar is useful in formulating the clostridial toxin pharmaceutical compositions disclosed herein, provided that a therapeutically effective amount of a clostridial toxin active ingredient, most preferably a botulinum toxin, is used using this sugar. It is assumed that it will be collected. In some embodiments, for example, in a lyophilized composition, the sugar may function as a lyoprotectant. In some other embodiments, the sugar may function as an isotonic agent, for example in lyophilized or liquid formulations. A monosaccharide is a cyclic type when a polyhydroxyaldehyde or polyhydroxyketone having 3 or more carbon atoms, such as aldose, dialdos, aldketose, ketose and diketose, and the parent monosaccharide has a (potential) carbonyl group, Deoxy sugars and amino sugars, and their derivatives. Trioses such as glyceraldehyde and dihydroxyacetone as monosaccharides; tetroses such as erythrose, erythrulose and threose; pentoses such as arabinose, lyxose, ribose, ribulose, xylose and xylulose; allose, altrose, fructose, fucose, galactose, glucose Hexoses, such as sucrose and mannoheptulose; octoses such as octulose and 2-keto-3-deoxy-mannooctonate; groose, idose, mannose, psicose, rhamnose, sorbose, tagatose, talose and trehalose; Nonoses such as sialose; and decourse. An oligosaccharide is a compound in which at least two types of monosaccharide units are linked by a glycosidic bond. Depending on the number of units, they are called disaccharides, trisaccharides, tetrasaccharides, pentasaccharides, hexasaccharides, heptasaccharides, decasaccharides, and the like. Oligosaccharides can be unbranched, branched, or cyclic. Common disaccharides include, but are not limited to, sucrose, lactose, maltose, trehalose, cellobiose, gentiobiose, cordierbiose, laminaribiose, mannobiose, melibiose, nigerose, rutinose, and xylobiose. Common trisaccharides include, but are not limited to, raffinose, acarbose, maltotriose, and meletitol. Other non-limiting examples of special purpose sugar excipients are described, for example, in Ansel (1999), Gennaro (2000), Hardman (2001), and Rowe (2003), each of which is incorporated by reference in its entirety.

Sugar alcohol: As used herein, the term “polyalcohol” is synonymous with “sugar alcohol”, “polyhydric alcohol”, and “polyol” and an alcohol group (CH 2 OH) instead of an aldehyde group (CHO). For example, mannose derived from mannose, xylitol derived from xylose, and lactitol derived from lactulose. Non-limiting examples of polyols include glycol, glycerol, arabitol, erythritol, xylitol, maltitol, sorbitol (glucitol), mannitol, inositol, lactitol, galactitol (iditol), isomalt. Other non-limiting examples of sugar excipients are described, for example, in Ansel (1999), Gennaro (2000), Hardman (2001), and Rowe (2003), each of which is incorporated by reference in its entirety

### DETAILED DESCRIPTION

The present application provides among other things, dry powder formulations of mRNA-LNPs that provide unexpectedly superior properties, including thermostable dry powder products (DPP) as well as reconstitutable dry powder products (reconstitutability and thermostability).

Dry powder product (DPP) and dry powder (DP) can be used interchangeably with the term “dry powder formulation” as used herein.

For example, dry powder formulations described herein can be suitable for use as a dry powder product without reconstitution as well as being suitable for use following reconstitution of the dry powder formulation as described herein. The ability to reconstitute dry powder formulations described herein is particularly beneficial for use of these formulations in various modes of delivery, including but not limited to parenteral (intramuscular, intravenous, subcutaneous, etc.) and mucosal (oral, sublingual, intranasal, etc.) delivery. The inventors of the present disclosure surprisingly found that the desired critical attributes for post-reconstitution product of dry powder formulations described herein were accomplished by using excipients described herein in the process of spray-drying as well as the weight ratio of the excipient to total lipid content in the DPP.

As disclosed herein, “reconstitutable dry powder formulation” and “dry powder formulation for reconstitution” can be used interchangeably.

Various exemplary aspects and embodiments of the disclosure are described in detail in the following sections. The use of sections is not meant to limit the disclosure. Each section can apply to any aspect of the disclosure. In this application, the use of “or” means “and/or” unless stated otherwise.

In some aspects, the disclosure features a dry powder formulation comprising a messenger RNA (mRNA) encapsulated in lipid nanoparticles (LNPs), where the lipid nanoparticles comprises one or more cationic lipids, one or more non-cationic lipids, and one or more PEG-modified lipids. In some embodiments, the formulation comprises a sugar or sugar alcohol, wherein the weight (w/w) ratio of the sugar or sugar alcohol and the total lipids in the lipid nanoparticles is at least about 5. In some embodiments, the dry powder formulation can be reconstituted for parenteral administration by reconstitution with water or a buffer. The reconstituted form of the dry powder formulation is sometimes called “reconstituted dry powder formulation.” In some embodiments, the dry powder formulation comprises dry powders having a particle size (e.g., mean particle size) of between 18 μm or between about 1 μm and about 8 μm.

In another aspect, the disclosure features a method of preparing a dry powder formulation as described herein. In some embodiments, the method comprises providing a first mixture comprising lipid nanoparticles encapsulating an mRNA (i.e., mRNA-LNP), adding an excipient selected from a sugar or sugar alcohol (e.g., trehalose) to the mRNA-LNP at a concentration of between 2 and 10% w/w, thereby obtaining a second mixture, and spray drying the second mixture. In some embodiments, a dry powder formulation is obtained that has a particle size of between 18 μm, e.g., the dry powders in the formulation have a mean particle size of between about 1 μm and about 8 μm. In some embodiments, the lipid nanoparticles used in the method of the disclosure comprises one or more cationic lipids, one or more non-cationic lipids, and one or more PEG-modified lipids. In some embodiments, the dry powder formulation can be reconstituted with water or buffer for parenteral administration. In some embodiments, the dry powder comprising the excipient is stable for at least 1 year (e.g., after storage at 2-8° C. for at least 1 year).

### Dry Powder Formulations Comprising A Sugar Or Sugar Alcohol

The present disclosure provides stable dry powder formulations containing mRNA-loaded lipid nanoparticles (mRNA-LNP) for therapeutic use. The dry powder formulations of the present disclosure are thermostable and/or reconstitutable dry powder formulations.

In some embodiments, the dry powder formulation of the present disclosure comprises excipients to enhance production of a thermostable and/or reconstitutable dry powder formulation. In some embodiments, the dry powder formulation of the present disclosure comprises excipients to enhance production of a reconstitutable dry powder formulation useful for parenteral administration. In some embodiments, the dry powder formulation described herein comprises a sugar and/or sugar alcohol as the excipient (e.g., trehalose or sucrose). In some embodiments, the dry powder formulation described herein comprises a sugar as the excipient. In some embodiments, the dry powder formulation described herein comprises a sugar alcohol as the excipient.

In some embodiment, the sugar is selected from glucose, fructose, mannose, galactose, mannitol, sorbitol, lactose, trehalose, sucrose, xylose, ribulose, maltose, tagatose, galactose, rhamnose, ribulose, threose, arabinose, xylose, lyxose, allose, altrose, idose, palatinose, reduced isomalto-oligosaccharides, reduced xylo-oligosaccharides, cellobiose, trehalose, raffinose, starch, dextran, maltodextrin, cyclodextrins, inulin, or any combination thereof. In some embodiments, the sugar alcohol is selected from erythritol, maltitol, mannitol, sorbitol, lactitol, xylitol, propylene glycol, glycerol (glycerin), threitol, galactitol, adonitol, dulcitol, pentaerythritol, or any combination thereof.

In some embodiments, the sugar or sugar alcohol is selected from trehalose, mannitol, lactose, xylitol, and sucrose, as well as any combinations thereof. In some embodiments, the sugar is trehalose. In some embodiments, the sugar or sugar alcohol is mannitol. In some embodiments, the sugar is lactose. In some embodiments, the sugar or sugar alcohol is xylitol. In some embodiments, the sugar or sugar alcohol is sucrose.

### Sugar Or Sugar Alcohol And Total Lipid

The excipient and the excipient to total lipid content ratio in the dry powder formulation can provide a reconstitutable dry powder with optimal properties. In some embodiments, the weight ratio (w/w) of the sugar or sugar alcohol (e.g., sucrose or trehalose) to the total lipids in the lipid nanoparticle provides for optimum stability of the reconstitutable dry powder formulation.

In some embodiments, the weight (w/w) ratio of the sugar or sugar alcohol to total lipids in the LNP is about 2-10, about 4-8, or about 4-6. In some embodiments, the weight (w/w) ratio of the sugar or sugar alcohol to total lipids in the LNP is about 2, about 3, about 4, about 5, about 6, about 7, about 8, about 9, or about 10. In some embodiments, the weight (w/w) ratio of the sugar or sugar alcohol to total lipids in the LNP is 2-10. In some embodiments, the w/w ratio of the sugar or sugar alcohol to total lipids in the LNP is 4-8. In some embodiments, the w/w ratio of the sugar or sugar alcohol to total lipids in the LNP is 4-6.

In some embodiments, the ratio of w/w ratio of the sugar or sugar alcohol to the total lipids in the lipid nanoparticle is at least about 2. In some embodiments, the ratio of w/w ratio of the sugar or sugar alcohol to the total lipids in the lipid nanoparticle is at least about 3. In some embodiments, the ratio of w/w ratio of the sugar or sugar alcohol to the total lipids in the lipid nanoparticle is at least about 4. In some embodiments, the ratio of w/w ratio of the sugar or sugar alcohol to the total lipids in the lipid nanoparticle is at least about 5. In some embodiments, the ratio of w/w ratio of the sugar or sugar alcohol to the total lipids in the lipid nanoparticle is at least about 6. In some embodiments, the ratio of w/w ratio of the sugar or sugar alcohol to the total lipids in the lipid nanoparticle is at least about 7. In some embodiments, the ratio of w/w ratio of the sugar or sugar alcohol to the total lipids in the lipid nanoparticle is at least about 8. In some embodiments, the ratio of w/w ratio of the sugar or sugar alcohol to the total lipids in the lipid nanoparticle is at least about 9. In some embodiments, the ratio of w/w ratio of the sugar or sugar alcohol to the total lipids in the lipid nanoparticle is at least about 10. In some embodiments, the ratio of w/w ratio of the sugar or sugar alcohol to the total lipids in the lipid nanoparticle is at least about 11. In some embodiments, the ratio of w/w ratio of the sugar or sugar alcohol to the total lipids in the lipid nanoparticle is at least about 12. In some embodiments, the ratio of w/w ratio of the sugar or sugar alcohol to the total lipids in the lipid nanoparticle is at least about 13. In some embodiments, the ratio of w/w ratio of the sugar or sugar alcohol to the total lipids in the lipid nanoparticle is at least about 14. In some embodiments, the ratio of w/w ratio of the sugar or sugar alcohol to the total lipids in the lipid nanoparticle is at least about 15. In some embodiments, the ratio of w/w ratio of the sugar or sugar alcohol to the total lipids in the lipid nanoparticle is at least about 16. In some embodiments, the ratio of w/w ratio of the sugar or sugar alcohol to the total lipids in the lipid nanoparticle is at least about 17. In some embodiments, the ratio of w/w ratio of the sugar or sugar alcohol to the total lipids in the lipid nanoparticle is at least about 18. In some embodiments, the ratio of w/w ratio of the sugar or sugar alcohol to the total lipids in the lipid nanoparticle is at least about 19. In some embodiments, the ratio of w/w ratio of the sugar or sugar alcohol to the total lipids in the lipid nanoparticle is at least about 20.

In some embodiments, the dry powder formulation comprises the w/w ratio of the excipient to the total lipids is at least 5. In some embodiments, the dry powder formulation comprises the w/w ratio of the excipient to the total lipids is at least 5.1. In some embodiments, the dry powder formulation comprises the w/w ratio of the excipient to the total lipids is at least 5.2. In some embodiments, the dry powder formulation comprises the w/w ratio of the excipient to the total lipids is at least 5.3. In some embodiments, the dry powder formulation comprises the w/w ratio of the excipient to the total lipids is at least 5.4.

In some embodiments, the dry powder formulation comprises the w/w ratio of the excipient to the total lipids is at least 5.5. In some embodiments, the dry powder formulation comprises the w/w ratio of the excipient to the total lipids is at least 5.6. In some embodiments, the dry powder formulation comprises the w/w ratio of the excipient to the total lipids is at least 5.7. In some embodiments, the dry powder formulation comprises the w/w ratio of the excipient to the total lipids is at least 5.8. In some embodiments, the dry powder formulation comprises the w/w ratio of the excipient to the total lipids is at least 5.9.

In some embodiments, the dry powder formulation comprises the w/w ratio of the excipient to the total lipids is at least 11.1. In some embodiments, the dry powder formulation comprises the w/w ratio of the excipient to the total lipids is at least 11.2. In some embodiments, the dry powder formulation comprises the w/w ratio of the excipient to the total lipids is at least 11.3. In some embodiments, the dry powder formulation comprises the w/w ratio of the excipient to the total lipids is at least 11.4. In some embodiments, the dry powder formulation comprises the w/w ratio of the excipient to the total lipids is at least 11.5. In some embodiments, the dry powder formulation comprises the w/w ratio of the excipient to the total lipids is at least 11.6. In some embodiments, the dry powder formulation comprises the w/w ratio of the excipient to the total lipids is at least 11.7. In some embodiments, the dry powder formulation comprises the w/w ratio of the excipient to the total lipids is at least 11.8. In some embodiments, the dry powder formulation comprises the w/w ratio of the excipient to the total lipids is at least 11.9.

In some embodiments, the dry powder formulation comprises the w/w ratio of the excipient to the total lipids is at least 15.0. In some embodiments, the dry powder formulation comprises the w/w ratio of the excipient to the total lipids is at least 15.1. In some embodiments, the dry powder formulation comprises the w/w ratio of the excipient to the total lipids is at least 15.2. In some embodiments, the dry powder formulation comprises the w/w ratio of the excipient to the total lipids is at least 15.3. In some embodiments, the dry powder formulation comprises the w/w ratio of the excipient to the total lipids is at least 15.4. In some embodiments, the dry powder formulation comprises the w/w ratio of the excipient to the total lipids is at least 15.5. In some embodiments, the dry powder formulation comprises the w/w ratio of the excipient to the total lipids is at least 15.6. In some embodiments, the dry powder formulation comprises the w/w ratio of the excipient to the total lipids is at least 15.7. In some embodiments, the dry powder formulation comprises the w/w ratio of the excipient to the total lipids is at least 15.8. In some embodiments, the dry powder formulation comprises the w/w ratio of the excipient to the total lipids is at least 15.9.

In some embodiments, the w/w ratio of the excipient to the total lipids in the LNP is at least 5. In some embodiments, the w/w ratio of the excipient to the total lipids in the LNP is at least 5.1. In some embodiments, the w/w ratio of the excipient to the total lipids in the LNP is at least 5.2. In some embodiments, the w/w ratio of the excipient to the total lipids in the LNP is at least 5.3. In some embodiments, the w/w ratio of the excipient to the total lipids in the LNP is at least 5.4. In some embodiments, the w/w ratio of the excipient to the total lipids in the LNP is at least 5.5. In some embodiments, the w/w ratio of the excipient to the total lipids in the LNP is at least 5.6. In some embodiments, the w/w ratio of the excipient to the total lipids in the LNP is at least 5.7. In some embodiments, the w/w ratio of the excipient to the total lipids in the LNP is at least 5.8. In some embodiments, the w/w ratio of the excipient to the total lipids in the LNP is at least 5.9.

In some embodiments, the w/w ratio of the excipient to the total lipids in the LNP is at least 11.1. In some embodiments, the w/w ratio of the excipient to the total lipids in the LNP is at least 11.2. In some embodiments, the w/w ratio of the excipient to the total lipids in the LNP is at least 11.3. In some embodiments, the w/w ratio of the excipient to the total lipids in the LNP is at least 11.4. In some embodiments, the w/w ratio of the excipient to the total lipids in the LNP is at least 11.5. In some embodiments, the w/w ratio of the excipient to the total lipids in the LNP is at least 11.6. In some embodiments, the w/w ratio of the excipient to the total lipids in the LNP is at least 11.7. In some embodiments, the w/w ratio of the excipient to the total lipids in the LNP is at least 11.8. In some embodiments, the w/w ratio of the excipient to the total lipids in the LNP is at least 11.9.

In some embodiments, the w/w ratio of the excipient to the total lipids in the LNP is at least 15.0. In some embodiments, the w/w ratio of the excipient to the total lipids in the LNP is at least 15.1. In some embodiments, the w/w ratio of the excipient to the total lipids in the LNP is at least 15.2. In some embodiments, the w/w ratio of the excipient to the total lipids in the LNP is at least 15.3. In some embodiments, the w/w ratio of the excipient to the total lipids in the LNP is at least 15.4. In some embodiments, the w/w ratio of the excipient to the total lipids in the LNP is at least 15.5. In some embodiments, the w/w ratio of the excipient to the total lipids in the LNP is at least 15.6. In some embodiments, the w/w ratio of the excipient to the total lipids in the LNP is at least 15.7. In some embodiments, the w/w ratio of the excipient to the total lipids in the LNP is at least 15.8. In some embodiments, the w/w ratio of the excipient to the total lipids in the LNP is at least 15.9.

In some embodiments, the sugar or sugar alcohol is selected from trehalose, mannitol, lactose, xylitol, and sucrose, as well as any combinations thereof.

### Exemplary Features of Dry Powder Formulations Described Herein

In some embodiments, a dry powder formulation (e.g., a reconstitutable dry powder formulation) comprising sugar or sugar alcohol disclosed herein has desirable properties including, but not limited to, better storage period, efficacy, thermostability, tissue absorption, and/or encapsulation efficacies. Exemplary, non-limiting beneficial features are described herein.

a. Storage

For example, a dry powder formulation as described herein can provide beneficial effects in the storage and/or storage stability, including but not limited to the exemplary embodiments described herein.

In some embodiments, a dry powder formulation as described herein can have an improved duration of storage (including, for example, at relatively elevated temperatures that do not require extreme cold storage). In some embodiments, a reconstitutable dry powder formulation as described herein is also characterized by their improved storage properties. For example, in certain embodiments, a reconstitutable dry powder formulation may be stored under refrigeration and remain stable (e.g., as demonstrated by minimal or no losses in their intended pharmaceutical or biological activity) for extended periods of time (e.g., stable for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 months, including any values and subranges therebetween, or longer upon storage at temperatures such as those described herein, including at about, for example 2-8° C. (e.g., 4° C.) or −20° C.). In other embodiments, a reconstitutable dry powder formulation described herein may be stored without refrigeration and remain stable for extended periods of time (e.g., stable for at least about 1, 2, 3, 4, 5, 7, 8, 9, 10, 11, 12, 13, 14, 15, 16, 17, 18, 19, 20, 21, 22, 23, 24, 25, 26, 27, 28, 29, 30, 31, 32, 33, 34, 35,, 36 months, including any values and subranges therebetween, or longer upon storage at ambient temperatures, including at about 25° C.).

In some embodiments, a reconstitutable dry powder formulation described herein provides comparable duration of storage as compared to an equivalent non-dry powder formulation. In some embodiments, the duration of storage does not appreciably change during storage of a dry powder formulation, including any exemplary, duration, and/or temperature described herein. In some embodiments, the duration of storage of a dry powder formulation described herein does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following storage, including according to any exemplary duration, and/or temperature as described herein.

In some embodiments, reconstitutable dry powder formulation described herein provides comparable duration of storage as compared to the dry powder formulation prior to reconstitution. In some embodiments, the duration of storage of a reconstituted dry powder formulation does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following reconstitution, including according to any method as described herein.

b. Stability

For example, a reconstitutable dry powder formulation as described herein can provide beneficial effects in the stability of the formulation, including but not limited to the exemplary embodiments described herein.

In some embodiments, the reconstitutable dry powder formulation comprising sugar or sugar alcohol as described herein is stable for at least 1 week. In some embodiments, the reconstitutable dry powder formulation is stable for at least 2 weeks. In some embodiments, the reconstitutable dry powder formulation is stable for at least 3 weeks. In some embodiments, the reconstitutable dry powder formulation is stable for at least 4 weeks. In some embodiments, the reconstitutable dry powder formulation is stable for at least 5 weeks. In some embodiments, the reconstitutable dry powder formulation is stable for at least 1 month. In some embodiments, the reconstitutable dry powder formulation is stable for at least 2 months. In some embodiments, the reconstitutable dry powder formulation is stable for at least 3 months. In some embodiments, the reconstitutable dry powder formulation is stable for at least 4 months. In some embodiments, the reconstitutable dry powder formulation is stable for at least 5 months. In some embodiments, the reconstitutable dry powder formulation is stable for at least 6 months. In some embodiments, the reconstitutable dry powder formulation is stable for at least 7 months. In some embodiments, the reconstitutable dry powder formulation is stable for at least 8 months. In some embodiments, the reconstitutable dry powder formulation is stable for at least 9 months. In some embodiments, the reconstitutable dry powder formulation is stable for at least 10 months. In some embodiments, the reconstitutable dry powder formulation is stable for at least 11 months. In some embodiments, the reconstitutable dry powder formulation is stable for at least 1 year. In some embodiments, the reconstitutable dry powder formulation is stable for about 1 year or more.

In some embodiments, the stability of the reconstitutable dry powder formulation comprising sugar or sugar alcohol as described herein is increased by at least about 1 week compared to liquid controls. In some embodiments, the stability of the reconstitutable dry powder formulation is increased by at least 2 weeks compared to liquid controls. In some embodiments, the stability of the reconstitutable dry powder formulation is increased by at least 3 weeks compared to liquid controls. In some embodiments, the stability of the reconstitutable dry powder formulation is increased by at least 4 weeks compared to liquid controls. In some embodiments, the stability of the reconstitutable dry powder formulation is increased by at least about 5 weeks compared to liquid controls. In some embodiments, the stability of the reconstitutable dry powder is increased by at least 1 month compared to liquid controls. In some embodiments, the stability of the reconstitutable dry powder is increased by at least 2 months compared to liquid controls. In some embodiments, the stability of the reconstitutable dry powder formulation is increased by at least 3 months compared to liquid controls. In some embodiments, the stability of the reconstitutable dry powder is increased by at least 4 months compared to liquid controls. In some embodiments, the stability of the reconstitutable dry powder is increased by at least 5 months compared to liquid controls. In some embodiments, the stability of the reconstitutable dry powder formulation is increased by at least 6 months compared to liquid controls. In some embodiments, the stability of the reconstitutable dry powder is increased by at least 7 months compared to liquid controls. In some embodiments, the stability of the reconstitutable dry powder is increased by at least 8 months compared to liquid controls. In the stability of the reconstitutable dry powder formulation is increased by at least 9 months compared to liquid controls. In some embodiments, the stability of the reconstitutable dry powder is increased by at least 10 months compared to liquid controls. In some embodiments, the stability of the reconstitutable dry powder is increased by at least 11 months compared to liquid controls. In some embodiments, the stability of the reconstitutable dry powder formulation is increased by at least 1 year compared to liquid controls. In some embodiments, the stability of the reconstitutable dry powder formulation is increased by 1 year or more compared to liquid controls.

In some embodiments, the reconstitutable dry powder formulation comprising sugar or sugar alcohol as described herein comprises improved thermostability when compared to liquid controls. In some embodiments, the reconstitutable dry powder formulation is stable at 4° C. for at least 1 week. In some embodiments, reconstitutable the dry powder formulation is stable at 4° C. for at least 2 weeks. In some embodiments, the reconstitutable dry powder formulation is stable at 4° C. for at least 3 weeks. In some embodiments, the reconstitutable dry powder formulation is stable at 4° C. for at least 4 weeks. In some embodiments, the reconstitutable dry powder formulation is stable at 4° C. for at least about 5 weeks. In some embodiments, the reconstitutable dry powder formulation is stable at 4° C. for 1 month. In some embodiments, the reconstitutable dry powder formulation is stable at 4° C. for at least 2 months. In some embodiments, the reconstitutable dry powder formulation is stable at 4° C. for at least 3 months. In some embodiments, the reconstitutable dry powder formulation is stable at 4° C. for at least 4 months. In some embodiments, the reconstitutable dry powder formulation is stable at 4° C. for at least 5 months. In some embodiments, the reconstitutable dry powder formulation is stable at 4° C. for at least 6 months. In some embodiments, the reconstitutable dry powder formulation is stable at 4° C. for at least 7 months. In some embodiments, the reconstitutable dry powder formulation is stable at 4° C. for at least 8 months. In some embodiments, the reconstitutable dry powder formulation is stable at 4° C. for at least 9 months. In some embodiments, the reconstitutable dry powder formulation is stable at 4° C. for at least 10 months. In some embodiments, the reconstitutable dry powder formulation is stable at 4° C. for at least 11 months. In some embodiments, the reconstitutable dry powder formulation is stable at 4° C. for at least 1 year. In some embodiments, the reconstitutable dry powder formulation is stable at 4° C. for more than 1 year.

In some embodiments, the reconstitutable dry powder formulation comprising sugar or sugar alcohol as described herein is stable at 25° C. for at least 1 week. In some embodiments, the reconstitutable dry powder formulation is stable at 25° C. for at least 2 weeks. In some embodiments, the reconstitutable dry powder formulation is stable at 25° C. for at least 3 weeks. In some embodiments, the reconstitutable dry powder formulation is stable at 25° C. for at least 4 weeks. In some embodiments, the reconstitutable dry powder formulation is stable at 25° C. for at least about 5 weeks. In some embodiments, the reconstitutable dry powder formulation is stable at 25° C. for 1 month. In some embodiments, the reconstitutable dry powder formulation is stable at 25° C. for at least 2 months. In some embodiments, the reconstitutable dry powder formulation is stable at 25° C. for at least 3 months. In some embodiments, the reconstitutable dry powder formulation is stable at 25° C. for at least 4 months. In some embodiments, the reconstitutable dry powder formulation is stable at 25° C. for at least 5 months. In some embodiments, the reconstitutable dry powder formulation is stable at 25° C. for at least 6 months. In some embodiments, the reconstitutable dry powder formulation is stable at 25° C. for at least 7 months. In some embodiments, the reconstitutable dry powder formulation is stable at 25° C. for at least 8 months. In some embodiments, the reconstitutable dry powder formulation is stable at 25° C. for at least 9 months. In some embodiments, the reconstitutable dry powder formulation is stable at 25° C. for at least 10 months. In some embodiments, the reconstitutable dry powder formulation is stable at 25° C. for at least 11 months. In some embodiments, the reconstitutable dry powder formulation is stable at 25° C. for at least 1 year. In some embodiments, the reconstitutable dry powder formulation is stable at 25° C. for more than 1 year.

In some embodiments, the reconstitutable dry powder formulation comprising sugar or sugar alcohol as described herein is stable at −20° C. for at least 1 week. In some embodiments, the reconstitutable dry powder formulation is stable at −20° C. for at least 2 weeks. In some embodiments, the reconstitutable dry powder formulation is stable at −20° C. for at least 3 weeks. In some embodiments, the reconstitutable dry powder formulation is stable at −20° C. for at least 4 weeks. In some embodiments, the reconstitutable dry powder formulation is stable at −20° C. for at least 5 weeks. In some embodiments, the reconstitutable dry powder formulation is stable at −20° C. for 1 month. In some embodiments, the reconstitutable dry powder formulation is stable at −20° C. for at least 2 months. In some embodiments, the reconstitutable dry powder formulation is stable at −20° C. for at least 3 months. In some embodiments, the reconstitutable dry powder formulation is stable at −20° C. for at least 4 months. In some embodiments, the reconstitutable dry powder formulation is stable at −20° C. for at least 5 months. In some embodiments, the reconstitutable dry powder formulation is stable at −20° C. for at least 6 months. In some embodiments, the reconstitutable dry powder formulation is stable at −20° C. for at least 7 months. In some embodiments, the reconstitutable dry powder formulation is stable at −20° C. for at least 8 months. In some embodiments, the reconstitutable dry powder formulation is stable at −20° C. for at least 9 months. In some embodiments, the reconstitutable dry powder formulation is stable at −20° C. for at least 10 months. In some embodiments, the reconstitutable dry powder formulation is stable at −20° C. for at least 11 months. In some embodiments, the reconstitutable dry powder formulation is stable at −20° C. for at least 1 year. In some embodiments, the reconstitutable dry powder formulation is stable at −20° C. for more than 1 year.

In certain some embodiments, a reconstitutable dry powder formulation described herein demonstrates a negligible reduction in pharmacological or biological activity (e.g., less than about a 1%, 2%, 2.5%, 4%, 5%, 7.5%, 10%, 12.5%, 15%, 18.5%, 20%, 25%, 30%, 35%, 40% or 50%, including all values and ranges therebetween, reduction in the biological or pharmacological activity of an encapsulated polynucleotide) (e.g., as compared to prior to dry powder formulation or a control formulation without spray-drying or the dry powder formulation prior to storage).

In some embodiments, a reconstitutable dry powder formulation described herein provides comparable stability as compared to an equivalent non-dry powder formulation. In some embodiments, the stability does not appreciably change during storage of the dry powder formulation, including any exemplary, storage, duration, and/or temperature described herein. In some embodiments, the stability of the dry powder formulation does not change by more than about 1%, 2%, 2.5%, 4%, 5%, 7.5%, 10%, 12.5%, 15%, 18.5%, 20%, 25%, 30%, 35%, 40% or 50%, including all values and ranges therebetween (e.g., no more than about 30%, 25%, 20%, 15%, 10%, or 5%), following storage, including according to any exemplary storage, duration, and/or temperature as described herein.

In some embodiments, a reconstitutable dry powder formulation described herein provides comparable stability as compared to the dry powder formulation prior to reconstitution. In some embodiments, the stability of a reconstitutable dry powder formulation does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following reconstitution, including according to any method as described herein.

c. mRNA Integrity

For example, a dry powder formulation as described herein can provide beneficial effects in maintaining the integrity of the mRNA encapsulated within the LNP, including but not limited to the exemplary embodiments described herein.

In some embodiments, the mRNA maintains an integrity of 60% or greater after storage for 1, 2, 3, 4, 5, 6, 9, 12, 18, 24, 36 months or longer, including all values and ranges therebetween. In some embodiments, the mRNA maintains an integrity of 61% or greater after storage for 1, 2, 3, 4, 5, 6, 9, 12, 18, 24, 36 months or longer, including all values and ranges therebetween. In some embodiments, the mRNA maintains an integrity of 62% or greater after storage for 1, 2, 3, 4, 5, 6, 9, 12, 18, 24, 36 months or longer, including all values and ranges therebetween. In some embodiments, the mRNA maintains an integrity of 63% or greater after storage for 1, 2, 3, 4, 5, 6, 9, 12, 18, 24, 36 months or longer, including all values and ranges therebetween. In some embodiments, the mRNA maintains an integrity of 64% or greater after storage for 1, 2, 3, 4, 5, 6, 9, 12, 18, 24, 36 months or longer, including all values and ranges therebetween. In some embodiments, the mRNA maintains an integrity of 65% or greater after storage for 1, 2, 3, 4, 5, 6, 9, 12, 18, 24, 36 months or longer, including all values and ranges therebetween. In some embodiments, the mRNA maintains an integrity of 66% or greater after storage for 1, 2, 3, 4, 5, 6, 9, 12, 18, 24, 36 months or longer, including all values and ranges therebetween. In some embodiments, the mRNA maintains an integrity of 67% or greater after storage for 1, 2, 3, 4, 5, 6, 9, 12, 18, 24, 36 months or longer, including all values and ranges therebetween. In some embodiments, the mRNA maintains an integrity of 68% or greater after storage for 1, 2, 3, 4, 5, 6, 9, 12, 18, 24, 36 months or longer, including all values and ranges therebetween. In some embodiments, the mRNA maintains an integrity of 69% or greater after storage for 1, 2, 3, 4, 5, 6, 9, 12, 18, 24, 36 months or longer, including all values and ranges therebetween. In some embodiments, the mRNA maintains an integrity of 70% or greater after storage for 1, 2, 3, 4, 5, 6, 9, 12, 18, 24, 36 months or longer, including all values and ranges therebetween. In some embodiments, the mRNA maintains an integrity of 71% or greater after storage for 1, 2, 3, 4, 5, 6, 9, 12, 18, 24, 36 months or longer, including all values and ranges therebetween. In some embodiments, the mRNA maintains an integrity of 72% or greater after storage for 1, 2, 3, 4, 5, 6, 9, 12, 18, 24, 36 months or longer, including all values and ranges therebetween. In some embodiments, the mRNA maintains an integrity of 73% or greater after storage for 1, 2, 3, 4, 5, 6, 9, 12, 18, 24, 36 months or longer, including all values and ranges therebetween. In some embodiments, the mRNA maintains an integrity of 74% or greater after storage for 1, 2, 3, 4, 5, 6, 9, 12, 18, 24, 36 months or longer, including all values and ranges therebetween. In some embodiments, the mRNA maintains an integrity of 75% or greater after storage for 1, 2, 3, 4, 5, 6, 9, 12, 18, 24, 36 months or longer, including all values and ranges therebetween. In some embodiments, the mRNA maintains an integrity of 76% or greater after storage for 1, 2, 3, 4, 5, 6, 9, 12, 18, 24, 36 months or longer, including all values and ranges therebetween. In some embodiments, the mRNA maintains an integrity of 77% or greater after storage for 1, 2, 3, 4, 5, 6, 9, 12, 18, 24, 36 months or longer, including all values and ranges therebetween. In some embodiments, the mRNA maintains an integrity of 78% or greater after storage for 1, 2, 3, 4, 5, 6, 9, 12, 18, 24, 36 months or longer, including all values and ranges therebetween. In some embodiments, the mRNA maintains an integrity of 79% or greater after storage for 1, 2, 3, 4, 5, 6, 9, 12, 18, 24, 36 months or longer, including all values and ranges therebetween. In some embodiments, the mRNA maintains an integrity of 80% or greater after storage for 1, 2, 3, 4, 5, 6, 9, 12, 18, 24, 36 months or longer, including all values and ranges therebetween. In some embodiments, the mRNA maintains an integrity of 81% or greater after storage for 1, 2, 3, 4, 5, 6, 9, 12, 18, 24, 36 months or longer, including all values and ranges therebetween. In some embodiments, the mRNA maintains an integrity of 82% or greater after storage for 1, 2, 3, 4, 5, 6, 9, 12, 18, 24, 36 months or longer, including all values and ranges therebetween. In some embodiments, the mRNA maintains an integrity of 83% or greater after storage for 1, 2, 3, 4, 5, 6, 9, 12, 18, 24, 36 months or longer, including all values and ranges therebetween. In some embodiments, the mRNA maintains an integrity of 84% or greater after storage for 1, 2, 3, 4, 5, 6, 9, 12, 18, 24, 36 months or longer, including all values and ranges therebetween. In some embodiments, the mRNA maintains an integrity of 85% or greater after storage for 1, 2, 3, 4, 5, 6, 9, 12, 18, 24, 36 months or longer, including all values and ranges therebetween. In some embodiments, the mRNA maintains an integrity of 86% or greater after storage for 1, 2, 3, 4, 5, 6, 9, 12, 18, 24, 36 months or longer, including all values and ranges therebetween. In some embodiments, the mRNA maintains an integrity of 87% or greater after storage for 1, 2, 3, 4, 5, 6, 9, 12, 18, 24, 36 months or longer, including all values and ranges therebetween. In some embodiments, the mRNA maintains an integrity of 88% or greater after storage for 1, 2, 3, 4, 5, 6, 9, 12, 18, 24, 36 months or longer, including all values and ranges therebetween. In some embodiments, the mRNA maintains an integrity of 89% or greater after storage for 1, 2, 3, 4, 5, 6, 9, 12, 18, 24, 36 months or longer, including all values and ranges therebetween. In some embodiments, the mRNA maintains an integrity of 90% or greater for 1, 2, 3, 4, 5, 6, 9, 12, 18, 24, 36 months or longer, including all values and ranges therebetween.

In some embodiments, the mRNA maintains an integrity of 60% or greater after storage at 4° C. for at least six months. In some embodiments, the mRNA maintains an integrity of 61% or greater after storage at 4° C. for at least six months. In some embodiments, the mRNA maintains an integrity of 62% or greater after storage at 4° C. for at least six months. In some embodiments, the mRNA maintains an integrity of 63% or greater after storage at 4° C. for at least six months. In some embodiments, the mRNA maintains an integrity of 64% or greater after storage at 4° C. for at least six months. In some embodiments, the mRNA maintains an integrity of 65% or greater after storage at 4° C. for at least six months. In some embodiments, the mRNA maintains an integrity of 66% or greater after storage at 4° C. for at least six months. In some embodiments, the mRNA maintains an integrity of 67% or greater after storage at 4° C. for at least six months. In some embodiments, the mRNA maintains an integrity of 68% or greater after storage at 4° C. for at least six months. In some embodiments, the mRNA maintains an integrity of 69% or greater after storage at 4° C. for at least six months. In some embodiments, the mRNA maintains an integrity of 70% or greater after storage at 4° C. for at least six months. In some embodiments, the mRNA maintains an integrity of 71% or greater after storage at 4° C. for at least six months. In some embodiments, the mRNA maintains an integrity of 72% or greater after storage at 4° C. for at least six months. In some embodiments, the mRNA maintains an integrity of 73% or greater after storage at 4° C. for at least six months. In some embodiments, the mRNA maintains an integrity of 74% or greater after storage at 4° C. for at least six months. In some embodiments, the mRNA maintains an integrity of 75% or greater after storage at 4° C. for at least six months. In some embodiments, the mRNA maintains an integrity of 76% or greater after storage at 4° C. for at least six months. In some embodiments, the mRNA maintains an integrity of 77% or greater after storage at 4° C. for at least six months. In some embodiments, the mRNA maintains an integrity of 78% or greater after storage at 4° C. for at least six months. In some embodiments, the mRNA maintains an integrity of 79% or greater after storage at 4° C. for at least six months. In some embodiments, the mRNA maintains an integrity of 80% or greater after storage at 4° C. for at least six months. In some embodiments, the mRNA maintains an integrity of 81% or greater after storage at 4° C. for at least six months. In some embodiments, the mRNA maintains an integrity of 82% or greater after storage at 4° C. for at least six months. In some embodiments, the mRNA maintains an integrity of 83% or greater after storage at 4° C. for at least six months. In some embodiments, the mRNA maintains an integrity of 84% or greater after storage at 4° C. for at least six months. In some embodiments, the mRNA maintains an integrity of 85% or greater after storage at 4° C. for at least six months. In some embodiments, the mRNA maintains an integrity of 86% or greater after storage at 4° C. for at least six months. In some embodiments, the mRNA maintains an integrity of 87% or greater after storage at 4° C. for at least six months. In some embodiments, the mRNA maintains an integrity of 88% or greater after storage at 4° C. for at least six months. In some embodiments, the mRNA maintains an integrity of 89% or greater after storage at 4° C. for at least six months. In some embodiments, the mRNA maintains an integrity of 90% or greater after storage at 4° C. for at least six months.

In some embodiments, the mRNA maintains an integrity of 60% or greater after storage at 25° C. for at least six months. In some embodiments, the mRNA maintains an integrity of 61% or greater after storage at 25° C. for at least six months. In some embodiments, the mRNA maintains an integrity of 62% or greater after storage at 25° C. for at least six months. In some embodiments, the mRNA maintains an integrity of 63% or greater after storage at 25° C. for at least six months. In some embodiments, the mRNA maintains an integrity of 64% or greater after storage at 25° C. for at least six months. In some embodiments, the mRNA maintains an integrity of 65% or greater after storage at 25° C. for at least six months. In some embodiments, the mRNA maintains an integrity of 66% or greater after storage at 25° C. for at least six months. In some embodiments, the mRNA maintains an integrity of 67% or greater after storage at 25° C. for at least six months. In some embodiments, the mRNA maintains an integrity of 68% or greater after storage at 25° C. for at least six months. In some embodiments, the mRNA maintains an integrity of 69% or greater after storage at 25° C. for at least six months. In some embodiments, the mRNA maintains an integrity of 70% or greater after storage at 25° C. for at least six months. In some embodiments, the mRNA maintains an integrity of 71% or greater after storage at 25° C. for at least six months. In some embodiments, the mRNA maintains an integrity of 72% or greater after storage at 25° C. for at least six months. In some embodiments, the mRNA maintains an integrity of 73% or greater after storage at 25° C. for at least six months. In some embodiments, the mRNA maintains an integrity of 74% or greater after storage at 25° C. for at least six months. In some embodiments, the mRNA maintains an integrity of 75% or greater after storage at 25° C. for at least six months. In some embodiments, the mRNA maintains an integrity of 76% or greater after storage at 25° C. for at least six months. In some embodiments, the mRNA maintains an integrity of 77% or greater after storage at 25° C. for at least six months. In some embodiments, the mRNA maintains an integrity of 78% or greater after storage at 25° C. for at least six months. In some embodiments, the mRNA maintains an integrity of 79% or greater after storage at 25° C. for at least six months. In some embodiments, the mRNA maintains an integrity of 80% or greater after storage at 25° C. for at least six months. In some embodiments, the mRNA maintains an integrity of 81% or greater after storage at 25° C. for at least six months. In some embodiments, the mRNA maintains an integrity of 82% or greater after storage at 25° C. for at least six months. In some embodiments, the mRNA maintains an integrity of 83% or greater after storage at 25° C. for at least six months. In some embodiments, the mRNA maintains an integrity of 84% or greater after storage at 25° C. for at least six months. In some embodiments, the mRNA maintains an integrity of 85% or greater after storage at 25° C. for at least six months. In some embodiments, the mRNA maintains an integrity of 86% or greater after storage at 25° C. for at least six months. In some embodiments, the mRNA maintains an integrity of 87% or greater after storage at 25° C. for at least six months. In some embodiments, the mRNA maintains an integrity of 88% or greater after storage at 25° C. for at least six months. In some embodiments, the mRNA maintains an integrity of 89% or greater after storage at 25° C. for at least six months. In some embodiments, the mRNA maintains an integrity of 90% or greater after storage at 25° C. for at least six months.

In some embodiments, the mRNA maintains an integrity of 60% or greater after storage at −20° C. for at least six months. In some embodiments, the mRNA maintains an integrity of 61% or greater after storage at −20° C. for at least six months. In some embodiments, the mRNA maintains an integrity of 62% or greater after storage at −20° C. for at least six months. In some embodiments, the mRNA maintains an integrity of 63% or greater after storage at −20° C. for at least six months. In some embodiments, the mRNA maintains an integrity of 64% or greater after storage at −20° C. for at least six months. In some embodiments, the mRNA maintains an integrity of 65% or greater after storage at −20° C. for at least six months. In some embodiments, the mRNA maintains an integrity of 66% or greater after storage at −20° C. for at least six months. In some embodiments, the mRNA maintains an integrity of 67% or greater after storage at −20° C. for at least six months. In some embodiments, the mRNA maintains an integrity of 68% or greater after storage at −20° C. for at least six months. In some embodiments, the mRNA maintains an integrity of 69% or greater after storage at −20° C. for at least six months. In some embodiments, the mRNA maintains an integrity of 70% or greater after storage at −20° C. for at least six months. In some embodiments, the mRNA maintains an integrity of 71% or greater after storage at −20° C. for at least six months. In some embodiments, the mRNA maintains an integrity of 72% or greater after storage at −20° C. for at least six months. In some embodiments, the mRNA maintains an integrity of 73% or greater after storage at −20° C. for at least six months. In some embodiments, the mRNA maintains an integrity of 74% or greater after storage at −20° C. for at least six months. In some embodiments, the mRNA maintains an integrity of 75% or greater after storage at −20° C. for at least six months. In some embodiments, the mRNA maintains an integrity of 76% or greater after storage at −20° C. for at least six months. In some embodiments, the mRNA maintains an integrity of 77% or greater after storage at −20° C. for at least six months. In some embodiments, the mRNA maintains an integrity of 78% or greater after storage at −20° C. for at least six months. In some embodiments, the mRNA maintains an integrity of 79% or greater after storage at −20° C. for at least six months. In some embodiments, the mRNA maintains an integrity of 80% or greater after storage at-20° C. for at least six months. In some embodiments, the mRNA maintains an integrity of 81% or greater after storage at −20° C. for at least six months. In some embodiments, the mRNA maintains an integrity of 82% or greater after storage at −20° C. for at least six months. In some embodiments, the mRNA maintains an integrity of 83% or greater after storage at-20° C. for at least six months. In some embodiments, the mRNA maintains an integrity of 84% or greater after storage at −20° C. for at least six months. In some embodiments, the mRNA maintains an integrity of 85% or greater after storage at −20° C. for at least six months. In some embodiments, the mRNA maintains an integrity of 86% or greater after storage at-20° C. for at least six months. In some embodiments, the mRNA maintains an integrity of 87% or greater after storage at −20° C. for at least six months. In some embodiments, the mRNA maintains an integrity of 88% or greater after storage at −20° C. for at least six months. In some embodiments, the mRNA maintains an integrity of 89% or greater after storage at-20° C. for at least six months. In some embodiments, the mRNA maintains an integrity of 90% or greater after storage at −20° C. for at least six months.

In some embodiments, the mRNA maintains an integrity of about 60% or greater, such as 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, including any values and subranges therebetween, after storage at 2-8° C. (e.g., about 4° C.) for at least about a year.

In some embodiments, the mRNA maintains an integrity of about 70% or greater after storage at 2-8° C. for at least about a year. In some embodiments, the mRNA maintains an integrity of about 75% or greater after storage at 2-8° C. for at least about a year. In some embodiments, the mRNA maintains an integrity of about 80% or greater after storage at 2-8° C. for at least about a year. In some embodiments, the mRNA maintains an integrity of about 85% or greater after storage at 2-8° C. for at least about a year. In some embodiments, the mRNA maintains an integrity of about 90% or greater after storage at 2-8° C. for at least about a year. In some embodiments, the mRNA maintains an integrity of about 95% or greater after storage at 2-8° C. for at least about a year.

In some embodiments, the mRNA maintains an integrity of about 70% or greater after storage at 4° C. for at least about a year. In some embodiments, the mRNA maintains an integrity of about 75% or greater after storage at 4° C. for at least about a year. In some embodiments, the mRNA maintains an integrity of about 80% or greater after storage at 4° C. for at least about a year. In some embodiments, the mRNA maintains an integrity of about 85% or greater after storage at 4° C. for at least about a year. In some embodiments, the mRNA maintains an integrity of about 90% or greater after storage at 4° C. for at least about a year. In some embodiments, the mRNA maintains an integrity of about 95% or greater after storage at 4° C. for at least about a year.

In some embodiments, a reconstitutable dry powder formulation described herein provides comparable mRNA integrity as compared to an equivalent non-dry powder formulation. In some embodiments, the mRNA integrity does not appreciably change during storage of the reconstitutable dry powder formulation, including any exemplary, storage, duration, and/or temperature described herein. In some embodiments, the mRNA integrity of the reconstitutable dry powder formulation does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following storage, including according to any exemplary storage, duration, and/or temperature as described herein.

In some embodiments, a reconstituted dry powder formulation described herein provides comparable mRNA integrity as compared to the dry powder formulation prior to reconstitution. In some embodiments, the mRNA integrity of a reconstituted dry powder formulation does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following reconstitution, including according to any method as described herein.

d. Particle Size

For example, a reconstitutable dry powder formulation as described herein can provide beneficial effects in the particle size of the LNPs of the formulation, e.g., in maintaining the particle size (e.g., mean particle size) of the LNPs, including but not limited to the exemplary embodiments described herein.

In some embodiments, the reconstitutable dry powder formulation described herein comprises dry powders having a particle size (e.g., mean particle size) of between 0.5-10 μm. In some embodiments, the particle size (e.g., mean particle size) of the dry powders in the formulation is between 1-5 μm. In some embodiments, the particle size (e.g., mean particle size) of the dry powders in the formulation is between 1-4 μm. In some embodiments, the particle size (e.g., mean particle size) of the dry powders in the formulation is between 1-3 μm. In some embodiments, the particle size (e.g., mean particle size) of the dry powders in the formulation is between 1-2 μm. In some embodiments, the particle size (e.g., mean particle size) of the dry powders in the formulation is about 1 μm. In some embodiments, the particle size (e.g., mean particle size) of the dry powders in the formulation is about 2 μm. In some embodiments, the particle size (e.g., mean particle size) of the dry powders in the formulation is about 3 μm. In some embodiments, the particle size (e.g., mean particle size) of the dry powders in the formulation is about 4 μm. In some embodiments, the particle size (e.g., mean particle size) of the dry powders in the formulation is about 5 μm. In some embodiments, the particle size (e.g., mean particle size) of the dry powders in the formulation is about 6 μm. In some embodiments, the particle size (e.g., mean particle size) of the dry powders in the formulation is about 7 μm. In some embodiments, the particle size (e.g., mean particle size) of the dry powders in the formulation is about 8 μm. In some embodiments, the particle size (e.g., mean particle size) of the dry powders in the formulation is about 9 μm. In some embodiments, the particle size (e.g., mean particle size) of the dry powders in the formulation is about 10 μm.

In some embodiments, the particle size (e.g., mean particle size) of the dry powders in the reconstitutable dry powder formulation disclosed herein is desirable for delivery into various tissues. For example, dry powder formulations comprising particle size (e.g., mean particle size) of about 5 microns or less are especially useful for pulmonary delivery. In additional examples, dry powder formulations comprising trehalose have been found to have a particle size of 5 microns or less and may be suited to pulmonary delivery.

In some embodiments, a reconstitutable dry powder formulation described herein provides comparable particle size (e.g., mean particle size) as compared to an equivalent non-dry powder formulation. In some embodiments, the particle size (e.g., mean particle size) does not appreciably change during storage of a dry powder formulation, including any exemplary storage, duration, and/or temperature described herein. In some embodiments, the particle size (e.g., mean particle size) of a reconstitutable dry powder formulation does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following storage, including according to any exemplary storage, duration, and/or temperature as described herein.

In some embodiments, reconstitutable dry powder formulation described herein provides comparable particle size (e.g., mean particle size) as compared to the dry powder formulation prior to reconstitution. In some embodiments, the particle size (e.g., mean particle size) of a reconstitutable dry powder formulation does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following reconstitution, including according to any method as described herein.

e. Delivery Routes

Reconstitutable dry powder formulations described herein can be useful in various delivery routes. Exemplary embodiments are described herein.

In some embodiments, reconstitutable dry powder formulations comprising sugar or sugar alcohol as described herein enable administration of the mRNA-nanoparticle as a liquid formulation at a desirable dosage. In some embodiments, the reconstituable dry powder formulation with dry powders having 6 microns or less, 5 microns or less, 4 microns or less, 3 microns or less, 2 microns or less, 1 micron or less, including all values and ranges therebetween, may be suitable for pulmonary delivery. The pharmaceutical compositions comprising the dry powder formulations for reconstitution as described herein may be administered to a subject in need thereof subconjunctival, sublingual by any one of several routes that effectively delivers an effective amount of the compound. Non-limiting examples of suitable administrative routes include topical, oral, nasal, intrathecal, enteral, buccal, sublingual, transdermal, rectal, vaginal, intraocular, and parenteral administration, including subcutaneous, intravenous, intramuscular, intrasternal, intracavemous, intrameatal, and intraurethral injection and/or infusion.

In some embodiments, the reconstitutable dry powder formulation is suitable for mucosal delivery. In some embodiments, the reconstitutable dry powder formulation is suitable for oral delivery. In some embodiments, the reconstitutable dry powder formulation is suitable for sublingual delivery. In some embodiments, the reconstitutable dry powder formulation is suitable for or intranasal delivery. In some embodiments, the reconstitutable dry powder formulation is suitable for buccal delivery.

f. Improved Delivery Efficacy

A reconstituable dry powder formulation as described herein can provide beneficial effects in the delivery of the formulation to the target tissues, including but not limited to the exemplary embodiments described herein.

In some embodiments, reconstituable dry powder formulations described in this disclosure are useful for improved therapeutic efficacy, including improved delivery of mRNA to the cells.

In some embodiments, the reconstituable dry powder formulations have improved delivery of mRNA to various tissues compared to liquid controls. In some embodiments, the formulations described in this disclosure have improved delivery when used orally, mucosally, intravenously, intramuscularly, subcutaneously, transdermally or buccally. In some embodiments, the formulations described in this disclosure have improved delivery when used in delivery routes that are suitable for the present disclosure.

In some embodiments, a reconstitutable dry powder formulation described herein provides comparable delivery efficacy as compared to an equivalent non-dry powder formulation. In some embodiments, the delivery efficacy does not appreciably change during storage of a reconstituable dry powder formulation, including any exemplary storage, duration, and/or temperature described herein. In some embodiments, the delivery efficacy of a dry powder formulation does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following storage, including according to any exemplary storage, duration, and/or temperature as described herein.

In some embodiments, reconstitutable dry powder formulation described herein provides comparable delivery efficacy as compared to the dry powder formulation prior to reconstitution. In some embodiments, the delivery efficacy of a reconstituable dry powder formulation does not change by more than about 1%, 2%, 2.5%, 4%, 5%, 7.5%, 10%, 12.5%, 15%, 18.5%, 20%, 25%, 30%, 35%, 40% or 50% (e.g., no more than about 30%, 25%, 20%, 15%, 10%, or 5%), including all values and ranges therebetween, following reconstitution, including according to any method as described herein.

g. Sugar-Lipid Ratios

Reconstitutable dry powder formulations described herein can have an optimal particle size for tissue specific delivery.

In some embodiments, the reconstitutable dry powder formulation comprising trehalose is used for pulmonary delivery. In some embodiments, the ratio of concentration of trehalose to total lipid concentration is greater than 1. In some embodiments, the ratio of concentration of trehalose to total lipid concentration is greater than 2. In some embodiments, the ratio of concentration of trehalose to total lipid concentration is greater than 3. In some embodiments, the ratio of concentration of trehalose to total lipid concentration is greater than 4. In some embodiments, the ratio of concentration of trehalose to total lipid concentration is greater than 5. In some embodiments, the ratio of concentration of trehalose to total lipid concentration is greater than 6. In some embodiments, the ratio of concentration of trehalose to total lipid concentration is greater than 7. In some embodiments, the ratio of concentration of trehalose to total lipid concentration is greater than 8. In some embodiments, the ratio of concentration of trehalose to total lipid concentration is greater than 9. In some embodiments, the ratio of concentration of trehalose to total lipid concentration is greater than 10. In some embodiments, the ratio of concentration of trehalose to total lipid concentration is greater than 11. In some embodiments, the ratio of concentration of trehalose to total lipid concentration is greater than 12. In some embodiments, the ratio of concentration of trehalose to total lipid concentration is greater than 13. In some embodiments, the ratio of concentration of trehalose to total lipid concentration is greater than 14. In some embodiments, the ratio of concentration of trehalose to total lipid concentration is greater than 15. In some embodiments, the ratio of concentration of trehalose to total lipid concentration is greater than 16. In some embodiments, the ratio of concentration of trehalose to total lipid concentration is greater than 17. In some embodiments, the ratio of concentration of trehalose to total lipid concentration is greater than 18. In some embodiments, the ratio of concentration of trehalose to total lipid concentration is greater than 19. In some embodiments, the ratio of concentration of trehalose to total lipid concentration is greater than 20.

h. Encapsulation Efficiencies

A reconstitutable dry powder formulation as described herein can provide beneficial effects in the encapsulation efficiencies of the LNPs of the formulation, including but not limited to the exemplary embodiments described herein.

In some embodiments, the reconstitutable dry powder formulation comprising sugar or sugar alcohol as described herein comprises higher encapsulation efficiency compared to liquid controls or dry powders without sugar or sugar alcohol. In some embodiments, the encapsulation efficiency is greater than 60%. In some embodiments, the encapsulation efficiency is greater than 61%. In some embodiments, the encapsulation efficiency is greater than 62%. In some embodiments, the encapsulation efficiency is greater than 63%. In some embodiments, the encapsulation efficiency is greater than 64%. In some embodiments, the encapsulation efficiency is greater than 65%. In some embodiments, the encapsulation efficiency is greater than 66%. In some embodiments, the encapsulation efficiency is greater than 67%. In some embodiments, the encapsulation efficiency is greater than 68%. In some embodiments, the encapsulation efficiency is greater than 69%. In some embodiments, the encapsulation efficiency is greater than 70%. In some embodiments, the encapsulation efficiency is greater than 71%. In some embodiments, the encapsulation efficiency is greater than 72%. In some embodiments, the encapsulation efficiency is greater than 73%. In some embodiments, the encapsulation efficiency is greater than 74%. In some embodiments, the encapsulation efficiency is greater than 75%. In some embodiments, the encapsulation efficiency is greater than 76%. In some embodiments, the encapsulation efficiency is greater than 77%. In some embodiments, the encapsulation efficiency is greater than 78%. In some embodiments, the encapsulation efficiency is greater than 79%. In some embodiments, the encapsulation efficiency is greater than 80%. In some embodiments, the encapsulation efficiency is greater than 81%. In some embodiments, the encapsulation efficiency is greater than 82%. In some embodiments, the encapsulation efficiency is greater than 83%. In some embodiments, the encapsulation efficiency is greater than 84%. In some embodiments, the encapsulation efficiency is greater than 85%. In some embodiments, the encapsulation efficiency is greater than 86%. In some embodiments, the encapsulation efficiency is greater than 87%. In some embodiments, the encapsulation efficiency is greater than 88%. In some embodiments, the encapsulation efficiency is greater than 89%. In some embodiments, the encapsulation efficiency is greater than 90%. In some embodiments, the encapsulation efficiency is greater than 91%. In some embodiments, the encapsulation efficiency is greater than 92%. In some embodiments, the encapsulation efficiency is greater than 93%. In some embodiments, the encapsulation efficiency is greater than 94%. In some embodiments, the encapsulation efficiency is greater than 95%. In some embodiments, the encapsulation efficiency is greater than 96%. In some embodiments, the encapsulation efficiency is greater than 97%. In some embodiments, the encapsulation efficiency is greater than 98%. In some embodiments, the encapsulation efficiency is greater than 99%.

In some embodiments, the particle size (e.g., mean particle size) of the dry powders comprised in the reconstitutable dry powder formulation comprising sugar or sugar alcohol as described herein is less than 3 μm. In some embodiments, the particle size (e.g., mean particle size) of the reconstitutable dry powder is less than 4 μm. In some embodiments, the particle size (e.g., mean particle size) of the reconstitutable dry powder is less than 5 μm. In some embodiments, the particle size (e.g., mean particle size) of the reconstitutable dry powder is less than 6 μm. In some embodiments, the particle size (e.g., mean particle size) of the reconstitutable dry powder is less than 7 μm. In some embodiments, the particle size (e.g., mean particle size) of the reconstitutable dry powder is less than 8 μm. In some embodiments, the particle size (e.g., mean particle size) of the reconstitutable dry powder is less than 9 μm. In some embodiments, the particle size (e.g., mean particle size) of the reconstitutable dry powder is less than 10 μm.

In some embodiments, a reconstitutable dry powder formulation described herein exhibits an enhanced (e.g., increased) ability to transfect one or more target cells. Accordingly, also provided herein are methods of transfecting one or more target cells. Such methods generally comprise the step of contacting the one or more target cells with, for example, a dry powder formulation as described herein such that the one or more target cells are transfected with the materials encapsulated therein (e.g., one or more polynucleotides or mRNA). For example, and as further described herein, a dry powder formulation can be beneficial for needle-free (i.e., non-invasive) routes of administration such as, but not limited to, delivery via mucosal surfaces.

### Exemplary Features of Reconstituted Dry Powder Formulations Described Herein

Reconstitutable dry powder formulations as described herein provide beneficial properties following reconstitution to the desired mRNA-LNP formulation, including but not limited to the exemplary beneficial features described herein.

In some embodiments, a reconstitutable dry powder formulation as described herein can maintain desirable features following reconstitution including, but not limited to beneficial stability (e.g., as determined, for example, with reference to the particle size (e.g., mean particle size) of the reconstituted lipid nanoparticles comprising such composition). In some embodiments, reconstitution of a dry powder formulation described herein does not appreciably change or alter the particle size (e.g., mean particle size) and/or encapsulation efficiency of the lipid nanoparticles following reconstitution. In some embodiments, reconstitution of a dry powder formulation described herein does not appreciably change or alter the therapeutic efficacy of the mRNA (e.g., the integrity and/or activity of the mRNA) following reconstitution.

a. Lack of Aggregation

For example, a reconstituted dry powder formulation as described herein can provide beneficial effects in the stability of the formulation, by for example, minimizing aggregation, including but not limited to the exemplary embodiments described herein.

In some embodiments, disclosed herein are dry powder compositions wherein upon reconstitution (e.g., according to methods described herein) the lipid nanoparticles do not flocculate or aggregate, or alternatively demonstrated limited or negligible flocculation or aggregation (e.g., a determined by the particle size (e.g., mean particle size) of the reconstituted lipid nanoparticles). Accordingly, in some embodiments, upon reconstitution of a dry powder formulation as described herein, the lipid nanoparticles have a geometric particle size distribution Dv50 of less than about 500 nm (e.g., less than about 300 nm, 200 nm, 150 nm, 125 nm, 120 nm, 100 nm, 75 nm, 50 nm, 25 nm, or smaller, including all values and ranges therebetween). Similarly, in some embodiments, upon reconstitution of a dry powder formulation as described herein, the lipid nanoparticles have a geometric particle size distribution Dv90 of less than about 750 nm (e.g., less than about 700 nm, 500 nm, 300 nm, 200 nm, 150 nm, 125 nm, 100 nm, 75 nm, 50 nm, 25 nm, or smaller, including all values and ranges therebetween).

In some embodiments, upon reconstitution with an appropriate rehydration media, the reconstituted dry powder formulation demonstrates pharmacological or biological activity comparable with that observed prior to dry powder formulation. For example, in some embodiments, the pharmacological or biological activity of an encapsulated polynucleotide in a reconstituted formulation is equivalent to that observed prior to dry powder formulation of the composition, or alternatively demonstrates a negligible reduction in pharmacological or biological activity (e.g., less than about a 1%, 2%, 2.5%, 4%, 5%, 7.5%, 10%, 12.5%, 15%, 18.5%, 20%, 25%, 30%, 35%, 40%, 50%, including all values and ranges therebetween, reduction in the biological or pharmacological activity of an encapsulated polynucleotide).

Also provided herein are methods of transfecting one or more target cells. In some embodiments, a reconstituted composition (e.g., lipid nanoparticles) of the present disclosure exhibits an enhanced (e.g., increased) ability to transfect one or more target cells. Such methods generally comprise the step of contacting the one or more target cells with a reconstituted dry powder formulation as described herein such that the one or more target cells are transfected with the materials encapsulated therein (e.g., one or more polynucleotides or mRNA).

b. Reconstitution solvents

In some embodiments, the reconstitutable dry powder product as disclosed herein comprising mRNA-nanoparticle and excipients is reconstituted with a solvent. The reconstitution of the dry powder product provides advantages for parenteral administration. In some embodiments, the solvent is water.

In some embodiments, the solvent is a buffer. The buffers or pH-adjusting agent in emulsion compositions, is used to adjust the pH to a desirable range. Exemplary buffers include but are not limited to phosphate buffer, citrate buffer, tris buffer, carbonate buffer, succinate buffer, maleate buffer and borate buffer. In some embodiments, the buffer is selected from the group of phosphate buffered saline (PBS), modified PBS and citrate buffer. In some embodiments, the buffer can be any buffer that is suitable for the formulations and methods described in the present disclosure.

In some embodiments, the reconstitution of the dry powder product involves, reconstitution in a buffer directly. In some embodiments, the reconstitution of the dry powder product in a buffer comprises the steps: (1) reconstitution with water and (2) reconstitution with a buffer.

### C. Stability

A reconstituted dry powder formulation as described herein can provide beneficial effects in the stability of the formulation, including but not limited to the exemplary embodiments described herein.

In some embodiments, the reconstituted dry powder formulation comprising sugar or sugar alcohol as described herein is stable for at least 1 week. In some embodiments, the reconstituted dry powder formulation is stable for at least 2 weeks. In some embodiments, the reconstituted dry powder formulation is stable for at least 3 weeks. In some embodiments, the reconstituted dry powder formulation is stable for at least 4 weeks. In some embodiments, the reconstituted dry powder formulation is stable for at least 1 month. In some embodiments, the reconstituted dry powder formulation is stable for at least 2 months. In some embodiments, the reconstituted dry powder formulation is stable for at least 3 months. In some embodiments, the reconstituted dry powder formulation is stable for at least 4 months. In some embodiments, the reconstituted dry powder formulation is stable for at least 5 months. In some embodiments, the reconstituted dry powder formulation is stable for at least 6 months. In some embodiments, the reconstituted dry powder formulation is stable for at least 7 months. In some embodiments, the reconstituted dry powder formulation is stable for at least 8 months. In some embodiments, the reconstituted dry powder formulation is stable for at least 9 months. In some embodiments, the reconstituted dry powder formulation is stable for at least 10 months. In some embodiments, the reconstituted dry powder formulation is stable for at least 11 months. In some embodiments, the reconstituted dry powder formulation is stable for at least 1 year. In some embodiments, the reconstituted dry powder formulation is stable for about 1 year or more.

In some embodiments, the stability of the reconstituted dry powder formulation comprising sugar or sugar alcohol is increased by at least 1 week compared to liquid controls. In some embodiments, the stability of the reconstituted dry powder formulation is increased by at least 2 weeks compared to liquid controls. In some embodiments, the stability of the reconstituted dry powder formulation is increased by at least 3 weeks compared to liquid controls. In some embodiments, the stability of the reconstituted dry powder formulation is increased by at least 4 weeks compared to liquid controls. In some embodiments, the stability of the dry powder is increased by at least 1 month compared to liquid controls. In some embodiments, the stability of the reconstituted dry powder formulation is increased by at least 2 months compared to liquid controls. In some embodiments, the stability of the reconstituted dry powder formulation is increased by at least 3 months compared to liquid controls. In some embodiments, the stability of the reconstituted dry powder formulation is increased by at least 4 months compared to liquid controls. In some embodiments, the stability of the reconstituted dry powder formulation is increased by at least 5 months compared to liquid controls. In some embodiments, the stability of the reconstituted dry powder formulation is increased by at least 6 months compared to liquid controls. In some embodiments, the stability of the reconstituted dry powder formulation is increased by at least 7 months compared to liquid controls. In some embodiments, the stability of the reconstituted dry powder formulation is increased by at least 8 months compared to liquid controls. In the stability of the reconstituted dry powder formulation is increased by at least 9 months compared to liquid controls. In some embodiments, the stability of the reconstituted dry powder formulation is increased by at least 10 months compared to liquid controls. In some embodiments, the stability of the reconstituted dry powder formulation is increased by at least 11 months compared to liquid controls. In some embodiments, the stability of the reconstituted dry powder formulation is increased by at least 1 year compared to liquid controls. In some embodiments, the stability of the reconstituted dry powder formulation is increased by 1 year or more compared to liquid controls.

In some embodiments, the reconstituted dry powder formulation comprising sugar or sugar alcohol comprises better thermostability when compared to liquid controls. In some embodiments, the reconstituted dry powder formulation is thermostable at 4° C. for at least 1 week compared to liquid controls. In some embodiments, the reconstituted dry powder formulation is stable at 4° C. for at least 2 weeks. In some embodiments, the reconstituted dry powder formulation is thermostable at 4° C. for at least 3 weeks compared to liquid controls. In some embodiments, the reconstituted dry powder formulation is thermostable at 4° C. for at least 4 weeks compared to liquid controls. In some embodiments, the reconstituted dry powder formulation is thermostable at 4° C. for 1 month compared to liquid controls. In some embodiments, the reconstituted dry powder formulation is thermostable at 4° C. for at least 2 months compared to liquid controls. In some embodiments, the reconstituted dry powder formulation is thermostable at 4° C. for at least 3 months compared to liquid controls. In some embodiments, the reconstituted dry powder formulation is thermostable at 4° C. for at least 4 months compared to liquid controls. In some embodiments, the reconstituted dry powder formulation is thermostable at 4° C. for at least 5 months compared to liquid controls. In some embodiments, the reconstituted dry powder formulation is thermostable at 4° C. for at least 6 months compared to liquid controls. In some embodiments, the reconstituted dry powder formulation is thermostable at 4° C. for at least 7 months compared to liquid controls. In some embodiments, the reconstituted dry powder formulation is thermostable at 4° C. for at least 8 months compared to liquid controls. In some embodiments, the reconstituted dry powder formulation is thermostable at 4° C. for at least 9 months compared to liquid controls. In some embodiments, the reconstituted dry powder formulation is thermostable at 4° C. for at least 10 months compared to liquid controls. In some embodiments, the reconstituted dry powder formulation is thermostable at 4° C. for at least 11 months compared to liquid controls. In some embodiments, the reconstituted dry powder formulation is thermostable at 4° C. for at least 1 year compared to liquid controls. In some embodiments, the reconstituted dry powder formulation is thermostable at 4° C. for more than 1 year compared to liquid controls.

In some embodiments, the reconstituted dry powder formulation comprising sugar or sugar alcohol is stable at 25° C. for at least 1 week compared to liquid controls. In some embodiments, the reconstituted dry powder formulation is stable at 25° C. for at least 2 weeks compared to liquid controls. In some embodiments, the reconstituted dry powder formulation is stable at 25° C. for at least 3 weeks compared to liquid controls. In some embodiments, the reconstituted dry powder formulation is stable at 25° C. for at least 4 weeks compared to liquid controls. In some embodiments, the reconstituted dry powder formulation is stable at 25° C. for 1 month compared to liquid controls. In some embodiments, the reconstituted dry powder formulation is stable at 25° C. for at least 2 months compared to liquid controls. In some embodiments, the reconstituted dry powder formulation is stable at 25° C. for at least 3 months compared to liquid controls. In some embodiments, the reconstituted dry powder formulation is stable at 25° C. for at least 4 months compared to liquid controls. In some embodiments, the reconstituted dry powder formulation is stable at 25° C. for at least 5 months compared to liquid controls. In some embodiments, the reconstituted dry powder formulation is stable at 25° C. for at least 6 months compared to liquid controls. In some embodiments, the reconstituted dry powder formulation is stable at 25° C. for at least 7 months compared to liquid controls. In some embodiments, the reconstituted dry powder formulation is stable at 25° C. for at least 8 months compared to liquid controls. In some embodiments, the reconstituted dry powder formulation is stable at 25° C. for at least 9 months compared to liquid controls. In some embodiments, the reconstituted dry powder formulation is stable at 25° C. for at least 10 months compared to liquid controls. In some embodiments, the reconstituted dry powder formulation is stable at 25° C. for at least 11 months compared to liquid controls. In some embodiments, the reconstituted dry powder formulation is stable at 25° C. for at least 1 year. In some embodiments, the reconstituted dry powder formulation is stable at 25° C. for more than 1 year.

In some embodiments, the reconstituted dry powder formulation comprising sugar or sugar alcohol is stable at −20° C. for at least 1 week compared to liquid controls. In some embodiments, the reconstituted dry powder formulation is stable at −20° C. for at least 2 weeks compared to liquid controls. In some embodiments, the reconstituted dry powder formulation is stable at −20° C. for at least 3 weeks compared to liquid controls. In some embodiments, the reconstituted dry powder formulation is stable at −20° C. for at least 4 weeks compared to liquid controls. In some embodiments, the reconstituted dry powder formulation is stable at −20° C. for 1 month compared to liquid controls. In some embodiments, the reconstituted dry powder formulation is stable at −20° C. for at least 2 months compared to liquid controls. In some embodiments, the reconstituted dry powder formulation is stable at −20° C. for at least 3 months compared to liquid controls. In some embodiments, the reconstituted dry powder formulation is stable at −20° C. for at least 4 months compared to liquid controls. In some embodiments, the reconstituted dry powder formulation is stable at −20° C. for at least 5 months compared to liquid controls. In some embodiments, the reconstituted dry powder formulation is stable at −20° C. for at least 6 months compared to liquid controls. In some embodiments, the reconstituted dry powder formulation is stable at −20° C. for at least 7 months compared to liquid controls. In some embodiments, the reconstituted dry powder formulation is stable at −20° C. for at least 8 months compared to liquid controls. In some embodiments, the reconstituted dry powder formulation is stable at −20° C. for at least 9 months compared to liquid controls. In some embodiments, the reconstituted dry powder formulation is stable at −20° C. for at least 10 months compared to liquid controls. In some embodiments, the reconstituted dry powder formulation is stable at −20° C. for at least 11 months compared to liquid controls. In some embodiments, the reconstituted dry powder formulation is stable at −20° C. for at least 1 year compared to liquid controls. In some embodiments, the reconstituted dry powder formulation is stable at −20° C. for more than 1 year compared to liquid controls.

In some embodiments, the reconstituted dry powder formulation demonstrates a negligible reduction in pharmacological or biological activity (e.g., less than about a 1%, 2%, 2.5%, 4%, 5%, 7.5%, 10%, 12.5%, 15%, 18.5%, 20%, 25%, 30%, 35%, 40%, 50%, including all values and ranges therebetween, reduction in the biological or pharmacological activity of an encapsulated polynucleotide) (e.g., as compared to prior to dry powder formulation).

In some embodiments, the reconstituted dry powder formulation described herein provides comparable stability as compared to an equivalent non-dry powder formulation. In some embodiments, the stability does not appreciably change during storage of a dry powder formulation, including any exemplary storage, duration, and/or temperature described herein. In some embodiments, the stability of a reconstituted dry powder formulation does not change by more than about 1%, 2%, 2.5%, 4%, 5%, 7.5%, 10%, 12.5%, 15%, 18.5%, 20%, 25%, 30%, 35%, 40% or 50%, including all values and ranges therebetween, (e.g., no more than about 30%, 25%, 20%, 15%, 10%, or 5%, including all values and ranges therebetween) following storage, including according to any exemplary storage, duration, and/or temperature as described herein.

In some embodiments, the reconstituted dry powder formulation described herein provides comparable stability as compared to the reconstituted dry powder formulation prior to reconstitution. In some embodiments, the stability of a reconstituted dry powder formulation does not change by more than about 1%, 2%, 2.5%, 4%, 5%, 7.5%, 10%, 12.5%, 15%, 18.5%, 20%, 25%, 30%, 35%, 40%, 50%, including all values and ranges therebetween, (e.g., no more than about 30%, 25%, 20%, 15%, 10%, or 5%, including all values and ranges therebetween) following reconstitution, including according to any method as described herein.

d. Storage

A reconstituted dry powder formulation as described herein can provide beneficial effects in the storage and/or storage stability, including but not limited to the exemplary embodiments described herein.

In some embodiments, a dry powder formulation as described herein can have an improved duration of storage (including, for example, at relatively elevated temperatures that do not require extreme cold storage). In some embodiments, a reconstituted dry powder formulation as described herein is also characterized by their improved storage properties. For example, some embodiments, a reconstituted dry powder formulation may be stored under refrigeration and remain stable (e.g., as demonstrated by minimal or no losses in their intended pharmaceutical or biological activity) for extended periods of time (e.g., stable for at least about 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer upon storage at temperatures such as those described herein, including at about 4° C. or −20° C.). In some embodiments, a reconstitutable dry powder formulation may be stored without refrigeration and remain stable for extended periods of time (e.g., stable for at least about 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer upon storage at ambient temperatures, including at about 25° C.).

In some embodiments, a reconstituted dry powder formulation described herein provides comparable duration of storage as compared to an equivalent non-dry powder formulation. In some embodiments, the duration of storage does not appreciably change during storage of the reconstituted dry powder formulation, including any exemplary storage, duration, and/or temperature described herein. In some embodiments, the duration of storage of the reconstituted dry powder formulation does not change by more than about 1%, 2%, 2.5%, 4%, 5%, 7.5%, 10%, 12.5%, 15%, 18.5%, 20%, 25%, 30%, 35%, 40%, 50%, including values and ranges therebetween, (e.g., no more than about 30%, 25%, 20%, 15%, 10%, or 5%, including all values and ranges therebetween) following storage, including according to any exemplary storage, duration, and/or temperature as described herein.

In some embodiments, the reconstituted dry powder formulation described herein provides comparable duration of storage as compared to the dry powder formulation prior to reconstitution. In some embodiments, the duration of storage of a reconstituted dry powder formulation does not change by more than about 1%, 2%, 2.5%, 4%, 5%, 7.5%, 10%, 12.5%, 15%, 18.5%, 20%, 25%, 30%, 35%, 40%, 50%, including ranges and values therebetween, (e.g., no more than about 30%, 25%, 20%, 15%, 10%, or 5%, including all values and ranges therebetween) following reconstitution, including according to any method as described herein.

e. Particle Size

A reconstituted dry powder formulation as described herein can provide beneficial effects in the particle size (e.g., mean particle size) of the LNPs of the formulation, including but not limited to the exemplary embodiments described herein.

In some embodiments, the particle size of the reconstituted dry powder formulation comprising sugar or sugar alcohol as excipients is less than 80 nm. In some embodiments, the particle size of the reconstituted dry powder formulation comprising sugar or sugar alcohol as excipients is less than 81 nm. In some embodiments, the particle size of the reconstituted dry powder formulation comprising sugar or sugar alcohol as excipients is less than 82 nm. In some embodiments, the particle size of the reconstituted dry powder formulation comprising sugar or sugar alcohol as excipients is less than 83 nm. In some embodiments, the particle size of the reconstituted dry powder formulation comprising sugar or sugar alcohol as excipients is less than 84 nm. In some embodiments, the particle size of the reconstituted dry powder formulation comprising sugar or sugar alcohol as excipients is less than 85 nm. In some embodiments, the particle size of the reconstituted dry powder formulation comprising sugar or sugar alcohol as excipients is less than 86 nm. In some embodiments, the particle size of the reconstituted dry powder formulation comprising sugar or sugar alcohol as excipients is less than 87 nm. In some embodiments, the particle size of the reconstituted dry powder formulation comprising sugar or sugar alcohol as excipients is less than 88 nm. In some embodiments, the particle size of the reconstituted dry powder formulation comprising sugar or sugar alcohol as excipients is less than 89 nm. In some embodiments, the particle size of the reconstituted dry powder formulation comprising sugar or sugar alcohol as excipients is less than 90 nm. In some embodiments, the particle size of the reconstituted dry powder formulation comprising sugar or sugar alcohol as excipients is less than 91 nm. In some embodiments, the particle size of the reconstituted dry powder formulation comprising sugar or sugar alcohol as excipients is less than 92 nm. In some embodiments, the particle size of the reconstituted dry powder formulation comprising sugar or sugar alcohol as excipients is less than 93 nm. In some embodiments, the particle size of the reconstituted dry powder formulation comprising sugar or sugar alcohol as excipients is less than 94 nm. In some embodiments, the particle size of the reconstituted dry powder formulation comprising sugar or sugar alcohol as excipients is less than 95 nm. In some embodiments, the particle size of the reconstituted dry powder formulation comprising sugar or sugar alcohol as excipients is less than 96 nm. In some embodiments, the particle size of the reconstituted dry powder formulation comprising sugar or sugar alcohol as excipients is less than 97 nm. In some embodiments, the particle size of the reconstituted dry powder formulation comprising sugar or sugar alcohol as excipients is less than 98 nm. In some embodiments, the particle size of the reconstituted dry powder formulation comprising sugar or sugar alcohol as excipients is less than 99 nm. In some embodiments, the particle size of the reconstituted dry powder formulation comprising sugar or sugar alcohol as excipients is less than 100 nm. In some embodiments, the particle size of the reconstituted dry powder formulation comprising sugar or sugar alcohol as excipients is less than 101 nm. In some embodiments, the particle size of the reconstituted dry powder formulation comprising sugar or sugar alcohol as excipients is less than 102 nm. In some embodiments, the particle size of the reconstituted dry powder formulation comprising sugar or sugar alcohol as excipients is less than 103 nm. In some embodiments, the particle size of the reconstituted dry powder formulation comprising sugar or sugar alcohol as excipients is less than 104 nm. In some embodiments, the particle size of the reconstituted dry powder formulation comprising sugar or sugar alcohol as excipients is less than 105 nm. In some embodiments, the particle size of the reconstituted dry powder formulation comprising sugar or sugar alcohol as excipients is less than 106 nm. In some embodiments, the particle size of the reconstituted dry powder formulation comprising sugar or sugar alcohol as excipients is less than 107 nm. In some embodiments, the particle size of the reconstituted dry powder formulation comprising sugar or sugar alcohol as excipients is less than 108 nm. In some embodiments, the particle size of the reconstituted dry powder formulation comprising sugar or sugar alcohol as excipients is less than 109 nm. In some embodiments, the particle size of the reconstituted dry powder formulation comprising sugar or sugar alcohol as excipients is less than 110 nm. In some embodiments, the particle size of the reconstituted dry powder formulation comprising sugar or sugar alcohol as excipients is less than 111 nm. In some embodiments, the particle size of the reconstituted dry powder formulation comprising sugar or sugar alcohol as excipients is less than 112 nm. In some embodiments, the particle size of the reconstituted dry powder formulation comprising sugar or sugar alcohol as excipients is less than 113 nm. In some embodiments, the particle size of the reconstituted dry powder formulation comprising sugar or sugar alcohol as excipients is less than 114 nm. In some embodiments, the particle size of the reconstituted dry powder formulation comprising sugar or sugar alcohol as excipients, is less than 115 nm. In some embodiments, the particle size of the reconstituted dry powder formulation comprising sugar or sugar alcohol as excipients is less than 116 nm. In some embodiments, the particle size of the reconstituted dry powder formulation comprising sugar or sugar alcohol as excipients is less than 117 nm. In some embodiments, the particle size of the reconstituted dry powder formulation comprising sugar or sugar alcohol as excipients is less than 118 nm. In some embodiments, the particle size of the reconstituted dry powder formulation comprising sugar or sugar alcohol as excipients is less than 119 nm. In some embodiments, the particle size of the reconstituted dry powder formulation comprising sugar or sugar alcohol as excipients is less than 120 nm. In some embodiments, the particle size of the reconstituted dry powder formulation comprising sugar or sugar alcohol as excipients is less than 121 nm. In some embodiments, the particle size of the reconstituted dry powder formulation comprising sugar or sugar alcohol as excipients is less than 122 nm. In some embodiments, the particle size of the reconstituted dry powder formulation comprising sugar or sugar alcohol as excipients is less than 123 nm. In some embodiments, the particle size of the reconstituted dry powder formulation comprising sugar or sugar alcohol as excipients is less than 124 nm. In some embodiments, the particle size of the reconstituted dry powder formulation comprising sugar or sugar alcohol as excipients is less than 125 nm. In some embodiments, the particle size of the reconstituted dry powder formulation comprising sugar or sugar alcohol as excipients is less than 126 nm. In some embodiments, the particle size of the reconstituted dry powder formulation comprising sugar or sugar alcohol as excipients is less than 127 nm. In some embodiments, the particle size of the reconstituted dry powder formulation comprising sugar or sugar alcohol as excipients is less than 128 nm. In some embodiments, the particle size of the reconstituted dry powder formulation comprising sugar or sugar alcohol as excipients is less than 129 nm. In some embodiments, the particle size of the reconstituted dry powder formulation comprising sugar or sugar alcohol as excipients is less than 130 nm. In some embodiments, the particle size of the reconstituted dry powder formulation comprising sugar or sugar alcohol as excipients is less than 131 nm. In some embodiments, the particle size of the reconstituted dry powder formulation comprising sugar or sugar alcohol as excipients is less than 132 nm. In some embodiments, the particle size of the reconstituted dry powder formulation comprising sugar or sugar alcohol as excipients is less than 133 nm. In some embodiments, the particle size of the reconstituted dry powder formulation comprising sugar or sugar alcohol as excipients is less than 134 nm. In some embodiments, the particle size of the reconstituted dry powder formulation comprising sugar or sugar alcohol as excipients is less than 135 nm. In some embodiments, the particle size of the reconstituted dry powder formulation comprising sugar or sugar alcohol as excipients is less than 136 nm. In some embodiments, the particle size of the reconstituted dry powder formulation comprising sugar or sugar alcohol as excipients is less than 137 nm. In some embodiments, the particle size of the reconstituted dry powder formulation comprising sugar or sugar alcohol as excipients is less than 138 nm. In some embodiments, the particle size of the reconstituted dry powder formulation comprising sugar or sugar alcohol as excipients is less than 139 nm. In some embodiments, the particle size of the reconstituted dry powder formulation comprising sugar or sugar alcohol as excipients is less than 140 nm. In some embodiments, the particle size of the reconstituted dry powder formulation comprising sugar or sugar alcohol as excipients is less than 141 nm. In some embodiments, the particle size of the reconstituted dry powder formulation comprising sugar or sugar alcohol as excipients is less than 142 nm. In some embodiments, the particle size of the reconstituted dry powder formulation comprising sugar or sugar alcohol as excipients is less than 143 nm. In some embodiments, the particle size of the reconstituted dry powder formulation comprising sugar or sugar alcohol as excipients is less than 144 nm. In some embodiments, the particle size of the reconstituted dry powder formulation comprising sugar or sugar alcohol as excipients is less than 145 nm. In some embodiments, the particle size of the reconstituted dry powder formulation comprising sugar or sugar alcohol as excipients is less than 146 nm. In some embodiments, the particle size of the reconstituted dry powder formulation comprising sugar or sugar alcohol as excipients is less than 147 nm. In some embodiments, the particle size of the reconstituted dry powder formulation comprising sugar or sugar alcohol as excipients is less than 148 nm. In some embodiments, the particle size of the reconstituted dry powder formulation comprising sugar or sugar alcohol as excipients is less than 149 nm. In some embodiments, the particle size of the reconstituted dry powder formulation comprising sugar or sugar alcohol as excipients is less than 150 nm.

In some embodiments, the “particle size of the reconstituted dry powder formulation” described herein refers to the mean particle size of the LNPs in the reconstituted dry powder formulation. Accordingly, in some embodiments, the mean particle size of the LNPs in the reconstituted dry powder formulation comprising sugar or sugar alcohol as excipients is less than 150 nm, such as less than 145 nm, less than 140 nm, less than 135 nm, less than 130 nm, less than 125 nm, less than 120 nm, less than 115 nm, less than 110, less than 105 nm, less than 100 nm, less than 95 nm, less than 90 nm, less than 85 nm, or less than 80 nm, including all values and ranges therebetween.

In some embodiments, a reconstituted dry powder formulation described herein provides comparable particle size (e.g., mean particle size of the LNPs in the reconstituted formulation) as compared to an equivalent non-dry powder formulation. In some embodiments, the particle size (e.g., mean particle size of the reconstituted LNPs) does not appreciably change during storage of the reconstituted dry powder formulation, including any exemplary storage, duration, and/or temperature described herein. In some embodiments, the particle size of the reconstituted dry powder formulation (e.g., mean particle size of the LNPs in the reconstituted formulation) does not change by more than about 1%, 2%, 2.5%, 4%, 5%, 7.5%, 10%, 12.5%, 15%, 18.5%, 20%, 25%, 30%, 35%, 40%, 50%, including all values and ranges therebetween, (e.g., no more than about 30%, 25%, 20%, 15%, 10%, or 5%, including all values and ranges therebetween) following storage, including according to any exemplary storage, duration, and/or temperature as described herein.

In some embodiments, the reconstituted dry powder formulation described herein provides comparable particle size (e.g., mean particle size of the LNPs in the reconstituted formulation) as compared to the reconstituted dry powder formulation prior to reconstitution. In some embodiments, the particle size of a reconstituted dry powder formulation (e.g., mean particle size of the LNPs in the reconstituted formulation) does not change by more than about 1%, 2%, 2.5%, 4%, 5%, 7.5%, 10%, 12.5%, 15%, 18.5%, 20%, 25%, 30%, 35%, 40%, 50%, including all values and ranges therebetween, (e.g., no more than about 30%, 25%, 20%, 15%, 10%, or 5%, including all values and ranges therebetween) following reconstitution, including according to any method as described herein.

f. mRNA Integrity

The reconstituted dry powder formulation as described herein can provide beneficial effects in the integrity of the mRNA cargo, including but not limited to the exemplary embodiments described herein.

In some embodiments, the mRNA maintains an integrity of about 60%, 65%, 70%, 75%, 80%, 85%, 90%, 92%, 94%, 96%, 98% or greater, including any values and subranges therebetween, after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 60% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 61% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 62% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 63% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 64% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 65% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 66% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 67% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 68% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 69% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 70% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36months or longer. In some embodiments, the mRNA maintains an integrity of 71% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 72% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 73% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 74% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 75% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 76% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 77% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 78% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 79% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 80% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 81% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 82% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 83% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 84% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 85% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 86% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 87% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 88% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 89% or greater after storage for 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months or longer. In some embodiments, the mRNA maintains an integrity of 90% or greater for 1, 2, 3, 4, 5, 6, 9, 12, 18, 24, 36 months or longer.

In some embodiments, the mRNA maintains an integrity of about 60%, 65%, 70%, 75%, 80%, 85%, 90%, 92%, 94%, 96%, 98% or greater, including any values and subranges therebetween, after storage at 4° C. for at least 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36 months. In some embodiments, the mRNA maintains an integrity of 60% or greater after storage at 4° C. for at least six months. In some embodiments, the mRNA maintains an integrity of 61% or greater after storage at 4° C. for at least six months. In some embodiments, the mRNA maintains an integrity of 62% or greater after storage at 4° C. for at least six months. In some embodiments, the mRNA maintains an integrity of 63% or greater after storage at 4° C. for at least six months. In some embodiments, the mRNA maintains an integrity of 64% or greater after storage at 4° C. for at least six months. In some embodiments, the mRNA maintains an integrity of 65% or greater after storage at 4° C. for at least six months. In some embodiments, the mRNA maintains an integrity of 66% or greater after storage at 4° C. for at least six months. In some embodiments, the mRNA maintains an integrity of 67% or greater after storage at 4° C. for at least six months. In some embodiments, the mRNA maintains an integrity of 68% or greater after storage at 4° C. for at least six months. In some embodiments, the mRNA maintains an integrity of 69% or greater after storage at 4° C. for at least six months. In some embodiments, the mRNA maintains an integrity of 70% or greater after storage at 4° C. for at least six months. In some embodiments, the mRNA maintains an integrity of 71% or greater after storage at 4° C. for at least six months. In some embodiments, the mRNA maintains an integrity of 72% or greater after storage at 4° C. for at least six months. In some embodiments, the mRNA maintains an integrity of 73% or greater after storage at 4° C. for at least six months. In some embodiments, the mRNA maintains an integrity of 74% or greater after storage at 4° C. for at least six months. In some embodiments, the mRNA maintains an integrity of 75% or greater after storage at 4° C. for at least six months. In some embodiments, the mRNA maintains an integrity of 76% or greater after storage at 4° C. for at least six months. In some embodiments, the mRNA maintains an integrity of 77% or greater after storage at 4° C. for at least six months. In some embodiments, the mRNA maintains an integrity of 78% or greater after storage at 4° C. for at least six months. In some embodiments, the mRNA maintains an integrity of 79% or greater after storage at 4° C. for at least six months. In some embodiments, the mRNA maintains an integrity of 80% or greater after storage at 4° C. for at least six months. In some embodiments, the mRNA maintains an integrity of 81% or greater after storage at 4° C. for at least six months. In some embodiments, the mRNA maintains an integrity of 82% or greater after storage at 4° C. for at least six months. In some embodiments, the mRNA maintains an integrity of 83% or greater after storage at 4° C. for at least six months. In some embodiments, the mRNA maintains an integrity of 84% or greater after storage at 4° C. for at least six months. In some embodiments, the mRNA maintains an integrity of 85% or greater after storage at 4° C. for at least six months. In some embodiments, the mRNA maintains an integrity of 86% or greater after storage at 4° C. for at least six months. In some embodiments, the mRNA maintains an integrity of 87% or greater after storage at 4° C. for at least six months. In some embodiments, the mRNA maintains an integrity of 88% or greater after storage at 4° C. for at least six months. In some embodiments, the mRNA maintains an integrity of 89% or greater after storage at 4° C. for at least six months. In some embodiments, the mRNA maintains an integrity of 90% or greater after storage at 4° C. for at least six months. In some embodiments, the mRNA maintains an integrity of 91% or greater after storage at 4° C. for at least six months. In some embodiments, the mRNA maintains an integrity of 92% or greater after storage at 4° C. for at least six months. In some embodiments, the mRNA maintains an integrity of 93% or greater after storage at 4° C. for at least six months. In some embodiments, the mRNA maintains an integrity of 94% or greater after storage at 4° C. for at least six months. In some embodiments, the mRNA maintains an integrity of 95% or greater after storage at 4° C. for at least six months. In some embodiments, the mRNA maintains an integrity of 96% or greater after storage at 4° C. for at least six months. In some embodiments, the mRNA maintains an integrity of 97% or greater after storage at 4° C. for at least six months. In some embodiments, the mRNA maintains an integrity of 98% or greater after storage at 4° C. for at least six months. In some embodiments, the mRNA maintains an integrity of 99% or greater after storage at 4° C. for at least six months.

In some embodiments, the mRNA maintains an integrity of 60% or greater, such as 65%, 70%, 75%, 80%, 85%, 90%, 92%, 94%, 96%, 98% or greater, including any values and subranges therebetween, after storage at, for example, 2-8° C. (e.g., 4° C.) for at least one year. In some embodiments, the mRNA maintains an integrity of 60% or greater, such as 65%, 70%, 75%, 80%, 85%, 90%, 92%, 94%, 96%, 98% or greater, including any values and subranges therebetween, after storage at, for example, 2-8° C. (e.g., 4° C.) for more than one year.

In some embodiments, the mRNA maintains an integrity of about 60%, 65%, 70%, 75%, 80%, 85%, 90%, 92%, 94%, 96%, 98% or greater, including any values and subranges therebetween, after storage at 25° C. for at least six months. In some embodiments, the mRNA maintains an integrity of 60% or greater after storage at 25° C. for at least six months. In some embodiments, the mRNA maintains an integrity of 61% or greater after storage at 25° C. for at least six months. In some embodiments, the mRNA maintains an integrity of 62% or greater after storage at 25° C. for at least six months. In some embodiments, the mRNA maintains an integrity of 63% or greater after storage at 25° C. for at least six months. In some embodiments, the mRNA maintains an integrity of 64% or greater after storage at 25° C. for at least six months. In some embodiments, the mRNA maintains an integrity of 65% or greater after storage at 25° C. for at least six months. In some embodiments, the mRNA maintains an integrity of 66% or greater after storage at 25° C. for at least six months. In some embodiments, the mRNA maintains an integrity of 67% or greater after storage at 25° C. for at least six months. In some embodiments, the mRNA maintains an integrity of 68% or greater after storage at 25° C. for at least six months. In some embodiments, the mRNA maintains an integrity of 69% or greater after storage at 25° C. for at least six months. In some embodiments, the mRNA maintains an integrity of 70% or greater after storage at 25° C. for at least six months. In some embodiments, the mRNA maintains an integrity of 71% or greater after storage at 25° C. for at least six months. In some embodiments, the mRNA maintains an integrity of 72% or greater after storage at 25° C. for at least six months. In some embodiments, the mRNA maintains an integrity of 73% or greater after storage at 25° C. for at least six months. In some embodiments, the mRNA maintains an integrity of 74% or greater after storage at 25° C. for at least six months. In some embodiments, the mRNA maintains an integrity of 75% or greater after storage at 25° C. for at least six months. In some embodiments, the mRNA maintains an integrity of 76% or greater after storage at 25° C. for at least six months. In some embodiments, the mRNA maintains an integrity of 77% or greater after storage at 25° C. for at least six months. In some embodiments, the mRNA maintains an integrity of 78% or greater after storage at 25° C. for at least six months. In some embodiments, the mRNA maintains an integrity of 79% or greater after storage at 25° C. for at least six months. In some embodiments, the mRNA maintains an integrity of 80% or greater after storage at 25° C. for at least six months. In some embodiments, the mRNA maintains an integrity of 81% or greater after storage at 25° C. for at least six months. In some embodiments, the mRNA maintains an integrity of 82% or greater after storage at 25° C. for at least six months. In some embodiments, the mRNA maintains an integrity of 83% or greater after storage at 25° C. for at least six months. In some embodiments, the mRNA maintains an integrity of 84% or greater after storage at 25° C. for at least six months. In some embodiments, the mRNA maintains an integrity of 85% or greater after storage at 25° C. for at least six months. In some embodiments, the mRNA maintains an integrity of 86% or greater after storage at 25° C. for at least six months. In some embodiments, the mRNA maintains an integrity of 87% or greater after storage at 25° C. for at least six months. In some embodiments, the mRNA maintains an integrity of 88% or greater after storage at 25° C. for at least six months. In some embodiments, the mRNA maintains an integrity of 89% or greater after storage at 25° C. for at least six months. In some embodiments, the mRNA maintains an integrity of 90% or greater after storage at 25° C. for at least six months. In some embodiments, the mRNA maintains an integrity of 91% or greater after storage at 25° C. for at least six months. In some embodiments, the mRNA maintains an integrity of 92% or greater after storage at 25° C. for at least six months. In some embodiments, the mRNA maintains an integrity of 93% or greater after storage at 25° C. for at least six months. In some embodiments, the mRNA maintains an integrity of 94% or greater after storage at 25° C. for at least six months. In some embodiments, the mRNA maintains an integrity of 95% or greater after storage at 25° C. for at least six months. In some embodiments, the mRNA maintains an integrity of 96% or greater after storage at 25° C. for at least six months. In some embodiments, the mRNA maintains an integrity of 97% or greater after storage at 25° C. for at least six months. In some embodiments, the mRNA maintains an integrity of 98% or greater after storage at 25° C. for at least six months. In some embodiments, the mRNA maintains an integrity of 99% or greater after storage at 25° C. for at least six months.

In some embodiments, the mRNA maintains an integrity of about 60%, 65%, 70%, 75%, 80%, 85%, 90%, 92%, 94%, 96%, 98% or greater, including any values and subranges therebetween, after storage at −20° C. for at least six months. In some embodiments, the mRNA maintains an integrity of 60% or greater after storage at −20° C. for at least six months. In some embodiments, the mRNA maintains an integrity of 61% or greater after storage at-20° C. for at least six months. In some embodiments, the mRNA maintains an integrity of 62% or greater after storage at −20° C. for at least six months. In some embodiments, the mRNA maintains an integrity of 63% or greater after storage at −20° C. for at least six months. In some embodiments, the mRNA maintains an integrity of 64% or greater after storage at-20° C. for at least six months. In some embodiments, the mRNA maintains an integrity of 65% or greater after storage at −20° C. for at least six months. In some embodiments, the mRNA maintains an integrity of 66% or greater after storage at −20° C. for at least six months. In some embodiments, the mRNA maintains an integrity of 67% or greater after storage at-20° C. for at least six months. In some embodiments, the mRNA maintains an integrity of 68% or greater after storage at −20° C. for at least six months. In some embodiments, the mRNA maintains an integrity of 69% or greater after storage at −20° C. for at least six months. In some embodiments, the mRNA maintains an integrity of 70% or greater after storage at-20° C. for at least six months. In some embodiments, the mRNA maintains an integrity of 71% or greater after storage at −20° C. for at least six months. In some embodiments, the mRNA maintains an integrity of 72% or greater after storage at −20° C. for at least six months. In some embodiments, the mRNA maintains an integrity of 73% or greater after storage at-20° C. for at least six months. In some embodiments, the mRNA maintains an integrity of 74% or greater after storage at −20° C. for at least six months. In some embodiments, the mRNA maintains an integrity of 75% or greater after storage at −20° C. for at least six months. In some embodiments, the mRNA maintains an integrity of 76% or greater after storage at-20° C. for at least six months. In some embodiments, the mRNA maintains an integrity of 77% or greater after storage at −20° C. for at least six months. In some embodiments, the mRNA maintains an integrity of 78% or greater after storage at −20° C. for at least six months. In some embodiments, the mRNA maintains an integrity of 79% or greater after storage at-20° C. for at least six months. In some embodiments, the mRNA maintains an integrity of 80% or greater after storage at −20° C. for at least six months. In some embodiments, the mRNA maintains an integrity of 81% or greater after storage at −20° C. for at least six months. In some embodiments, the mRNA maintains an integrity of 82% or greater after storage at-20° C. for at least six months. In some embodiments, the mRNA maintains an integrity of 83% or greater after storage at −20° C. for at least six months. In some embodiments, the mRNA maintains an integrity of 84% or greater after storage at −20° C. for at least six months. In some embodiments, the mRNA maintains an integrity of 85% or greater after storage at-20° C. for at least six months. In some embodiments, the mRNA maintains an integrity of 86% or greater after storage at −20° C. for at least six months. In some embodiments, the mRNA maintains an integrity of 87% or greater after storage at −20° C. for at least six months. In some embodiments, the mRNA maintains an integrity of 88% or greater after storage at-20° C. for at least six months. In some embodiments, the mRNA maintains an integrity of 89% or greater after storage at −20° C. for at least six months. In some embodiments, the mRNA maintains an integrity of 90% or greater after storage at −20° C. for at least six months. In some embodiments, the mRNA maintains an integrity of 91% or greater after storage at-20° C. for at least six months. In some embodiments, the mRNA maintains an integrity of 92% or greater after storage at −20° C. for at least six months. In some embodiments, the mRNA maintains an integrity of 93% or greater after storage at −20° C. for at least six months. In some embodiments, the mRNA maintains an integrity of 94% or greater after storage at-20° C. for at least six months. In some embodiments, the mRNA maintains an integrity of 95% or greater after storage at −20° C. for at least six months. In some embodiments, the mRNA maintains an integrity of 96% or greater after storage at −20° C. for at least six months. In some embodiments, the mRNA maintains an integrity of 97% or greater after storage at-20° C. for at least six months. In some embodiments, the mRNA maintains an integrity of 98% or greater after storage at −20° C. for at least six months. In some embodiments, the mRNA maintains an integrity of 99% or greater after storage at −20° C. for at least six months.

In some embodiments, the mRNA maintains an integrity of 60% or greater, such as 65%, 70%, 75%, 80%, 85%, 90%, 92%, 94%, 96%, 98% or greater, including any values and subranges therebetween, after storage at −20° C. for at least one year.

g. Delivery Routes

In some embodiments, the reconstituted dry powder product comprising sugar or sugar alcohol enables administration of the mRNA-nanoparticle as a liquid formulation at a desirable dosage. As disclosed herein, the term “desirable dosage” is a dose that is suitable for the reconstitutable dry powder formulation described in the present disclosure. The pharmaceutical compositions comprising the reconstituted dry powder product as described herein may be administered to a subject in need thereof subconjunctival, sublingual by any one of several routes that effectively delivers an effective amount of the compound. Non-limiting examples of suitable administrative routes include topical, oral, nasal, intrathecal, enteral, buccal, sublingual, transdermal, rectal, vaginal, intraocular, and parenteral administration, i including subcutaneous, intravenous, intramuscular, intrasternal, intracavemous, intrameatal, and intraurethral injection and/or infusion.

In some embodiments, the reconstituted dry powder product is suitable for mucosal delivery. In some embodiments, the reconstituted dry powder product is suitable for oral delivery. In some embodiments, the reconstituted dry powder product is suitable for sublingual delivery. In some embodiments, the formulation is suitable for or intranasal delivery. In some embodiments, the reconstituted dry powder product is suitable for buccal delivery. In some embodiments, the reconstituted dry powder product is suitable for any delivery route that is suitable for the present disclosure.

### N/P Ratios of Lipid Nanoparticles

In some embodiments, the preparation of LNPs entails encapsulating mRNA lipids are added to the aqueous buffer containing mRNA at a certain Nitrogen (lipid) to Phosphate (nucleic acid) ratio (N/P ratio).

In some embodiments, mRNA and lipids are combined with pump systems which maintain the lipid/mRNA (N/P) ratio constant throughout the process and which can also afford facile scale-up.

In some embodiments, the one or more LNPs encapsulating mRNA (also referred to as mRNA-loaded LNPs or mRNA-LNPs) have a lipid: mRNA (N/P) ratio ranging from 1 to 20, 1 to 15, 1 to 10, 2 to 8, 2 to 6, or 2 to 4. In some embodiments, the one or more mRNA-loaded lipid nanoparticles have a lipid: mRNA (N/P) ratio ranging from 1 to 20. In some embodiments, the one or more mRNA-loaded lipid nanoparticles have a lipid: mRNA (N/P) ratio ranging from 1 to 18. In some embodiments, the one or more mRNA-loaded lipid nanoparticles have a lipid: mRNA (N/P) ratio ranging from 1 to 16. In some embodiments, the one or more mRNA-loaded lipid nanoparticles have a lipid: mRNA (N/P) ratio ranging from 1 to 14. In some embodiments, the one or more mRNA-loaded lipid nanoparticles have a lipid: mRNA (N/P) ratio ranging from 1 to 12. In some embodiments, the one or more mRNA-loaded lipid nanoparticles have a lipid: mRNA (N/P) ratio ranging from 1 to 10. In some embodiments, the one or more mRNA-loaded lipid nanoparticles have a lipid: mRNA (N/P) ratio ranging from 1 to 8. In some embodiments, the one or more mRNA-loaded lipid nanoparticles have a lipid: mRNA (N/P) ratio ranging from 1 to 6. In some embodiments, the one or more mRNA-loaded lipid nanoparticles have a lipid: mRNA (N/P) ratio ranging from 2 to 20. In some embodiments, the one or more mRNA-loaded lipid nanoparticles have a lipid: mRNA (N/P) ratio ranging from 2 to 16. In some embodiments, the one or more mRNA-loaded lipid nanoparticles have a lipid: mRNA (N/P) ratio ranging from 2 to 12. In some embodiments, the one or more mRNA-loaded lipid nanoparticles have a lipid: mRNA (N/P) ratio ranging from 2 to 8. In some embodiments, the one or more mRNA-loaded lipid nanoparticles have a lipid: mRNA (N/P) ratio ranging from 2 to 6. In some embodiments, the one or more mRNA-loaded lipid nanoparticles have a lipid: mRNA (N/P) ratio ranging from 2 to 4. In some embodiments, the one or more mRNA-loaded lipid nanoparticles have a lipid: mRNA (N/P) ratio ranging from 4 to 20. In some embodiments, the one or more mRNA-loaded lipid nanoparticles have a lipid: mRNA (N/P) ratio ranging from 4 to 16. In some embodiments, the one or more mRNA-loaded lipid nanoparticles have a lipid: mRNA (N/P) ratio ranging from 4 to 14. In some embodiments, the one or more mRNA-loaded lipid nanoparticles have a lipid: mRNA (N/P) ratio ranging from 4 to 12. In some embodiments, the one or more mRNA-loaded lipid nanoparticles have a lipid: mRNA (N/P) ratio ranging from 4 to 10. In some embodiments, the one or more mRNA-loaded LNPs have a lipid: mRNA (N/P) ratio of 2 or 4. In some embodiments, the one or more mRNA-loaded LNPs have a lipid: mRNA (N/P) ratio of 2. In some embodiments, the one or more mRNA-loaded LNPs have a lipid: mRNA (N/P) ratio of 4.

In some embodiments, the lipid nanoparticles encapsulating the mRNA have an N/P ratio of between 2 and 6. In some embodiments, the N/P ratio is between 3 and 4. In some embodiments, the N/P ratio is 3.

In some embodiments, a dry powder formulation comprises lipid nanoparticles that are added to the mRNA at an N:P ratio of about 1. In some embodiments, a dry powder formulation comprises lipid nanoparticles are added to the mRNA at an N:P ratio of about 2. In some embodiments, a dry powder formulation comprises lipid nanoparticles are added to the mRNA at an N:P ratio of about 3. In some embodiments, a dry powder formulation comprises lipid nanoparticles are added to the mRNA at an N:P ratio of about 4. In some embodiments, a dry powder formulation comprises lipid nanoparticles are added to the mRNA at an N:P ratio of about 5. In some embodiments, a dry powder formulation comprises lipid nanoparticles are added to the mRNA at an N:P ratio of about 6. In some embodiments, a dry powder formulation comprises lipid nanoparticles are added to the mRNA at an N:P ratio of about 7. In some embodiments, a dry powder formulation comprises lipid nanoparticles are added to the mRNA at an N:P ratio of about 8.

### Encapsulation Efficiencies

The dry powder formulations described herein can have unexpectedly improved encapsulation efficiencies, including according to the further exemplary embodiments provided below.

In some embodiments, the one or more mRNA-loaded lipid nanoparticles of the dry powder formulations described herein have an encapsulation efficiency of about 70%, 75%, 80%, 85%, 90%, 92%, 94%, 96%, 98% or greater, including any values and subranges therebetween. In some embodiments, the one or more mRNA-loaded lipid nanoparticles have an encapsulation efficiency of 70% or greater. In some embodiments, the one or more mRNA-loaded lipid nanoparticles have an encapsulation efficiency of 71% or greater. In some embodiments, the one or more mRNA-loaded lipid nanoparticles have an encapsulation efficiency of 72% or greater. In some embodiments, the one or more mRNA-loaded lipid nanoparticles have an encapsulation efficiency of 73% or greater. In some embodiments, the one or more mRNA-loaded lipid nanoparticles have an encapsulation efficiency of 74% or greater. In some embodiments, the one or more mRNA-loaded lipid nanoparticles have an encapsulation efficiency of 75% or greater. In some embodiments, the one or more mRNA-loaded lipid nanoparticles have an encapsulation efficiency of 76% or greater. In some embodiments, the one or more mRNA-loaded lipid nanoparticles have an encapsulation efficiency of 77% or greater. In some embodiments, the one or more mRNA-loaded lipid nanoparticles have an encapsulation efficiency of 78% or greater. In some embodiments, the one or more mRNA-loaded lipid nanoparticles have an encapsulation efficiency of 79% or greater. In some embodiments, the one or more mRNA-loaded lipid nanoparticles have an encapsulation efficiency of 80% or greater. In some embodiments, the one or more mRNA-loaded lipid nanoparticles have an encapsulation efficiency of 81% or greater. In some embodiments, the one or more mRNA-loaded lipid nanoparticles have an encapsulation efficiency of 82% or greater. In some embodiments, the one or more mRNA-loaded lipid nanoparticles have an encapsulation efficiency of 83% or greater. In some embodiments, the one or more mRNA-loaded lipid nanoparticles have an encapsulation efficiency of 84% or greater. In some embodiments, the one or more mRNA-loaded lipid nanoparticles have an encapsulation efficiency of 85% or greater. In some embodiments, the one or more mRNA-loaded lipid nanoparticles have an encapsulation efficiency of 86% or greater. In some embodiments, the one or more mRNA-loaded lipid nanoparticles have an encapsulation efficiency of 87% or greater. In some embodiments, the one or more mRNA-loaded lipid nanoparticles have an encapsulation efficiency of 88% or greater. In some embodiments, the one or more mRNA-loaded lipid nanoparticles have an encapsulation efficiency of 89% or greater. In some embodiments, the one or more mRNA-loaded lipid nanoparticles have an encapsulation efficiency of 90% or greater. In some embodiments, the one or more mRNA-loaded lipid nanoparticles have an encapsulation efficiency of 91% or greater. In some embodiments, the one or more mRNA-loaded lipid nanoparticles have an encapsulation efficiency of 92% or greater. In some embodiments, the one or more mRNA-loaded lipid nanoparticles have an encapsulation efficiency of 93% or greater. In some embodiments, the one or more mRNA-loaded lipid nanoparticles have an encapsulation efficiency of 94% or greater. In some embodiments, the one or more mRNA-loaded lipid nanoparticles have an encapsulation efficiency of 95% or greater. In some embodiments, the one or more mRNA-loaded lipid nanoparticles have an encapsulation efficiency of 96% or greater. In some embodiments, the one or more mRNA-loaded lipid nanoparticles have an encapsulation efficiency of 97% or greater. In some embodiments, the one or more mRNA-loaded lipid nanoparticles have an encapsulation efficiency of 98% or greater. In some embodiments, the one or more mRNA-loaded lipid nanoparticles have an encapsulation efficiency of 99% or greater.

In some embodiments, the reconstituted dry powder formulation described herein provides comparable encapsulation efficiencies as compared to an equivalent non-dry powder formulation. In some embodiments, the encapsulation efficiency of the one or more mRNA-loaded lipid nanoparticles of the dry powder formulations described herein does not appreciably change during storage of the reconstituted dry powder formulation, including any exemplary storage condition, duration, and/or temperature described herein. In some embodiments, the encapsulation efficiency of the one or more mRNA-loaded lipid nanoparticles of the reconstituted dry powder formulation does not change by more than about 1%, 2%, 2.5%, 4%, 5%, 7.5%, 10%, 12.5%, 15%, 18.5%, 20%, 25%, 30%, 35%, 40%, 50%, including all values and ranges therebetween, (e.g., no more than about 30%, 25%, 20%, 15%, 10%, or 5%, including all values and ranges therebetween) following storage, including according to any exemplary storage condition, duration, and/or temperature as described herein.

In some embodiments of the reconstituted dry powder formulation described herein provides comparable encapsulation efficiencies as compared to the reconstituted dry powder formulation prior to reconstitution. In some embodiments, the encapsulation efficiency of the one or more mRNA-loaded lipid nanoparticles of the reconstituted dry powder formulation described herein does not change by more than about 1%, 2%, 2.5%, 4%, 5%, 7.5%, 10%, 12.5%, 15%, 18.5%, 20%, 25%, 30%, 35%, 40%, 50%, including all values and ranges therebetween, (e.g., no more than about 30%, 25%, 20%, 15%, 10%, or 5%, including all values and ranges therebetween) following reconstitution, including according to any method as described herein.

### Pre-Spray Drying LNP Sizes and Sizing

As described herein, dry powder formulations described herein can be formulated to provide the optimal lipid nanoparticle size for various uses, including according to the further exemplary embodiments provided below.

Suitable mRNA loaded lipid nanoparticles may be made in various sizes.

In some embodiments, the size of an mRNA loaded lipid nanoparticles pre-spray drying is determined by the length of the largest diameter of the lipid nanoparticle. In some embodiments, an mRNA loaded lipid nanoparticle has a size pre-spray drying no greater than about 250 nm (e.g., no greater than about 250 nm, about 225 nm, about 200 nm, about 175 nm, about 150 nm, about 125 nm, about 100 nm, about 90 nm, about 80 nm, about 75 nm, about 70 nm, about 60 nm, about 50 nm, about 40 nm, about 30 nm, about 25 nm, about 20 nm, or about 10 nm, including all values and ranges therebetween). In some embodiments, a suitable liposome has a size ranging from about 10 nm to about 250 nm (e.g., ranging from about 10 nm to about 225 nm, about 10 nm to about 200 nm, about 10 nm to about 175 nm, about 10 nm to about 150 nm, about 10 nm to about 125 nm, about 10 nm to about 100 nm, about 10 nm to about 75 nm, or about 10 nm to about 50 nm). In some embodiments, an mRNA loaded lipid nanoparticle has a size pre-spray drying ranging from about 100 nm to about 250 nm (e.g., ranging from about 100 nm to about 225 nm, about 100 nm to about 200 nm, about 100 nm to about 175 nm, about 100 nm to about 150 nm). In some embodiments, an mRNA loaded lipid nanoparticle has a size pre-spray drying ranging from about 10 nm to about 100 nm (e.g., ranging from about 10 nm to about 90 nm, about 10 nm to about 80 nm, about 10 nm to about 70 nm, about 10 nm to about 60 nm, or about 10 nm to about 50 nm). In a particular embodiment, an mRNA loaded lipid nanoparticle has a size pre-spray drying less than about 100 nm.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-150 (1981), incorporated herein by reference. Average liposome diameter may be reduced by sonication of formed liposomes. Intermittent sonication cycles may be alternated with QELS assessment to guide efficient liposome synthesis.

### Spray-Drying Process

Spray drying is a commonly used, economical, and established technique to manufacture the dry powder product for various modalities such as small molecules, peptides, and proteins. The technique is continuous, scalable, suitable for heat sensitive material, can produce consistent dry powder product, and can be automated. Various sugars such as lactose and mannitol are commonly used as carrier excipients to facilitate the spray drying process. For example, in the compositions and processes described herein, beneficial properties of mannitol as an excipient for spray drying of mRNA formulations include (i) altering effect on the viscoelastic properties associated to the phlegm (ii) increasing water content driven by osmotic gradient (iii) less hygroscopic compared to some other sugars like lactose (iv) non-reducing sugar with absence of aldehyde group etc. However, amino acids such as leucine, isoleucine, and trileucine have been utilized to increase the dispersibility and decrease the MMAD of dry powder product. Although spray drying is continuous, scalable, suitable for heat sensitive material and can produce consistent DPP, reports of excipient screening and optimization of formulation properties and aerosol performance of spray dried mRNA LNPs are minimal.

Various spray-drying processes may be used to practice the present disclosure.

The process involves, in general, removal of moisture from a composition by passing a liquid form of the composition through an apparatus. For example, a liquid formulation comprising the composition of interest is passed through a narrow inlet spray ‘atomizer’ nozzle into a first chamber, which is the drying chamber. Usually, the liquid formulation is passed in a steady stream. The liquid formulation is sprayed as tiny droplets into the drying chamber. A stream of heated air or gas is also led into the drying chamber to form an air current. Passage of the formulation through this heated current disperses the incoming droplets, dries them into the solid particulate form. This product is led into a second chamber by flow through a connector or pipe. The second chamber is the cyclone powder collector. Here, the air circulation generates a cyclone, and the powder particles are collected via a vortex stream into a collection vessel attached to the outlet end. The cyclone chamber is attached to an exhaust fan, which helps cool the components. The inlet and outlet temperatures are operator adjustable. The respective inlet and outlet temperatures, the chamber temperatures, the liquid feed flow rate (aspirator %), pressure, nature of the heated air current and most importantly, the composition of the liquid feed are suitably adjusted for optimal drying of any particulate matter.

In some embodiments, the inlet temperature is adjustable within a range of 40° C. to 200° C. In some embodiments, the outlet temperature ranges between 20-70° C. The relative pressure of the pump and the aspirator is also operator adjustable. In some embodiments, the inlet temperature was adjusted to 55° C. In some embodiments, the inlet temperature was adjusted to 60° C. In some embodiments, the inter temperature was adjusted to 61° C. In some embodiments, the inter temperature was adjusted to 62° C. In some embodiments, the inter temperature was adjusted to 63° C. In some embodiments, the inter temperature was adjusted to 64° C. In some embodiments, the inter temperature was adjusted to 65° C. In some embodiments, the inter temperature was adjusted to 66° C. In some embodiments, the inter temperature was adjusted to 67° C. In some embodiments, the inter temperature was adjusted to 68° C. In some embodiments, the inter temperature was adjusted to 69° C. In some embodiments, the inter temperature was adjusted to 70° C.

In some embodiments, for spray-drying mRNA-lipid nanoparticle, the inlet temperature is adjusted between 70° C. and 200° C. In some embodiments, the inlet temperature is adjusted between 80° C. and 200° C. In some embodiments, the inlet temperature is adjusted between 90° C. and 200° C. In some embodiments, the inlet temperature is adjusted between 95° C. and 180° C. In some embodiments, the inlet temperature is adjusted between 95° C. and 160° C. In some embodiments, the inlet temperature is adjusted between 90° C. and 150° C. In some embodiments, the inlet temperature is adjusted between 90° C. and 120° C. In some embodiments the inlet temperature is adjusted between 90° C. and 100° C. In some embodiments, the inlet temperature is 70° C., 75° C., 80° C., 85° C., 90° C., 95° C., or 100° C., including any values and subranges therebetween.

In some embodiments, the outlet temperature ranges between 20° C. to 70° C. In some embodiments, the outlet temperature is between 30° C. to 60° C. In some embodiments, the outlet temperature is between 20° C. to 50° C. In some embodiments, the outlet temperature is between 30° C. to 50° C. In some embodiments, the outlet temperature is between 40° C. to 50° C. In some embodiments, the outlet temperature is between 45° C. and 50° C.

Spray drying can be carried out using any suitable spray-drying device. As is known to a person of ordinary skill in the art, a variety of spray-drying instruments are commercially available and can be used to practice the present disclosure. Exemplary commercially available devices suitable for the present disclosure include, but are not limited to the following: Mini Spray Dryer B-290; Nano Spray Dryer B-90 (manufactured by Buchi); Anhydro MicraSpray Dryer GMP; Anhydro MicraSpray Dryer Aseptic series (manufactured by SPX FLOW); MDL-50 and MDL-015 (manufactured by Fujisaki Electric); Versatile Mini Sprayer Dryer GAS410 (manufactured by Yamato Scientific America); LSD-1500 Mini spray dryer, MSD-8 Multi-functional laboratory spray dryer; PSD-12 Precision pharmacy spray dryer; (manufactured by Changzhou Xiandao Drying Equipment Co. Ltd); TALL FORM DRYER™; Multi-Stage Dryer; COMPACT DRYER™; FILTERMAT™ Spray Dryer; VERSATILE-SD™; Fluidized Spray Dryer; MOBILE MINOR™; SDMICRO™; PRODUCTION MINOR™ (manufactured by GEA Process Engineering) and many others. Convenient scale up from laboratory scale to industrial manufacturing scale is also available from several of these manufacturers.

### Dry Powders

Dry powders prepared according to the present disclosure contain a plurality of spray-dried particles. Residual moisture content, aerosol performance and physio-chemical stability are important parameters for spray-dried pharmaceutical products. It is determined by the sample weight loss after heating and drying, using the equation:

$$Moisture content \% = \frac{( {SW}_{b} - {SW}_{a} )}{{SW}_{b}} \times 100 \% ,$$

wherein, SW_{b} is the sample weight before heating and SWₐ is the sample weight after heating. Perkin Elmer TGA 7 (Perkin Elmer) is an example of commercially used instrument with associated software for the measurement of residual moisture in a nanoparticle.

In general, an admissible range of particle size distribution is maintained for uniformity of dosing of an active pharmaceutical ingredient of the formulation. In some embodiments, the particles of the dry powder formulation suitable for pulmonary administration comprise trehalose. In some embodiments, the particles of the dry powder formulation suitable for pulmonary administration, comprising trehalose are 5 micrometer or less. In some embodiments, spraying lipid nanoparticles using hydroalcoholic solution aids in reducing the particle size of the DPP. In some embodiments, spraying lipid nanoparticles using hydroalcoholic solution comprising sugar or sugar alcohol aids in reducing particle of DPP. In some embodiments, the sugar is mannitol, xylitol, lactose, sucrose, or trehalose. In some embodiments, the sugar is trehalose. In some embodiments, the sugar or sugar alcohol is mannitol. In some embodiments, the sugar is sucrose. In some embodiments, the sugar or sugar alcohol is xylitol. In some embodiments, the sugar is lactose.

In some embodiments, the particle size (e.g., mean particle size) of the dry powder is between 0.5-10 μm. In some embodiments, the particle size (e.g., mean particle size) of the dry powder formulation is between 1-8 μm. In some embodiments, the particle size (e.g., mean particle size) of the dry powder formulation is between 1-7 μm. In some embodiments, the particle size (e.g., mean particle size) of the dry powder formulation is between 1-6 μm. In some embodiments, the particle size (e.g., mean particle size) of the dry powder is between 1-5 μm. In some embodiments, the particle size (e.g., mean particle size) of the dry powder is between 1-4 μm. In some embodiments, the particle size (e.g., mean particle size) of the dry powder is between 1-3 μm. In some embodiments, the particle size (e.g., mean particle size) of the dry powder is between 1-2 μm. In some embodiments, the particle size (e.g., mean particle size) of the dry powder is about lum. In some embodiments, the particle size (e.g., mean particle size) of the dry powder is about 2 μm. In some embodiments, the particle size (e.g., mean particle size) of the dry powder is about 3 μm. In some embodiments, the particle size (e.g., mean particle size) of the dry powder is about 4 μm. In some embodiments, the particle size (e.g., mean particle size) of the dry powder is about 5 μm. In some embodiments, the particle size (e.g., mean particle size) of the dry powder is about 6 μm. In some embodiments, the particle size (e.g., mean particle size) of the dry powder is about 7 μm. In some embodiments, the particle size (e.g., mean particle size) of the dry powder is about 8 μm. In some embodiments, the particle size (e.g., mean particle size) of the dry powder is about 9 μm. In some embodiments, the particle size (e.g., mean particle size) of the dry powder is about 10 μm.

Primary particle size (e.g., mean particle size) distributions of spray-dried particles are measured by dynamic light scattering, which is expressed in terms of Z-average. The Z-average is the mean, also known as the cumulant size, calculated from the intensity-weighted distribution of particle diameter and is given by the formula,

$${D}_{Z} = \frac{\sum {S}_{i}}{\sum [ {S}_{i} / {D}_{i} ]}$$

where, Sᵢ is the scattered intensity from the particle ‘i’, and Di is the particle's diameter. In addition to these parameters, a fine and a course fraction of the particles is defined.

Polydispersity index (PDI), on the other hand is the measure of the distribution of molecular mass of a given particulate sample. In some embodiments, the polydispersity index of the glycerol and propylene glycol based LNP is less than 0.2. In some embodiments, the polydispersity index of the glycerol and propylene glycol based LNP is about 0.1. In some embodiments, the polydispersity index of the glycerol and propylene glycol based LNP is less than about 0.1.

mRNA

In some embodiments, the mRNA constitutes greater than about 2% of the dry powder formulation by weight. In some embodiments, the mRNA constitutes greater than about 3% of the dry powder formulation by weight. In some embodiments, the mRNA constitutes greater than about 4% of the dry powder formulation by weight.

It was observed that the dry powder formulation of mRNA of the present disclosure had high stability. In some embodiments, the mRNA in the dry powder formulation maintains greater than about 80% integrity. In some embodiments, the mRNA in the dry powder formulation maintains greater than about 81% integrity. In some embodiments, the mRNA in the dry powder formulation maintains greater than about 82% integrity. In some embodiments, the mRNA in the dry powder formulation maintains greater than about 83% integrity. In some embodiments, the mRNA in the dry powder formulation maintains greater than about 84% integrity. In some embodiments, the mRNA in the dry powder formulation maintains greater than about 85% integrity. In some embodiments, the mRNA in the dry powder formulation maintains greater than about 86% integrity. In some embodiments, the mRNA in the dry powder formulation maintains greater than about 87% integrity. In some embodiments, the mRNA in the dry powder formulation maintains greater than about 88% integrity. In some embodiments, the mRNA in the dry powder formulation maintains greater than about 89% integrity. In some embodiments, the mRNA in the dry powder formulation maintains greater than about 90% integrity. In some embodiments, the mRNA in the dry powder formulation maintains greater than about 91% integrity. In some embodiments, the mRNA in the dry powder formulation maintains greater than about 92% integrity. In some embodiments, the mRNA in the dry powder formulation maintains greater than about 93% integrity. In some embodiments, the mRNA in the dry powder formulation maintains greater than about 94% integrity. In some embodiments, the mRNA in the dry powder formulation maintains greater than about 95% integrity. In some embodiments, the mRNA in the dry powder formulation maintains greater than about 96% integrity. In some embodiments, the mRNA in the dry powder formulation maintains greater than about 97% integrity. In some embodiments, the mRNA in the dry powder formulation maintains greater than about 98% integrity. In some embodiments, the mRNA in the dry powder formulation maintains greater than about 99% integrity.

It is desired that the integrity of the mRNA be maintained after multiple freeze-thaw cycles and/or for prolonged periods of time for maintaining therapeutic benefit. In some embodiments, the mRNA maintains integrity of 80% or greater after storage at room temperature for 6 months or longer. In some embodiments, the mRNA maintains integrity of 80% or greater after storage at room temperature for 3 months or longer. In some embodiments, the mRNA maintains integrity of 80% or greater after storage at room temperature for 1 year or longer. In some embodiments, the mRNA maintains integrity of 90% or greater after storage at room temperature for 6 months or longer. In some embodiments, the mRNA maintains integrity of 90% or greater after storage at room temperature for 3 months or longer. In some embodiments, the mRNA maintains integrity of 90% or greater after storage at room temperature for 1 year or longer.

In some embodiments, the mRNA maintains integrity of 80%, 85%, 90%, 92%, 94%, 96%, 98% or greater, including any values and subranges therebetween, after storage at 2-8° C. (e.g., 4° C.) for 4 weeks or longer, such as 1 months, 3 months, 6 months, 9 months, or 1 year or longer, including any values and subranges therebetween. In some embodiments, the mRNA maintains integrity of 80% or greater after storage at 2-8° C. (e.g., 4° C.) for 4 weeks or longer. In some embodiments, the mRNA maintains integrity of 80% or greater after storage at 2-8° C. (e.g., 4° C.) for 3 months or longer. In some embodiments, the mRNA maintains integrity of 80% or greater after storage at 2-8° C. (e.g., 4° C.) for 6 months or longer. In some embodiments, the mRNA maintains integrity of 80% or greater after storage at 2-8° C. (e.g., 4° C.) for a year or longer. In some embodiments, the mRNA maintains integrity of 85% or greater after storage at 2-8° C. (e.g., 4° C.) for 4 weeks or longer. In some embodiments, the mRNA maintains integrity of 85% or greater after storage at 2-8° C. (e.g., 4° C.) for 3 months or longer. In some embodiments, the mRNA maintains integrity of 85% or greater after storage at 2-8° C. (e.g., 4° C.) for 6 months or longer. In some embodiments, the mRNA maintains integrity of 85% or greater after storage at 2-8° C. (e.g., 4° C.) for a year or longer. In some embodiments, the mRNA maintains integrity of 90% or greater after storage at 2-8° C. (e.g., 4° C.) for 4 weeks or longer. In some embodiments, the mRNA maintains integrity of 90% or greater after storage at 2-8° C. (e.g., 4° C.) for 3 months or longer. In some embodiments, the mRNA maintains integrity of 90% or greater after storage at 2-8° C. (e.g., 4° C.) for 6 months or longer. In some embodiments, the mRNA maintains integrity of 90% or greater after storage at 2-8° C. (e.g., 4° C.) for a year or longer. In some embodiments, the mRNA maintains integrity of 95% or greater after storage at 2-8° C. (e.g., 4° C.) for 4 weeks or longer. In some embodiments, the mRNA maintains integrity of 95% or greater after storage at 2-8° C. (e.g., 4° C.) for 3 months or longer. In some embodiments, the mRNA maintains integrity of 95% or greater after storage at 2-8° C. (e.g., 4° C.) for 6 months or longer. In some embodiments, the mRNA maintains integrity of 95% or greater after storage at 2-8° C. (e.g., 4° C.) for a year or longer.

In some embodiments, the mRNA maintains integrity of 80% or greater after storage at −80° C. after 3 freeze thaw cycles. In some embodiments, the mRNA maintains integrity of 90% or greater after storage at −80° C. after 3 freeze thaw cycles.

As used herein, the phrase “the mRNA maintains integrity of x % or greater after storage” means that the mRNA integrity does not decrease more than (100-x) % after storage.

mRNA Synthesis

mRNAs according to the present disclosure may be synthesized according to any of a variety of known methods. Various methods are described in published U.S. Application No. US 2018/0258423, and can be used to practice the present disclosure, all of which are incorporated herein by reference. For example, mRNAs according to the present disclosure may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

In some embodiments, a suitable mRNA sequence is an mRNA sequence encoding a protein or a peptide. In some embodiments, a suitable mRNA sequence is codon optimized for efficient expression human cells. In some embodiments, a suitable mRNA sequence is naturally-occurring or a wild-type sequence. In some embodiments, a suitable mRNA sequence encodes a protein or a peptide that contains one or mutations in amino acid sequence. An exemplary mRNA coding sequence and the corresponding amino acid sequence are shown below:

Exemplary Construct Design for mRNAs

X-mRNA coding region-Y
5′ and 3′ UTR Sequences
X (5′ UTR Sequence) =
(SEQ ID NO: 1)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG
Y (3′ UTR Sequence) =
(SEQ ID NO: 2)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UCAAGCU
OR
(SEQ ID NO: 3)
GGGUGGCAUCCCUGUGACCCCUCCCCAGUGCCUCUCCUGGCCCUGGAAG
UUGCCACUCCAGUGCCCACCAGCCUUGUCCUAAUAAAAUUAAGUUGCAU
CAAAGCU

The present disclosure may be used to deliver mRNAs of a variety of lengths. In some embodiments, the present disclosure may be used to deliver in vitro synthesized mRNA of or greater than about 0.5 kb, 1 kb, 1.5 kb, 2 kb, 2.5 kb, 3 kb, 3.5 kb, 4 kb, 4.5 kb, 5 kb, 6 kb, 7 kb, 8 kb, 9 kb, 10 kb, 11 kb, 12 kb, 13 kb, 14 kb, 15 kb, 20 kb, 30 kb, 40 kb, or 50 kb in length, including any values and subranges therebetween. In some embodiments, the present disclosure may be used to deliver in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-50 kb in length.

In some embodiments, for the preparation of mRNA according to the disclosure, a DNA template is transcribed in vitro. A suitable DNA template typically has a promoter, for example a T3, T7 or SP6 promoter, for in vitro transcription, followed by desired nucleotide sequence for desired mRNA and a termination signal.

### Nucleotides

Various naturally-occurring or modified nucleosides may be used to produce mRNA according to the present disclosure. In some embodiments, an mRNA is or comprises naturally-occurring nucleosides (or unmodified nucleotides; e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, a suitable mRNA may contain backbone modifications, sugar modifications and/or base modifications. For example, modified nucleotides may include, but not be limited to, modified purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, the mRNA comprises one or more nonstandard nucleotide residues. The nonstandard nucleotide residues may include, e.g., 5-methyl-cytidine (“5mC”), pseudouridine (“yU”), and/or 2-thio-uridine (“2sU”). See, e.g., U.S. Pat. No. 8,278,036 or WO 2011/012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US 2012/0195936 and international publication WO 2011/012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100%, including any values and subranges therebetween, of the constituent nucleotides individually or in combination.

In some embodiments, mRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g., cytidine 5′-O-(1-thiophosphate)), boranophosphates, positively charged guanidinium groups etc., which means by replacing the phosphodiester linkage by other anionic, cationic or neutral groups.

In some embodiments, mRNAs may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

### Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′ (A,G (5′)ppp(5′) A and G(5′)ppp(5′)G. Additional cap structures are described in published U.S. Application No. US 2016/0032356 and published U.S. Application No. US 2018/0125989, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively, including any values and subranges therebetween. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly(A) and poly(C) tails with various lengths described herein. In some embodiments, a tail structure includes at least 50%, 55%, 60%, 65%, 70%, 75%, 80%, 85%, 90%, 91%, 92%, 93%, 94%, 95%, 96%, 97%, 98%, or 99% adenosine nucleotides, including any values and subranges therebetween. In some embodiments, a tail structure includes at least 50%, 55%, 60%, 65%, 70%, 75%, 80%, 85%, 90%, 91%, 92%, 93%, 94%, 95%, 96%, 97%, 98%, or 99% cytosine nucleotides, including any values and subranges therebetween.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized according to the present disclosure may be used without further purification. In particular, mRNA synthesized according to the present disclosure may be used without a step of removing shortmers. In some embodiments, mRNA synthesized according to the present disclosure may be further purified. Various methods may be used to purify mRNA synthesized according to the present disclosure. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art. In some embodiments, the mRNA is purified using Tangential Flow Filtration. Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present disclosure.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation. In some embodiments, the mRNA is purified either before or after or both before and after capping and tailing, by filtration. In some embodiments, the mRNA is purified either before or after or both before and after capping and tailing, by Tangential Flow Filtration (TFF). In some embodiments, the mRNA is purified either before or after or both before and after capping and tailing by chromatography.

### Additional Lipids

In some embodiments, lipid nanoparticles comprise one or more cationic lipids, one or more non-cationic lipids, and one or more PEG-modified lipids. In some embodiments, lipid nanoparticles further comprise one or more cholesterol-based lipids. In some embodiments, the one or more cationic lipids constitutes about 30-70% of the total lipids in LNPs by molar %. In some embodiments, the one or more PEG-modified lipids constitutes about 1-15% of the total lipids in LNP by molar %. In some embodiments, the one or more non-cationic lipids constitutes about 10-40% of the total lipids in LNP by molar %. In some embodiments, the one or more cholesterol-based lipids comprise about 5-40% of the total lipids in LNP by molar %.

In some embodiments, the molar ratio of cationic lipid(s) to non-cationic lipid(s) to cholesterol-based lipid(s) to PEG-modified lipid(s) in the lipid nanoparticles is approximately 60:25:10:5. In some embodiments, the molar ratio of cationic lipid(s) to non-cationic lipid(s) to cholesterol-based lipid(s) to PEG-modified lipid(s) in the lipid nanoparticles is approximately 40:25:30:5.

Exemplary lipids are described herein.

### Cationic Lipids

As used herein, the phrase “cationic lipids” refers to any of a number of lipid species that have a net positive charge at a selected pH, such as physiological pH.

Suitable cationic lipids for use in the compositions and methods of the disclosure include the cationic lipids as described in International Patent Publication WO 2010/144740, which is incorporated herein by reference. In some embodiments, the compositions and methods of the present disclosure include a cationic lipid, (6Z,9Z,28Z,31Z)-heptatriaconta-6,9,28,31-tetraen-19-yl 4-(dimethylamino)butanoate, having a compound structure of:

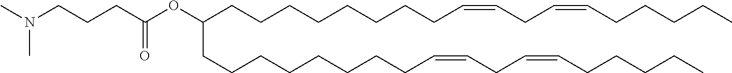 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present disclosure include ionizable cationic lipids as described in International Patent Publication WO 2013/149140, which is incorporated herein by reference. In some embodiments, the compositions and methods of the present disclosure include a cationic lipid of one of the following formulas:

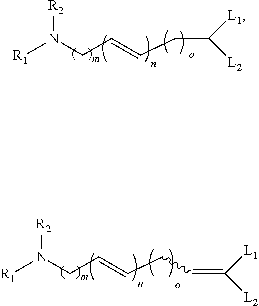

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In some embodiments, the compositions and methods of the present disclosure include the cationic lipid (15Z, 18Z)-N,N-dimethyl-6-(9Z,12Z)-octadeca-9,12-dien-1-yl) tetracosa-15,18-dien-1-amine (“HGT5000”), having a compound structure of:

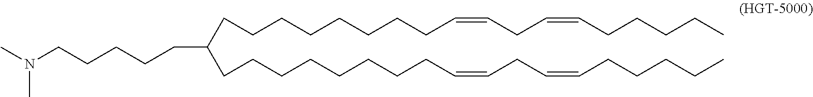 [SMILES: CCCCC/C=C\C/C=C\CCCCCCCCC(CCCCCCCC/C=C\C/C=C\CCCCC)CCCCCN(C)C]

and pharmaceutically acceptable salts thereof. In some embodiments, the compositions and methods of the present disclosure include the cationic lipid (15Z, 18Z)-N,N-dimethyl-6-((9Z,12Z)-octadeca-9,12-dien-1-yl) tetracosa-4,15,18-trien-1-amine (“HGT5001”), having a compound structure of:

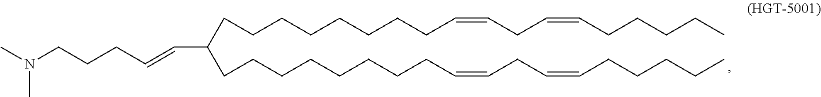 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In some embodiments, the compositions and methods of the present disclosure include the cationic lipid and (15Z,18Z)-N,N-dimethyl-6-((9Z,12Z)-octadeca-9,12-dien-1-yl) tetracosa-5,15,18-trien-1-amine (“HGT5002”), having a compound structure of:

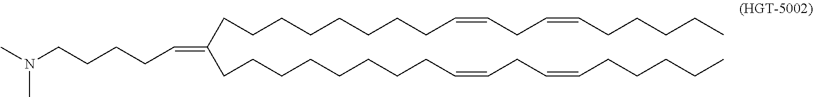 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the disclosure include cationic lipids described as aminoalcohol lipidoids in International Patent Publication WO 2010/053572, which is incorporated herein by reference. In some embodiments, the compositions and methods of the present disclosure include a cationic lipid having a compound structure of:

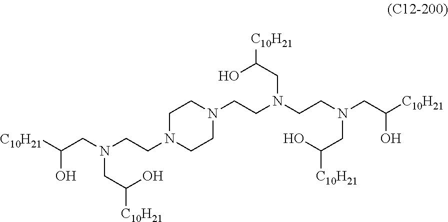

and pharmaceutically acceptable salts thereof.

Other suitable cationic lipids for use in the compositions and methods of the disclosure include the cationic lipids as described in International Patent Publication WO 2016/118725, which is incorporated herein by reference. In some embodiments, the compositions and methods of the present disclosure include a cationic lipid having a compound structure of:

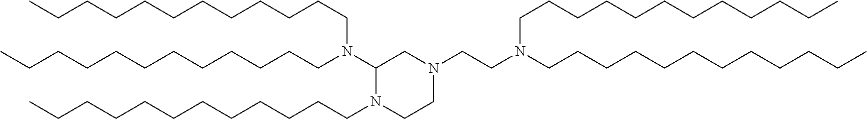 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the disclosure include the cationic lipids as described in International Patent Publication WO 2016/118724, which is incorporated herein by reference. In some embodiments, the compositions and methods of the present disclosure include a cationic lipid having a compound structure of:

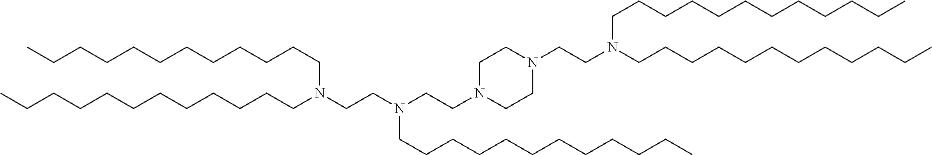 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the disclosure include a cationic lipid having the formula of 14,25-ditridecyl 15,18,21,24-tetraaza-octatriacontane, and pharmaceutically acceptable salts thereof.

Other suitable cationic lipids for use in the compositions and methods of the disclosure include the cationic lipids as described in International Patent Publications WO 2013/063468 and WO 2016/205691, each of which are incorporated herein by reference. In some embodiments, the compositions and methods of the present disclosure include a cationic lipid of the following formula:

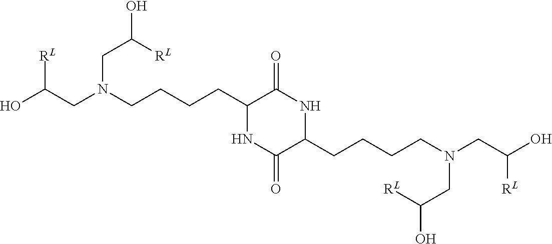

or pharmaceutically acceptable salts thereof, wherein each instance of R^{L} is independently optionally substituted C₆-C₄₀ alkenyl. In some embodiments, the compositions and methods of the present disclosure include a cationic lipid having a compound structure of:

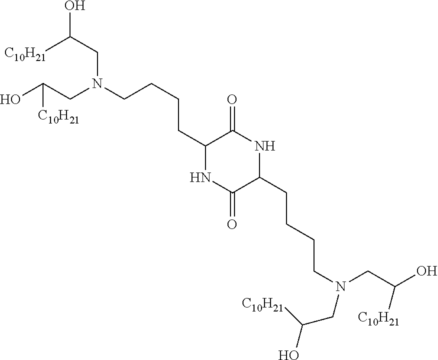

and pharmaceutically acceptable salts thereof. In some embodiments, the compositions and methods of the present disclosure include a cationic lipid having a compound structure of:

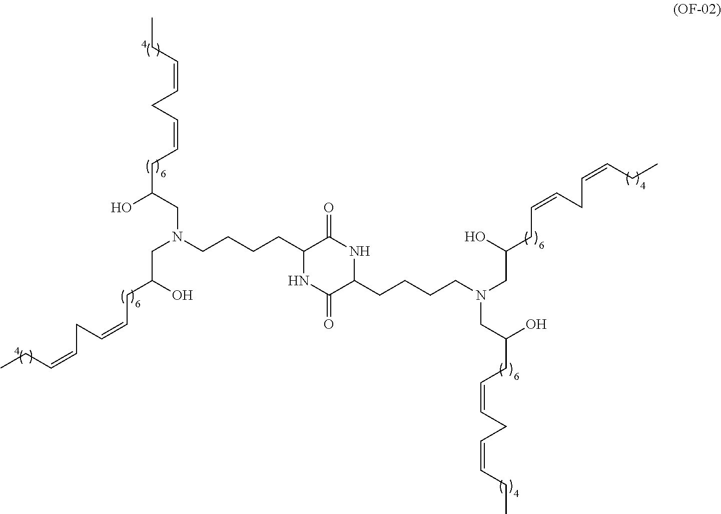

and pharmaceutically acceptable salts thereof. In some embodiments, the compositions and methods of the present disclosure include a cationic lipid having a compound structure of:

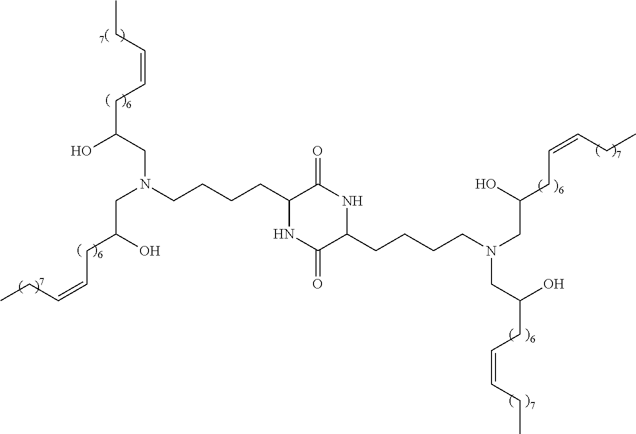

and pharmaceutically acceptable salts thereof. In some embodiments, the compositions and methods of the present disclosure include a cationic lipid having a compound structure of:

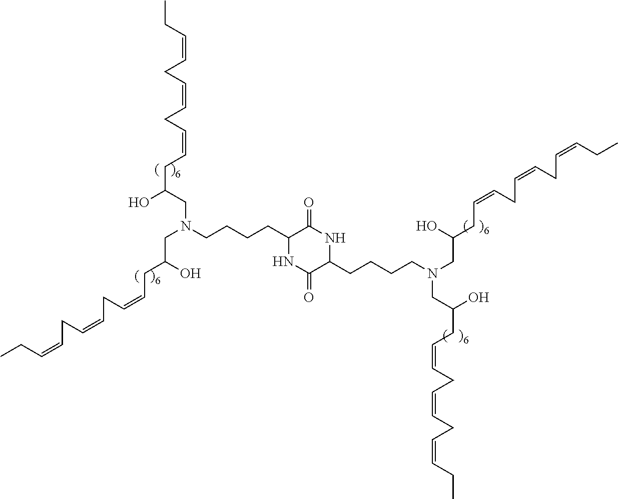

and pharmaceutically acceptable salts thereof.

Other suitable cationic lipids for use in the compositions and methods of the disclosure include the cationic lipids as described in International Patent Publication WO 2015/184256, which is incorporated herein by reference. In some embodiments, the compositions and methods of the present disclosure include a cationic lipid of the following formula:

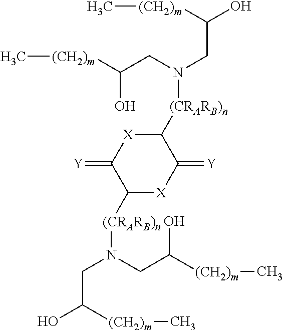

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In some embodiments, the compositions and methods of the present disclosure include a cationic lipid, “Target 23”, having a compound structure of:

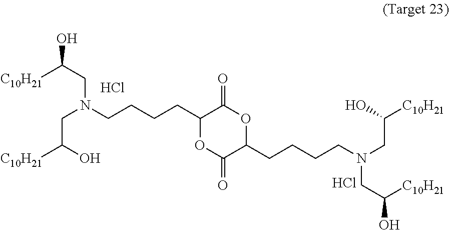

and pharmaceutically acceptable salts thereof.

Other suitable cationic lipids for use in the compositions and methods of the disclosure include the cationic lipids as described in International Patent Publication WO 2016/004202, which is incorporated herein by reference. In some embodiments, the compositions and methods of the present disclosure include a cationic lipid having the compound structure:

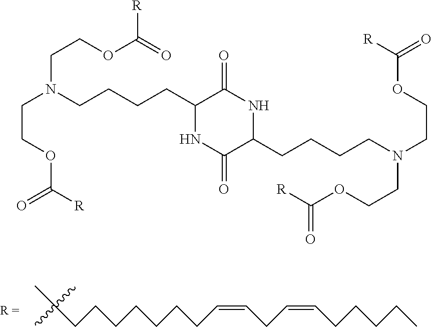

or a pharmaceutically acceptable salt thereof. In some embodiments, the compositions and methods of the present disclosure include a cationic lipid having the compound structure:

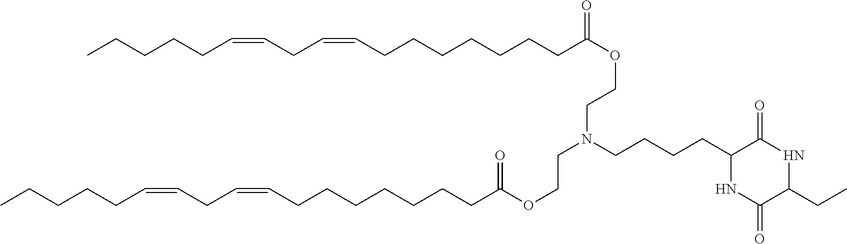 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present disclosure include a cationic lipid having the compound structure:

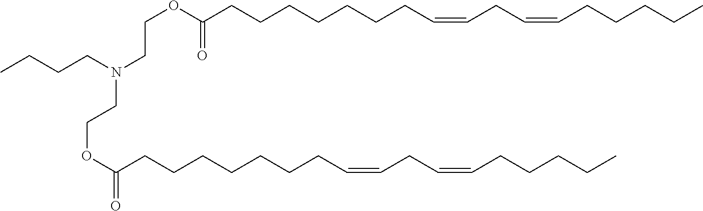 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present disclosure include cationic lipids as described in United States Provisional Patent Application Ser. Number 62/758,179, which is incorporated herein by reference. In some embodiments, the compositions and methods of the present disclosure include a cationic lipid of the following formula:

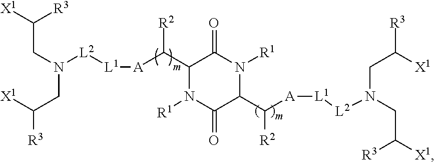

or a pharmaceutically acceptable salt thereof, wherein each R¹ and R² is independently H or C₁-C₆ aliphatic; each m is independently an integer having a value of 1 to 4; each A is independently a covalent bond or arylene; each L₁ is independently an ester, thioester, disulfide, or anhydride group; each L₂ is independently C₂-C₁₀ aliphatic; each X¹ is independently H or OH; and each R³ is independently C₆-C₂₀ aliphatic. In some embodiments, the compositions and methods of the present disclosure include a cationic lipid of the following formula:

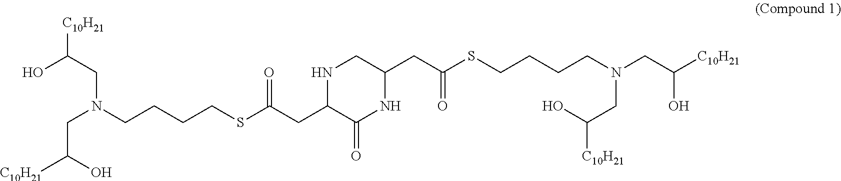

or a pharmaceutically acceptable salt thereof. In some embodiments, the compositions and methods of the present disclosure include a cationic lipid of the following formula:

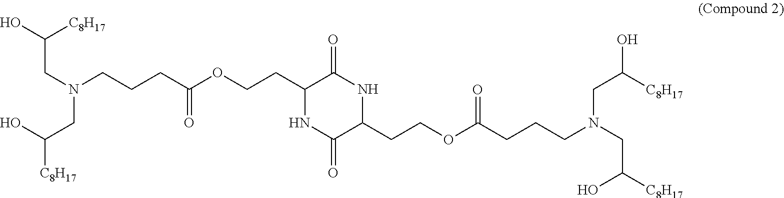

or a pharmaceutically acceptable salt thereof. In some embodiments, the compositions and methods of the present disclosure include a cationic lipid of the following formula:

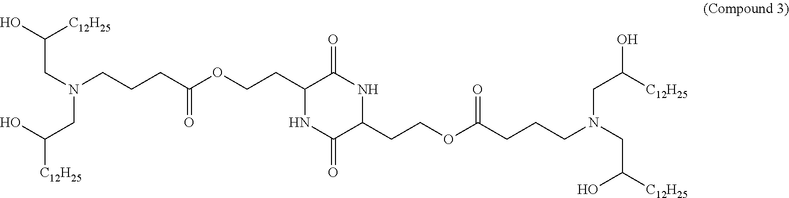

or a pharmaceutically acceptable salt thereof.

Other suitable cationic lipids for use in the compositions and methods of the present disclosure include the cationic lipids as described in J. Mcclellan, M. C. King, Cell 2010, 141, 210-217 and in Whitehead et al., Nature Communications (2014) 5:4277, which is incorporated herein by reference. In some embodiments, the cationic lipids of the compositions and methods of the present disclosure include a cationic lipid having a compound structure of:

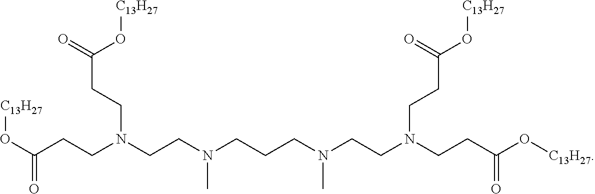

and pharmaceutically acceptable salts thereof.

Other suitable cationic lipids for use in the compositions and methods of the disclosure include the cationic lipids as described in International Patent Publication WO 2015/199952, which is incorporated herein by reference. In some embodiments, the compositions and methods of the present disclosure include a cationic lipid having the compound structure:

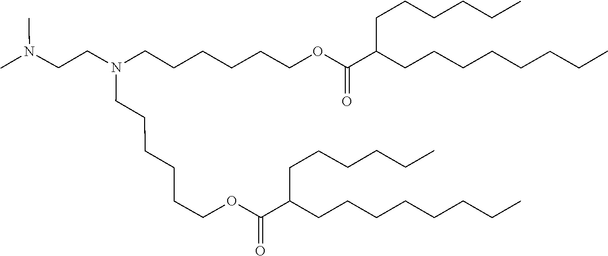 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present disclosure include a cationic lipid having the compound structure:

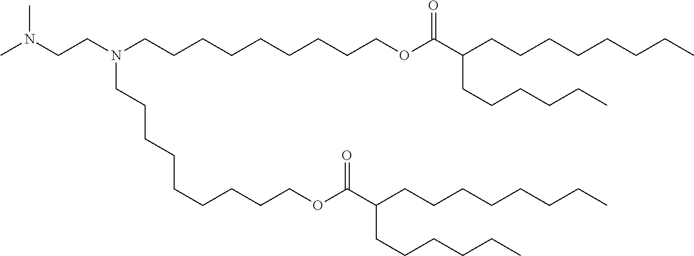 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present disclosure include a cationic lipid having the

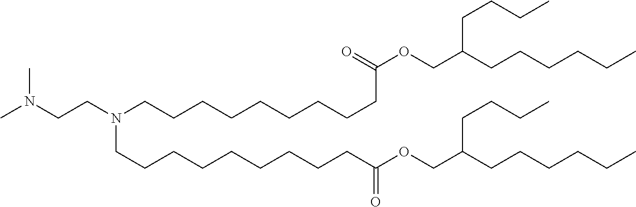 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present disclosure include a cationic lipid having the compound structure:

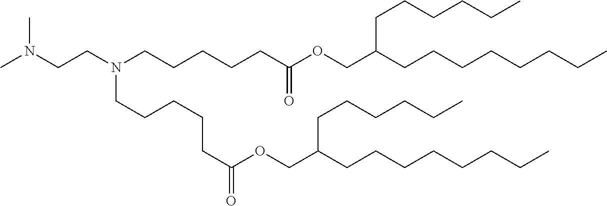 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present disclosure include a cationic lipid having the compound structure:

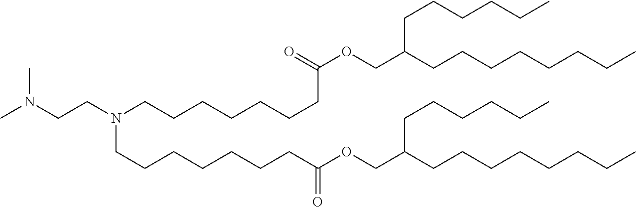 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present disclosure include a cationic lipid having the

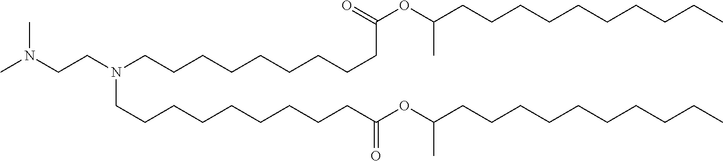 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present disclosure include a cationic lipid having the compound structure:

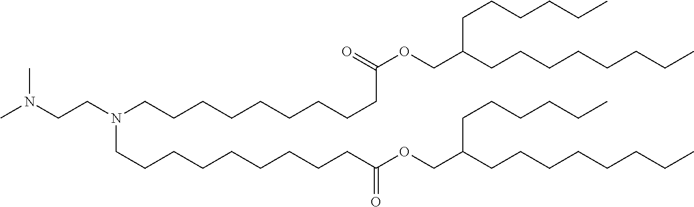 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present disclosure include a cationic lipid having the compound structure:

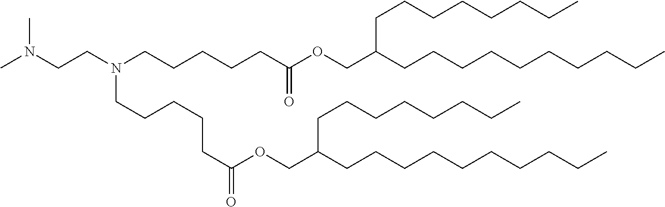 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present disclosure include a cationic lipid having the compound structure:

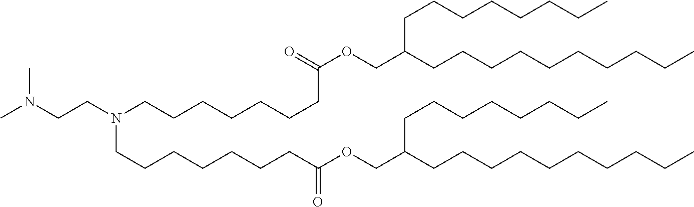 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present disclosure include a cationic lipid having the

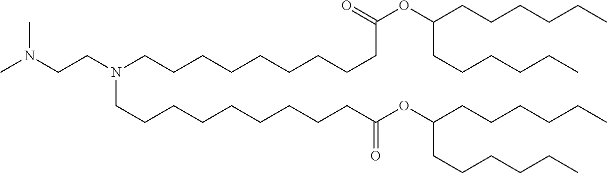 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present disclosure include a cationic lipid having the compound structure:

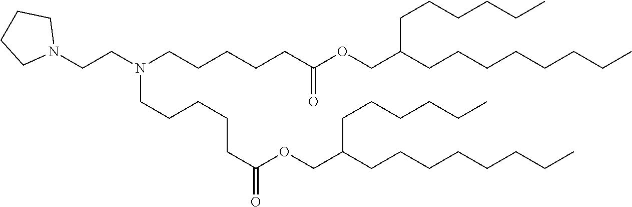 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present disclosure include a cationic lipid having the compound structure:

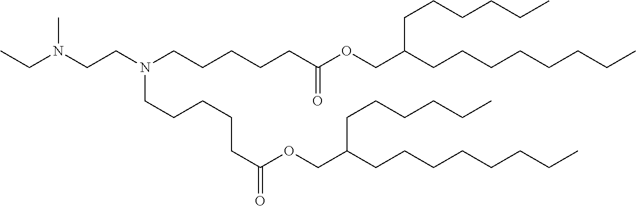 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present disclosure include a cationic lipid having the

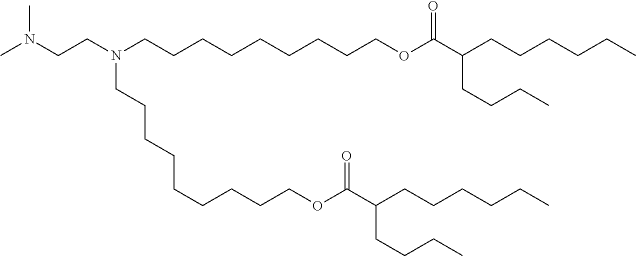 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the disclosure include the cationic lipids as described in International Patent Publication WO 2017/004143, which is incorporated herein by reference. In some embodiments, the compositions and methods of the present disclosure include a cationic lipid having the compound structure:

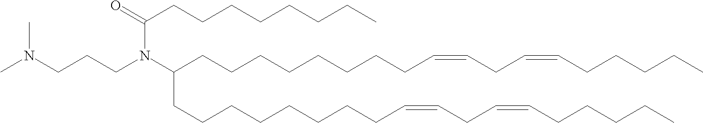 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present disclosure include a cationic lipid having the compound structure:

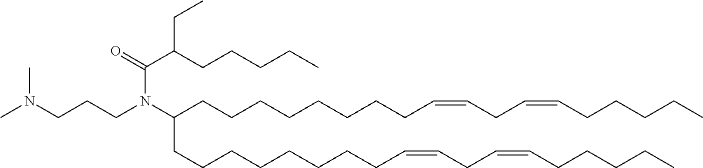 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present disclosure include a cationic lipid having the compound structure:

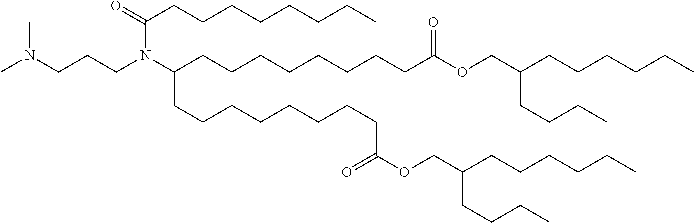 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present disclosure include a cationic lipid having the

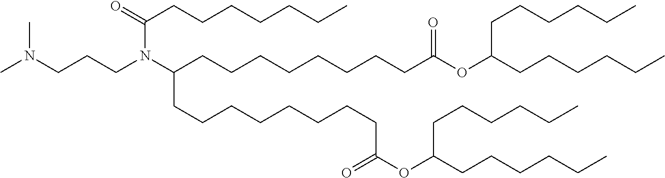 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present disclosure include a cationic lipid having the compound structure:

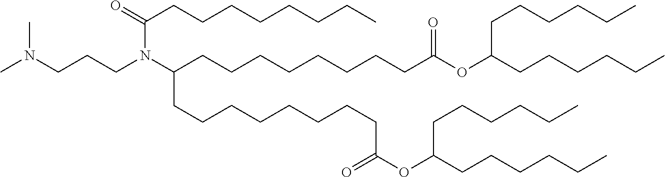 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present disclosure include a cationic lipid having the compound structure:

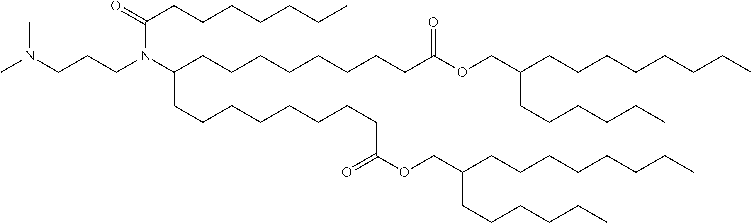 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present disclosure include a cationic lipid having the compound structure:

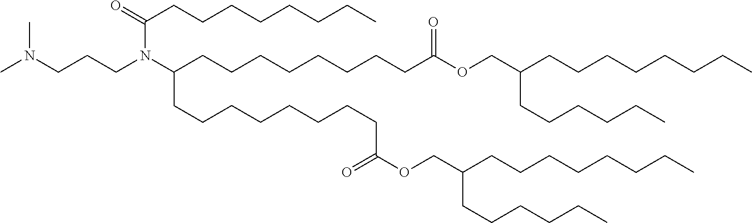 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present disclosure include a cationic lipid having the

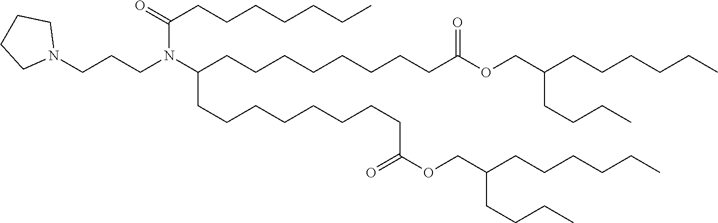 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present disclosure include a cationic lipid having the compound structure:

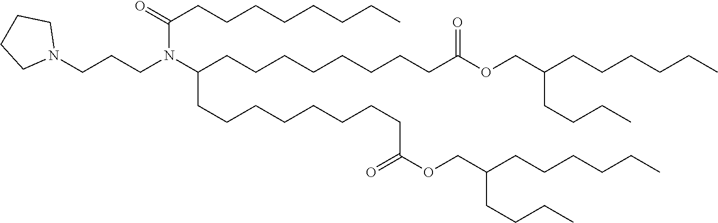 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present disclosure include a cationic lipid having the compound structure:

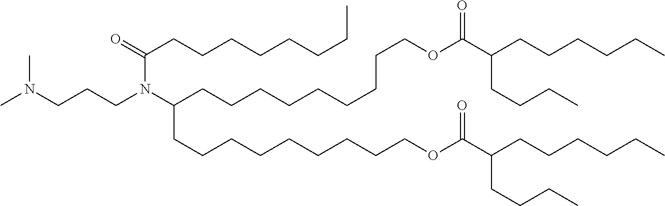 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present disclosure include a cationic lipid having the compound structure:

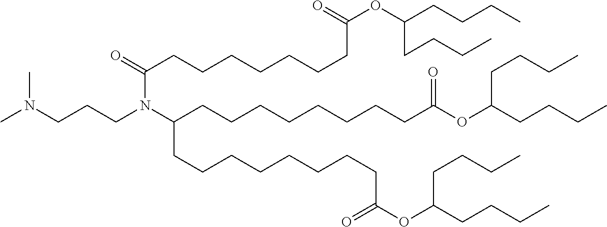 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present disclosure include a cationic lipid having the

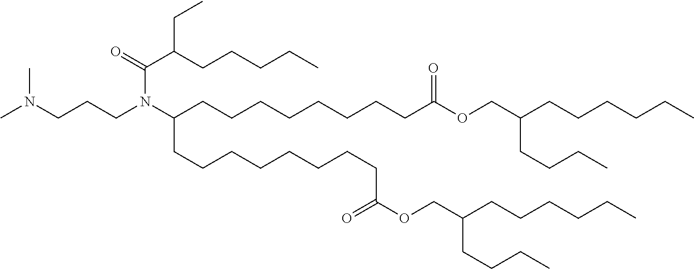 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present disclosure include a cationic lipid having the compound structure:

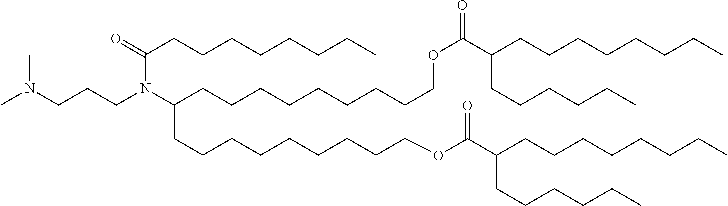 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present disclosure include a cationic lipid having the compound structure:

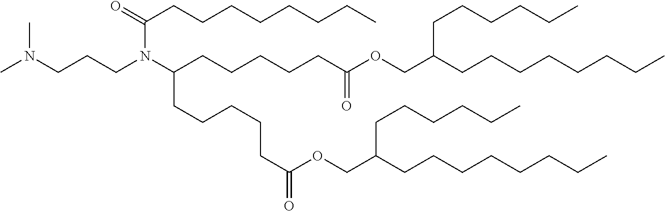 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present disclosure include a cationic lipid having the

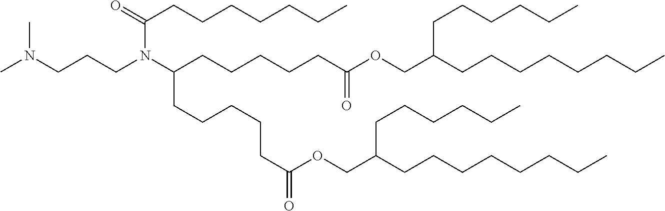 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present disclosure include a cationic lipid having the compound structure:

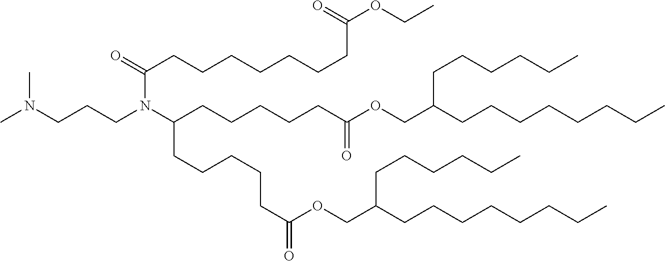 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present disclosure include a cationic lipid having the compound structure:

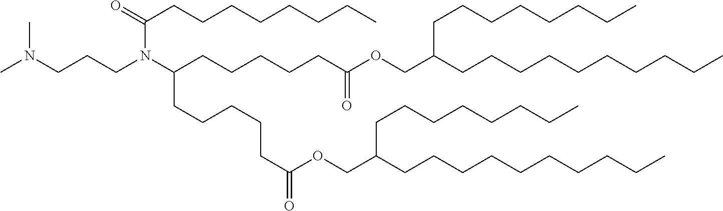 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the disclosure include the cationic lipids as described in International Patent Publication WO 2017/075531, which is incorporated herein by reference. In some embodiments, the compositions and methods of the present disclosure include a cationic lipid of the following formula:

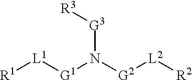

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L′ is —O(C—O)—, —(C═O)O—, —C(═O)—, —O—, —S(O)ₓ, —S—S—, —C(═O)S—, SC(═O)—, —NR^{a}C(═O)—, —C(═O)NR^{a}—,,NR^{a}C(═O)NR^{a}—, —OC(═O)NR^{a}— or—NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the compositions and methods of the disclosure include the cationic lipids as described in International Patent Publication WO 2017/117528, which is incorporated herein by reference.

In some embodiments, the compositions and methods of the present disclosure include a cationic lipid having the compound structure:

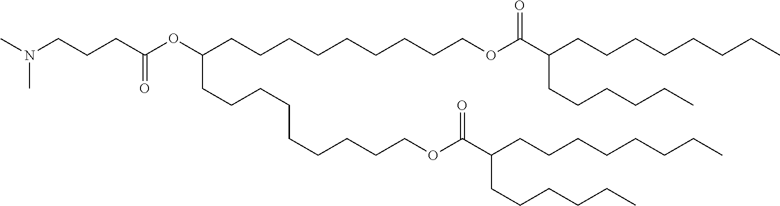 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof.

In some embodiments, the compositions and methods of the present disclosure include a cationic lipid having the compound structure:

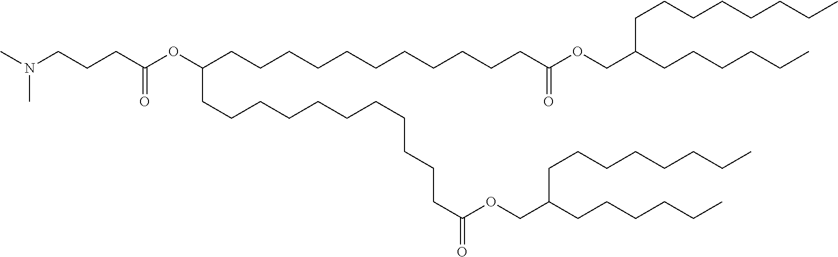 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof.

In some embodiments, the compositions and methods of the present disclosure include a cationic lipid having the compound structure:

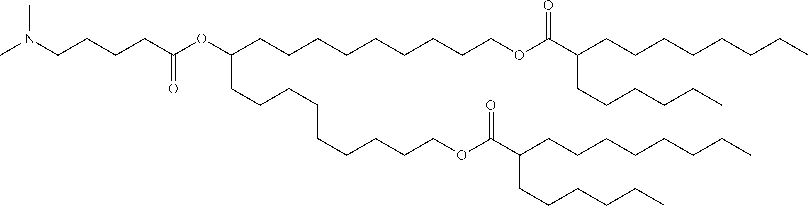 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the disclosure include the cationic lipids as described in International Patent Publication WO 2017/049245, which is incorporated herein by reference.

In some embodiments, the cationic lipids of the compositions and methods of the present disclosure include a compound of one of the following formulas:

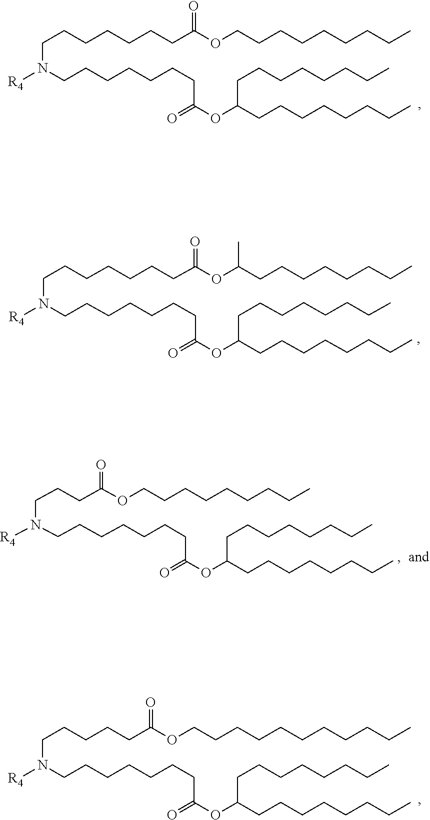

and pharmaceutically acceptable salts thereof.

For any one of these four formulas, R⁴ is independently selected from —(CH₂)ₙQ and —(CH₂)ₙCHQR; Q is selected from the group consisting of—OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, N(H)C(S)N(R)₂, —N(H)C(S)N(H)(R), and a heterocycle; and n is 1, 2, or 3.

In some embodiments, the compositions and methods of the present disclosure include a cationic lipid having a compound structure of:

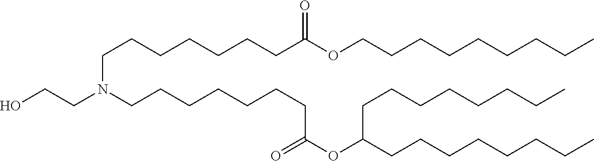 [SMILES: CCCCCCCCCOC(=O)CCCCCCCN(CCO)CCCCCCCC(=O)OC(CCCCCCCC)CCCCCCCC]

and pharmaceutically acceptable salts thereof.

In some embodiments, the compositions and methods of the present disclosure include a cationic lipid having a compound structure of:

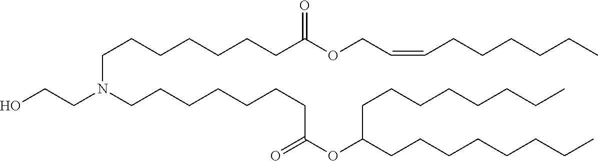 [SMILES: CCCCCC/C=C\COC(=O)CCCCCCCN(CCO)CCCCCCCC(=O)OC(CCCCCCCC)CCCCCCCC]

and pharmaceutically acceptable salts thereof.

In some embodiments, the compositions and methods of the present disclosure include a cationic lipid having a compound structure of:

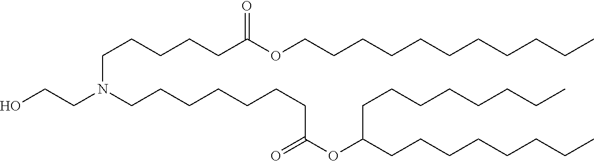 [SMILES: CCCCCCCCCCCOC(=O)CCCCCN(CCO)CCCCCCCC(=O)OC(CCCCCCCC)CCCCCCCC]

and pharmaceutically acceptable salts thereof.

In certain some embodiments, the compositions and methods of the present disclosure include a cationic lipid having a compound structure of:

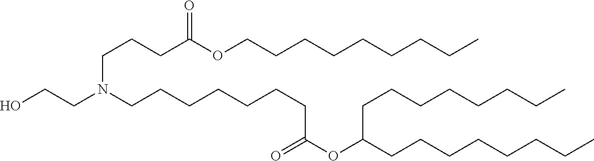 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the disclosure include the cationic lipids as described in International Patent Publication WO 2017/173054 and WO 2015/095340, each of which is incorporated herein by reference.

In certain some embodiments, the compositions and methods of the present disclosure include a cationic lipid having a compound structure of:

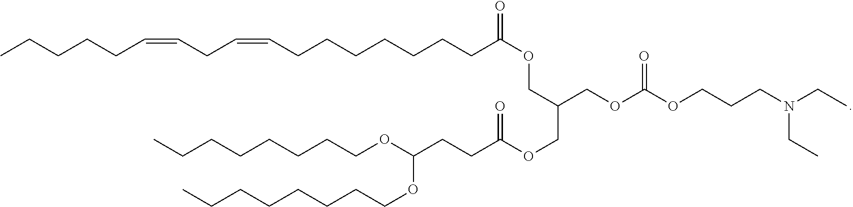 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof.

In some embodiments, the compositions and methods of the present disclosure include a cationic lipid having a compound structure of:

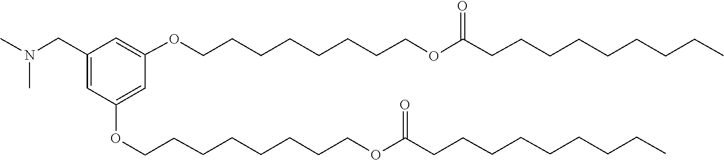 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof.

In some embodiments, the compositions and methods of the present disclosure include a cationic lipid having a compound structure of:

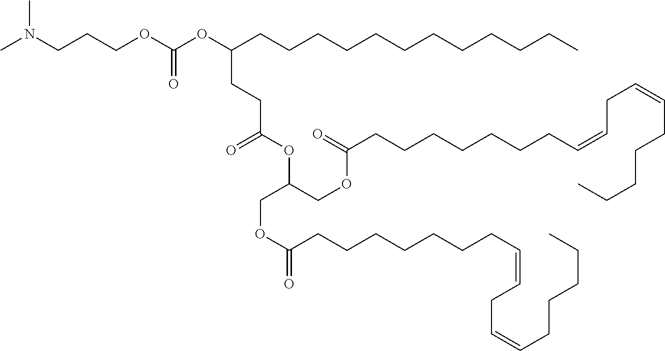 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof.

In some embodiments, the compositions and methods of the present disclosure include a cationic lipid having a compound structure of:

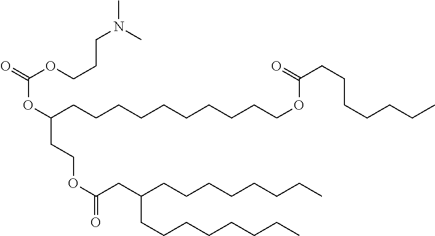 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present disclosure include cleavable cationic lipids as described in International Patent Publication WO 2012/170889, which is incorporated herein by reference. In some embodiments, the compositions and methods of the present disclosure include a cationic lipid of the following formula:

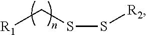

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

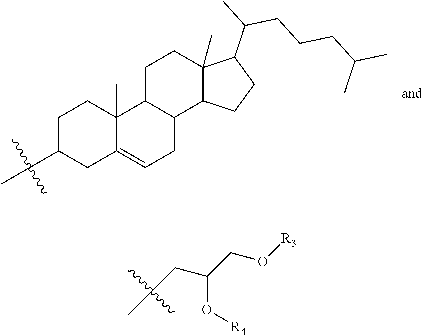

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more).

In some embodiments, the compositions and methods of the present disclosure include a cationic lipid, “HGT4001”, having a compound structure of:

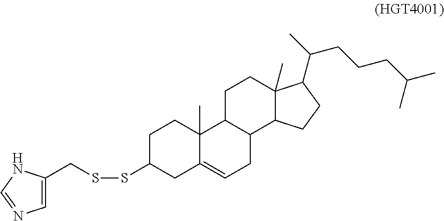 [SMILES: CC(C)CCCC(C)C1CCC2C3CC=C4CC(SSCc5cnc[nH]5)CCC4(C)C3CCC12C]

and pharmaceutically acceptable salts thereof.

In some embodiments, the compositions and methods of the present disclosure include a cationic lipid, “HGT4002”, having a compound structure of:

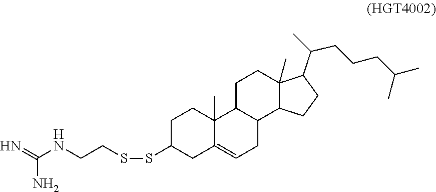 [SMILES: CC(C)CCCC(C)C1CCC2C3CC=C4CC(SSCCNC(=N)N)CCC4(C)C3CCC12C]

and pharmaceutically acceptable salts thereof.

In some embodiments, the compositions and methods of the present disclosure include a cationic lipid, “HGT4003”, having a compound structure of:

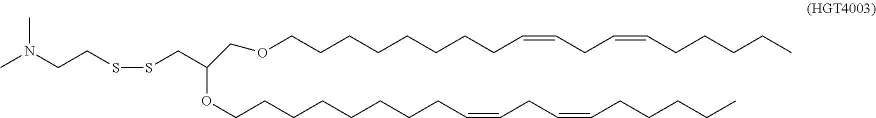 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof.

In some embodiments, the compositions and methods of the present disclosure include a cationic lipid, “HGT4004”, having a compound structure of:

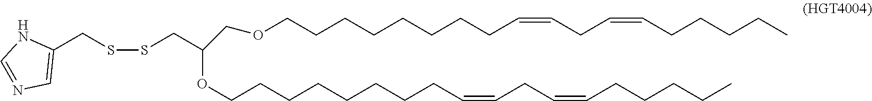 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof.

In some embodiments, the compositions and methods of the present disclosure include a cationic lipid “HGT4005”, having a compound structure of:

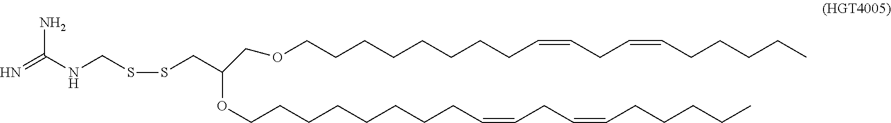 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present disclosure include HEPES-based disulfide cationic lipids with a piperazine core as described in International Patent Publication WO 2022/221688, which is incorporated herein by reference. In some embodiments, the compositions and methods of the present disclosure include a cationic lipid of the following formula:

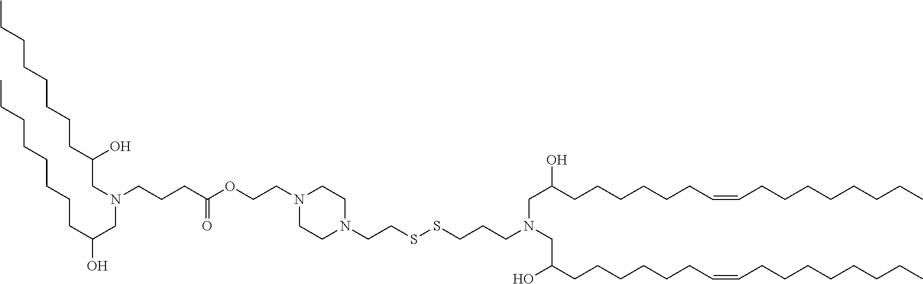 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

(GL-HEPES-E3-E10-DS-3-E18-1:(2-(4-(2-((3-(Bis((Z)-2-hydroxyoctadec-9-en-1-yl)amino) propyl)disulfaneyl)ethyl)piperazin-1-yl)ethyl 4-(bis(2-hydroxydecyl)amino)butanoate)) and pharmaceutically acceptable salts thereof. In some embodiments, the compositions and methods of the present disclosure include a cationic lipid of the following formula:

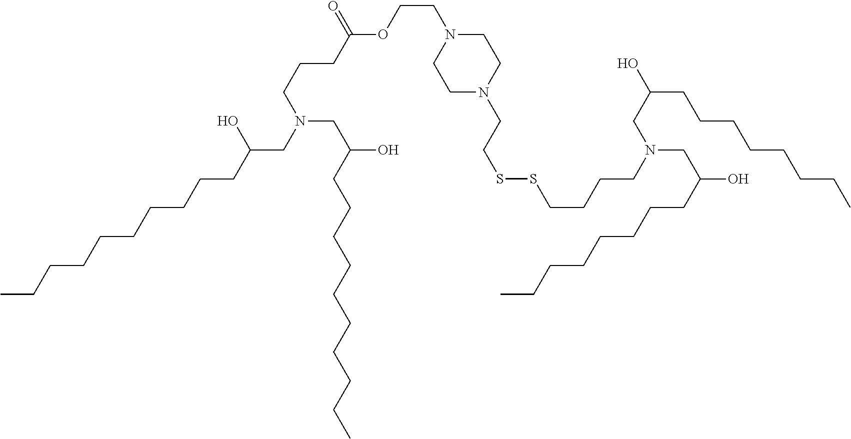 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

(GL-HEPES-E3-E12-DS-4-E10: (2-(4-(2-((3-(bis(2-hydroxydecyl)amino)butyl)disulfaneyl)ethyl)piperazin-1-yl)ethyl 4-(bis(2-hydroxydodecyl)amino)butanoate)) and pharmaceutically acceptable salts thereof. In some embodiments, the compositions and methods of the present disclosure include a cationic lipid of the following formula:

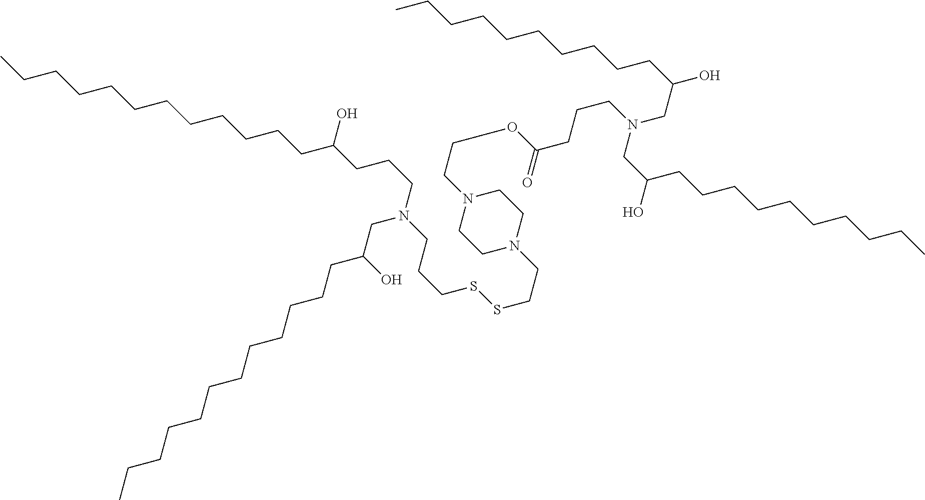 [SMILES: CCCCCCCCCCCCC(O)CCCN(CCCSSCCN1CCN(CCOC(=O)CCCN(CC(O)CCCCCCCCCC)CC(O)CCCCCCCCCC)CC1)CC(O)CCCCCCCCCCCC]

(GL-HEPES-E3-E12-DS-3-E14: (2-(4-(2-((3-(Bis(2-hydroxytetradecyl)amino) propyl) disulfaneyl)ethyl)piperazin-1-yl)ethyl 4-(bis(2-hydroxydodecyl)amino)butanoate)) and pharmaceutically acceptable salts thereof.

Other suitable cationic lipids for use in the compositions and methods of the present disclosure include cationic lipids as described in Dong et al., PNAS, 2014, 111 (11): 3955-3960 and U.S. Pat. No. 9,512,073, which are incorporated herein by reference. In some embodiments, the compositions and methods of the present disclosure include a cationic lipid of the following formula:

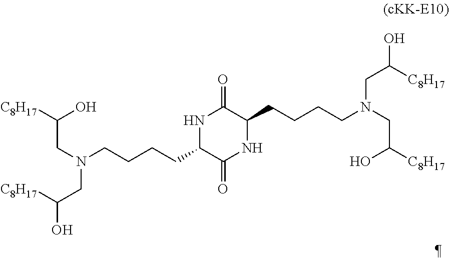

and pharmaceutically acceptable salts thereof. In some embodiments, the compositions and methods of the present disclosure include a cationic lipid of the following formula:

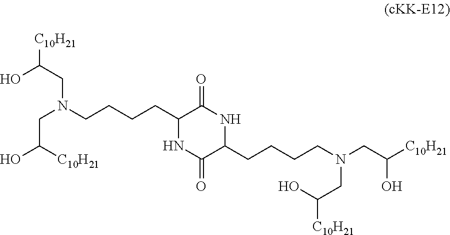

and pharmaceutically acceptable salts thereof.

Other suitable cationic lipids for use in the compositions and methods of the present disclosure include cleavable cationic lipids as described in U.S. Provisional Application No. 62/672,194, filed May 16, 2018, and incorporated herein by reference. In some embodiments, the compositions and methods of the present disclosure include a cationic lipid that is any of general formulas or any of structures (1a)-(21a) and (1b)-(21b) and (22)-(237) described in U.S. Provisional Application No. 62/672,194.

In some embodiments, the compositions and methods of the present disclosure include a cationic lipid that has a structure according to Formula (I′),

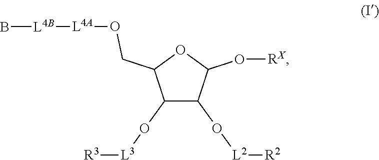

wherein:
R^{X} is independently —H, —L¹-R¹, or −L⁵A-L⁵B—B′;
each of L¹, L², and L³ is independently a covalent bond, —C(O)—, —C(O)O—, —C(O)S—, or —C(O)NR^{L}-;
each L⁴A and L⁵A is independently —C(O)—, —C(O)O—, or —C(O)NR^{L}-;
each L⁴B and L⁵B is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl;
and
each R^{L} is independently hydrogen, C₁-C₂₀ alkyl, C₂-C₂₀ alkenyl, or C₂-C₂₀ alkynyl.

In some embodiments, the compositions and methods of the present disclosure include a cationic lipid that is Compound (139) of 62/672,194, having a compound structure of:

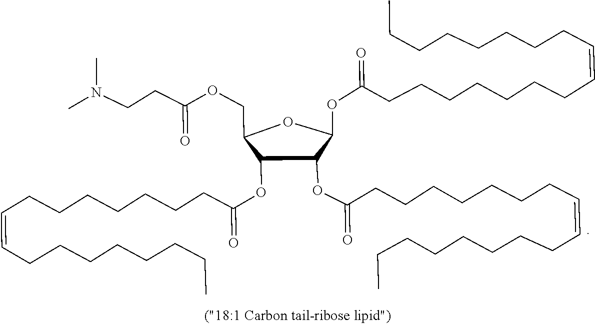

In some embodiments, the compositions and methods of the present disclosure include the cationic lipid, N-[1-(2,3-dioleyloxy) propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present disclosure include, for example, 5-carboxyspermylglycinedioctadecylamide (“DOGS”); 2,3-dioleyloxy-N-[2 (spermine-carboxamido)ethyl]-N,N-dimethyl-1-propanaminium (“DOSPA”) (Behr et al. Proc. Nat.′1 Acad. Sci. 86, 6982 (1989), U.S. Pat. Nos. 5,171,678; and 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present disclosure also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis -9,12-octadecadienoxy) propane (“CLinDMA”); 2-[5′-(cholest-5-en-3-beta-oxy)-3′-oxapentoxy)-3-dimethy 1-1-(cis,cis -9′, 1-2′-octadecadienoxy) propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z,12Z)-octadeca-9,12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z,12Z)-octadeca-9, 12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z,12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,1 2-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28:172-176 (2010)). (Heyes, J., et al., J Controlled Release 107:276-287 (2005); Morrissey, D V., et al., Nat. Biotechnol. 23 (8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present disclosure include 2,2-Dilinoleyl-4-dimethylaminoethyl-[1,3]-dioxolane (“XTC”); (3aR,5s,6aS)—N,N-dimethyl-2,2-di((9Z,12Z)-octadeca-9,12-dienyl)tetrahydro-3aH-cyclopenta[d][1,3]dioxol-5-amine (“ALNY-100”) and/or 4,7,13-tris(3-oxo-3-(undecylamino) propyl)-N1,N16-diundecyl-4,7,10,13-tetraazahexadecane-1,16-diamide (“NC98-5”).

In some embodiments, the compositions and methods of the present disclosure include the cationic lipid known as ALC-0315 ([(4-hydroxybutyl) azanediyl]di(hexane-6,1-diyl)bis(2-hexyldecanoate)), which is a synthetic lipid having the following chemical structure:

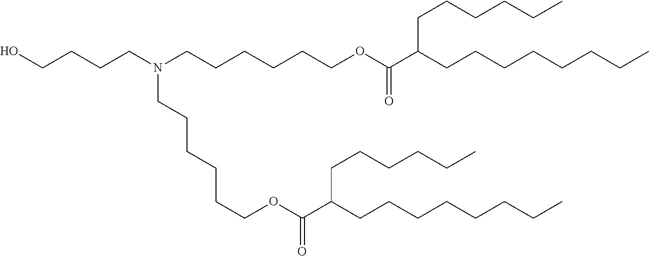 [SMILES: CCCCCCCCC(CCCCCC)C(=O)OCCCCCCN(CCCCO)CCCCCCOC(=O)C(CCCCCC)CCCCCCCC]

and pharmaceutically acceptable salts thereof.

In some embodiments, the compositions of the present disclosure include one or more cationic lipids that constitute at least about 5%, 10%, 20%, 30%, 35%, 40%, 45%, 50%, 55%, 60%, 65%, or 70%, including any values and subranges therebetween, measured by weight, of the total lipid content in the composition, e.g., a lipid nanoparticle. In some embodiments, the compositions of the present disclosure include one or more cationic lipids that constitute at least about 5%, 10%, 20%, 30%, 35%, 40%, 45%, 50%, 55%, 60%, 65%, or 70%, including any values and subranges therebetween, measured as a mol %, of the total lipid content in the composition, e.g., a lipid nanoparticle. In some embodiments, the compositions of the present disclosure include one or more cationic lipids that constitute about 30-70% (e.g., about 30-65%, about 30-60%, about 30-55%, about 30-50%, about 30-45%, about 30-40%, about 35-50%, about 35-45%, or about 35-40%, including any values and subranges therebetween), measured by weight, of the total lipid content in the composition, e.g., a lipid nanoparticle. In some embodiments, the compositions of the present disclosure include one or more cationic lipids that constitute about 30-70% (e.g., about 30-65%, about 30-60%, about 30-55%, about 30-50%, about 30-45%, about 30-40%, about 35-50%, about 35-45%, or about 35-40%, including any values and subranges therebetween), measured as mol %, of the total lipid content in the composition, e.g., a lipid nanoparticle.

### Non-Cationic/Helper Lipids

In some embodiments, provided liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, a non-cationic lipid is DPPC. In some embodiments, a non-cationic lipid is DOPE. In some embodiments, a non-cationic lipid is DEPE.

In some embodiments, such non-cationic lipids may be used alone, but are preferably used in combination with other lipids, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

### Cholesterol-Based Lipids

In some embodiments, provided liposomes comprise one or more cholesterol-based lipids. For example, suitable cholesterol-based lipids include cholesterol and, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, the cholesterol-based lipid may comprise a molar ratio of about 2% to about 30%, or about 5% to about 20% of the total lipid present in a liposome. In some embodiments, the percentage of cholesterol-based lipid in the lipid nanoparticle may be greater than 5%, greater than 10%, greater than 20%, greater than 30%, or greater than 40%.

### PEG-Modified Lipids

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C₈ PEG-2000 ceramide) is also contemplated by the present disclosure, either alone or preferably in combination with other lipid formulations together which comprise the transfer vehicle (e.g., a lipid nanoparticle). Contemplated PEG-modified lipids include, but are not limited to, a polyethylene glycol chain of up to S kDa in length covalently attached to a lipid with alkyl chain(s) of C₆-C₂₀ length. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids of the present disclosure may comprise a molar ratio from about 0% to about 20%, about 0.5% to about 20%, about 1% to about 15%, about 4% to about 10%, or about 2% of the total lipid present in the liposomal transfer vehicle. In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio.

### Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

Exemplary combinations of cationic lipids, non-cationic lipids, cholesterol-based lipids, and PEG-modified lipids are described in the Examples section. For example, a suitable lipid solution may contain CKK-E10, DOPE, cholesterol, and DMG-PEG2K; CKK-E12, DOPE, cholesterol, and DMG-PEG2K; C12-200, DOPE, cholesterol, and DMG-PEG2K; HGT5000, DOPE, cholesterol, and DMG-PEG2K; HGT5001, DOPE, cholesterol, and DMG-PEG2K; OF-02, DOPE, cholesterol, and DMG-PEG2K; GL-HEPES-E3-E12-DS-4-E10, DOPE, cholesterol, and DMG-PEG2K; CKK-E10, DPPC, cholesterol, and DMG-PEG2K; CKK-E12, DPPC, cholesterol, and DMG-PEG2K; C 12-200, DPPC, cholesterol, and DMG-PEG2K; HGT5000, DPPC, cholesterol, and DMG-PEG2K; HGT5001, DPPC, cholesterol, and DMG-PEG2K; OF-02, DPPC, cholesterol, and DMG-PEG2K; or GL-HEPES-E3-E12-DS-4-E10, cholesterol, and DMG-PEG2K. The selection of cationic lipids, non-cationic lipids and/or PEG-modified lipids which comprise the lipid mixture as well as the relative molar ratio of such lipids to each other, is based upon the characteristics of the selected lipid(s) and the nature of the and the characteristics of the mRNA to be encapsulated. Additional considerations include, for example, the saturation of the alkyl chain, as well as the size, charge, pH, pKa, fusogenicity and toxicity of the selected lipid(s). Thus, the molar ratios may be adjusted accordingly.

In some embodiments, the LNPs are manufactured with a particular molar ratio of cationic lipids to non-cationic lipids to cholesterol-based lipids to PEG-modified lipids. In some embodiments, lipid nanoparticle comprises a molar ratio of cationic lipids to non-cationic lipids to cholesterol-based lipids to PEG-modified lipids of 60:25:10:5. In some embodiments, lipid nanoparticle comprises a molar ratio of cationic lipids to non-cationic lipids to cholesterol-based lipids to PEG-modified lipids of 40:25:30:5.

### Therapeutic Uses

In some embodiments, a dry powder formulation (e.g., a reconstitutable dry powder formulation) or a reconstituted dry powder formulation described herein comprises any full-length mRNA. In some embodiments, a formulation described herein comprises mRNA encoding for a drug or a peptide or protein therapeutic suitable for the present disclosure. In some embodiments, a formulation described herein comprises a full-length mRNA that encodes for any peptide or polypeptide suitable for the present disclosure. In some embodiments, a formulation is a dry powder formulation (e.g., a reconstitutable dry powder formulation). In some embodiments, a formulation is a reconstituted formulation (e.g., reconstituted from a reconstitutable dry powder formulation as described herein). In some embodiments, a formulation described herein comprises other nucleic acids for in vivo administration, as discussed herein.

Exemplary, non-limiting mRNAs suitable for use in dry powder and reconstituted LNP formulations are described herein.

Therapeutic Proteins Encoded by the mRNA:

### Exemplary Naturally Occurring and Engineered Proteins and Peptides:

In some embodiments, a formulation described herein comprises a full-length mRNA that encodes for any antibody suitable for the present disclosure. In some embodiments, a formulation described herein comprises a full-length mRNA that encodes for any therapeutic protein suitable for the present disclosure. In some embodiments, a formulation described herein comprises a full-length mRNA that encodes for any naturally occurring peptide suitable for the present disclosure. In some embodiments, a formulation described herein comprises a full-length mRNA that encodes for any modified or non-naturally occurring peptide suitable for the present disclosure. In some embodiments, a formulation described herein comprises a full-length mRNA that encodes for any peptide drug suitable for the present disclosure.

In some embodiments, an mRNA encodes one or more naturally occurring peptides. In some embodiments, an mRNA encodes one or more modified or non-natural peptides.

In some embodiments, the peptide drug include, glucose-dependent insulinotropic polypeptide. A further example of the peptide drug is elamipretide. Further examples of the peptide drug are cyclotides (which are peptides characterized by their head-to-tail cyclized peptide backbone and the interlocking arrangement of their disulfide bonds), including, e.g., a cyclotide having at least two disulfide bonds (and preferably a cyclotide having three disulfide bonds).

In some embodiments, the peptide drug is selected from GLP-1, amylin, an amylin analog, pramlintide, a somatostatin analog (e.g., octreotide, lanreotide, or pasireotide), goserelin (e.g., goserelin acetate), buserelin, peptide YY (PYY), a PYY analog, glatiramer (e.g., glatiramer acetate), leuprolide (e.g., leuprolide acetate), desmopressin (e.g., desmopressin acetate, particularly desmopressin monoacetate trihydrate), teicoplanin, telavancin, bleomycin, ramoplanin, decaplanin, bortezomib, cosyntropin, sermorelin, luteinizing-hormone-releasing hormone (LHRH), calcitonin (e.g., calcitonin-salmon), pentagastrin, neseritide, enfuvirtide, eptifibatide, cyclosporine, glucagon, viomycin, thyrotropin-releasing hormone (TRH), leucine-enkephalin, methionine-enkephalin, substance P, a parathyroid hormone (PTH) fragment (e.g., teriparatide (PTH (1-34)), PTH (1-31), or PTH (2-34)), carfilzomib, icatibant, cilengitide, a prostaglandin F2a receptor modulator (e.g., PDC31), and pharmaceutically acceptable salts thereof. It is particularly preferred that the peptide drug is selected from semaglutide, liraglutide, teriparatide (PTH (1-34)), octreotide, leuprolide, and pharmaceutically acceptable salts thereof.

### Exemplary Proteins for Tissue Specific Delivery

In some embodiments, a formulation described herein comprises a full-length mRNA that encodes for any peptide or polypeptide for use in the delivery to or treatment of the lung of a subject or a lung cell suitable for the present disclosure.

In some embodiments, a formulation described herein comprises a full-length mRNA that encodes for any peptide or polypeptide for use in the delivery to or treatment of the liver of a subject or a liver cell suitable for the present disclosure.

In some embodiments, a formulation described herein comprises a full-length mRNA that encodes for a protein associated with a urea cycle disorder suitable for the present disclosure.

In some embodiments, a formulation described herein comprises a full-length mRNA that encodes for a protein associated with a lysosomal storage disorder suitable for the present disclosure.

In some embodiments, a formulation described herein comprises a full-length mRNA that encodes for a protein associated with a glycogen storage disorder suitable for the present disclosure.

In some embodiments, a formulation described herein comprises a full-length mRNA that encodes for a protein associated with amino acid metabolism suitable for the present disclosure.

In some embodiments, a formulation described herein comprises a full-length mRNA that encodes for a protein associated with a lipid metabolism or fibrotic disorder suitable for the present disclosure.

In some embodiments, a formulation described herein comprises a full-length mRNA that encodes for a protein associated with methylmalonic acidemia suitable for the present disclosure.

In some embodiments, a formulation described herein comprises a full-length mRNA that encodes for a peptide or polypeptide for use in the delivery to or treatment of the cardio-vasculature of a subject or a cardiovascular cell suitable for the present disclosure.

In some embodiments, a formulation described herein comprises a full-length mRNA that encodes for any peptide or polypeptide for use in the delivery to or treatment of the muscle of a subject or a muscle cell suitable for the present disclosure.

In some embodiments, a formulation described herein comprises a full-length mRNA that encodes for a peptide or polypeptide for use in the delivery to or treatment of the nervous system of a subject or a nervous system cell suitable for the present disclosure.

In some embodiments, a formulation described herein comprises a full-length mRNA that encodes for any peptide or polypeptide for use in the delivery to or treatment of the eye of a subject or a eye cell suitable for the present disclosure.

Exemplary embodiments are described herein.

a. Liver

In some embodiments, the present disclosure provides a formulation having full-length mRNA for delivery to the liver. In some embodiments, the dry powder formulation is reconstituted.

In some embodiments, the present disclosure provides a formulation having full-length mRNA for delivery to liver or a formulation having full-length mRNA for treatment of liver-associated condition. In some embodiments the present disclosure provides a formulation having full-length mRNA that encodes for ATP7B protein, also known as Wilson disease protein. In some embodiments, the present disclosure provides a formulation having full-length mRNA that encodes for porphobilinogen deaminase enzyme. In some embodiments the present disclosure provides a formulation having full-length mRNA that encodes for one or clotting enzymes, such as Factor VIII, Factor IX, Factor VII, and Factor X. In some embodiments, the present disclosure provides a formulation having full-length mRNA that encodes for human hemochromatosis (HFE) protein.

b. Lung

In some embodiments, a formulation described herein comprises a full-length mRNA that encodes for a peptide or polypeptide for use in the delivery of or treatment into the lung of a subject or a cell of a subject suitable for the present disclosure. Additional embodiments are described herein.

In some embodiments, the present disclosure provides a formulation comprising full-length mRNA that encodes a peptide or polypeptide for use in the delivery to or treatment of the lung of a subject or a lung cell.

In some embodiments, the present disclosure provides a formulation having full-length mRNA that encodes for cystic fibrosis transmembrane conductance regulator (CFTR) protein. In some embodiments, the present disclosure provides a formulation having full-length mRNA that encodes for ATP-binding cassette sub-family A member 3 protein. In some embodiments, the present disclosure provides a formulation having full-length mRNA that encodes for dynein axonemal intermediate chain 1 protein. In some embodiments, the present disclosure provides a formulation having full-length mRNA that encodes for dynein axonemal heavy chain 5 (DNAH5) protein. In some embodiments, the present disclosure provides a formulation having full-length mRNA that encodes for alpha-1-antitrypsin protein. In some embodiments, the present disclosure provides a formulation having full-length mRNA that encodes for forkhead box P3 (FOXP3) protein. In some embodiments, the present disclosure provides a formulation having full-length mRNA that encodes one or more surfactant protein, e.g., one or more of surfactant A protein, surfactant B protein, surfactant C protein, and surfactant D protein. In some embodiments, dry powder formulations comprising trehalose are used for lung delivery. In some embodiments, the formulations comprising a sugar or sugar alcohol excipients (for example, trehalose) having a particle size less than 5 microns are suited to lung delivery.

### C. Heart

In some embodiments, the present disclosure provides a formulation having full-length mRNA that encodes a peptide or polypeptide for use in the delivery to or treatment of the cardiovasculature of a subject or a cardiovascular cell. In some embodiments, the present disclosure provides a formulation having full-length mRNA that encodes for vascular endothelial growth factor A protein. In some embodiments, the present disclosure provides a formulation having full-length mRNA that encodes for relaxin protein. In some embodiments, the present disclosure provides a formulation having full-length mRNA that encodes for bone morphogenetic protein-9 protein. In some embodiments, the present disclosure provides a formulation having full-length mRNA that encodes for bone morphogenetic protein-2 receptor protein.

d. Muscle

In some embodiments, the present disclosure provides a formulation having full-length mRNA that encodes a peptide or polypeptide for use in the delivery to or treatment of the muscle of a subject or a muscle cell. In some embodiments, the present disclosure provides a formulation having full-length mRNA that encodes for dystrophin protein. In some embodiments, the present disclosure provides a formulation having full-length mRNA that encodes for frataxin protein. In some embodiments, the present disclosure provides a formulation having full-length mRNA that encodes a peptide or polypeptide for use in the delivery to or treatment of the cardiac muscle of a subject or a cardiac muscle cell. In some embodiments, the present disclosure provides a formulation having full-length mRNA that encodes for a protein that modulates one or both of a potassium channel and a sodium channel in muscle tissue or in a muscle cell. In some embodiments, the present disclosure provides a formulation having full-length mRNA that encodes for a protein that modulates a Kv7.1 channel in muscle tissue or in a muscle cell. In some embodiments, the present disclosure provides a formulation having full-length mRNA that encodes for a protein that modulates a Nav1.5 channel in muscle tissue or in a muscle cell.

e. Nerve Cells

In some embodiments, the present disclosure provides a formulation having full-length mRNA that encodes a peptide or polypeptide for use in the delivery to or treatment of the nervous system of a subject or a nervous system cell. For example, in some embodiments, the present disclosure provides a formulation having full-length mRNA that encodes for survival motor neuron 1 protein. For example, in some embodiments, the present disclosure provides a formulation having full-length mRNA that encodes for survival motor neuron 2 protein. In some embodiments, the present disclosure provides a formulation having full-length mRNA that encodes for frataxin protein. In some embodiments, the present disclosure provides a formulation having full-length mRNA that encodes for ATP binding cassette subfamily D member 1 (ABCD1) protein. In some embodiments, the present disclosure provides a formulation having full-length mRNA that encodes for CLN3 protein.

f. Bone Marrow

In some embodiments, the present disclosure provides a formulation having full-length mRNA that encodes a peptide or polypeptide for use in the delivery to or treatment of the blood or bone marrow of a subject or a blood or bone marrow cell. In some embodiments, the present disclosure provides a formulation having full-length mRNA that encodes for beta globin protein. In some embodiments, the present disclosure provides a formulation having full-length mRNA that encodes for Bruton's tyrosine kinase protein. In some embodiments, the present disclosure provides a formulation having full-length mRNA that encodes for one or clotting enzymes, such as Factor VIII, Factor IX, Factor VII, and Factor X.

g. Kidney

In some embodiments, the present disclosure provides a formulation having full-length mRNA that encodes a peptide or polypeptide for use in the delivery to or treatment of the kidney of a subject or a kidney cell. In some embodiments, the present disclosure provides a formulation having full-length mRNA that encodes for collagen type IV alpha 5 chain (COL4A5) protein.

h. Eye

In some embodiments, the present disclosure provides a formulation having full-length mRNA that encodes a peptide or polypeptide for use in the delivery to or treatment of the eye of a subject or an eye cell. In some embodiments, the present disclosure provides a formulation having full-length mRNA that encodes for ATP-binding cassette sub-family A member 4 (ABCA4) protein. In some embodiments, the present disclosure provides a formulation having full-length mRNA that encodes for retinoschisin protein. In some embodiments, the present disclosure provides a formulation having full-length mRNA that encodes for retinal pigment epithelium-specific 65 kDa (RPE65) protein. In some embodiments, the present disclosure provides a formulation having full-length mRNA that encodes for centrosomal protein of 290 kDa (CEP290).

### Proteins for Vaccine

In some embodiments, a formulation described herein comprises a full-length mRNA that encodes for a peptide or polypeptide for use in the delivery of or treatment with a vaccine for a subject or a cell of a subject suitable for the present disclosure. Additional embodiments are described herein.

In some embodiments, a formulation described herein comprises a full-length mRNA that encodes for an antigen (e.g., from an infectious agent such as a virus) suitable for the present disclosure. In some embodiments, a formulation described herein comprises a full-length mRNA that encodes for an immunomodulator suitable for the present disclosure. In some embodiments, a formulation described herein comprises a full-length mRNA that encodes for an endonuclease suitable for the present disclosure.

a. Exemplary Proteins for Cancer Vaccines

Cancer is considered an immunological disease, and cancer immunotherapy has become the center-stage of research and development of the present day. In cancer immunotherapy, vaccines are developed to boost the immune system to turn against cancer antigens and eliminate a tumor by activated cytotoxic T cells directed against the antigens. One class of cancer antigens are viral antigens relating to the cancer-causing viruses, for example, Epstein Barr Virus (EBV) antigens such as EBV1 and EBV2 associated with lymphoma and nasopharyngeal carcinoma, Human Papilloma Virus (HPV) antigens such as HPV16 associated with cervical cancer (CC), Hepatitis B Virus (HBV) antigens and Hepatitis C Virus (HCV) antigens associated with hepatocellular carcinoma (HCC), human T lymphotropic virus type 1 (HTLV-1) associated with adult T-cell leukemia/lymphoma, and human herpes virus 8 (HHV-8) with Kaposi sarcoma, to name a few.

On the other hand, cancerous cells express antigens that are not commonly expressed by non-cancerous cells or tissues. Such antigens include but are not limited to epithelial tumor antigen (ETA) found in breast cancer, RAS family member p-53 and other activated RAS antigens, ovarian cancer antigens BRCA1 and BRCA2, melanoma associated antigen (MAGE) found in malignant melanoma cells, BCR-ABL fusion gene product found in myeloid leukemia, acute lymphoblastic leukemia, acute myelogenic leukemia, BRAF antigens found in cutaneous melanoma and colorectal cancer, epithelial growth factor receptor (EGFR) for non-small cell lung cancer, KRAS found in colorectal and non-small cell lung cancer, Neuron specific enolase, found in neuroblastoma and non-small cell lung cancer, NY-ESO 1 found in neuroblastoma, Melanoma-associated antigen recognized by T cells (MART-1) found in melanoma, programmed death ligand 1 (PD-L¹) found in non-small cell lung cancer, Prostate-specific antigen (PSA) found in prostate cancer, urokinase plasminogen activator (UPA), plasminogen activator inhibitor (PAI-1) found in breast cancer, and many others, almost all of which are mutated endogenous proteins. These cancer antigens, being endogenous are not presented by antigen presenting cells (APCs) in a manner similar to viral antigens, i.e., in association with the MHC-1 molecule categorizing the antigen as foreign, but cytotoxic T cells are able to differentiate and identify mutated self-antigens and possess an inherent property to seek and destroy cells that bear the mutated antigens. Therefore, the current objective of cancer immunotherapy is to achieve optimum activation of cytotoxic T cells directed against the mutated antigens. A patient's specific mutations associated with her/his cancer can be mapped and used to generate vaccines, inducing the patient's own cytotoxic T cells to generate the necessary immune response to destroy tumor cells. mRNA vaccines could be the safe and cost-effective alternative to peptide vaccines for enabling such personalized medicine. Pan-genomic scanning and analysis of mutations present in a cancer patient could be used to specifically design mRNA encoding an antigen or epitope containing the mutation, which when administered in vivo will produce the translated product on a cell surface. This would direct an immune response against the mutated antigen. In the process, cytotoxic T cells attack the tumor cells, which inherently express the mutated antigen. Methods and protocols involved in executing pan genomic sequencing analysis, mutation analysis, epitope mapping and analysis and designing suitable peptides for vaccination are known to one of skill in the art.

This approach could also be leveraged to find dominant and subdominant antigens in a patient. It has been observed that both in chronic infection and cancer, certain antigens play a dominant role in producing an initial immune response. But soon afterwards, tolerance against such dominant antigens sets in, thereby the immune response is dampened. Genomic analysis and identification of antigens which did not show an initial dominant antigenic response (often termed subdominant antigens) could now be used to generate new and revived immune response.

One advantage of mRNA vaccines over peptide vaccines is that mRNA vaccines bypass the HLA-matching for the receiving host.

In a synthetic approach to mRNA vaccine design, a pathogen proteome can be scanned for antigenic signatures with vaccine potential. (Proteome database can be accessed using Uniprot Consortium, uniprot.org/). This could be effective in new pathogens, such as Zika virus. This type of reverse vaccinology has been employed in identifying a number of novel peptide vaccine candidates. New peptide vaccines were also identified from Helicobacter pylori and Mycobacterium tuberculosis by combining genomics and proteomics (See, for example, Etz et al., PNAS. 2002, 99 (10) 6573-6578). Whether the potential antigenic candidate can generate successful immune response can be verified by suitably expressing a library of potential antigens by various forms of cell surface display and subjecting to testing opsonization and antibody binding. Exemplary useful databases for vaccine antigen development include: ImMunoGeneTics information system (URL: imgt.org); Epitome Database, (URL: rostlab.org/services/epitome), Immune Epitope Database and Analysis Resource, iedb.org; Immunet database, immunet.cn/ced/index.php; HIV database: hiv.lanl.gov/content/immunology for immunogenetics and immunoinformatics.

Therefore, from the above discussion, it is clear that enrichment of mRNA vaccine delivery to lymph nodes can lead to access of the vaccines to both activated and naïve lymphocytes for lymphoproliferation and antigen-specific T cell and B cell generation. This localization of antigen activation is also less toxic as opposed to widespread immune activation.

### b Exemplary Proteins for Vaccines Against Infectious Diseases

mRNA vaccines offer many advantages over present cell-based vaccines using live, attenuated or killed pathogen or toxoid vaccines. The dry powder formulation of the present disclosure can used to deliver mRNA for vaccination. In addition to the safety, mRNA vaccines are cost-effective and provide flexible design platform. mRNA encoding an antigen could be directed to induce specific immune response, and therefore can be applied in developing a wide range of therapeutic and prophylactic mRNA vaccines for a wide variety of diseases, including infections and cancers.

Recent outbreaks of emerging and re-emerging infectious diseases worldwide, such as COVID-19, severe acute respiratory syndrome (SARS), Middle East respiratory syndrome (MERS), measles, avian, pandemic influenza, chikungunya virus, Ebola virus disease (EVD), Zika virus disease, have resulted in a renewed focus on infectious diseases. There is a need for constant readiness and preparedness to deal with infectious disease outbreaks including emerging and re-emerging infectious disease threats.

Vaccine candidates are well established for a large number of infectious pathogens. Typically, vaccines are agents that mimic at least in part a disease-causing agent and thereby elicit an immune response by the mammalian host. In general vaccines are biological agents, such as heat killed, irradiated or otherwise attenuated pathogenic organisms, live attenuated microbes, protein or peptide antigens, conjugated antigens, toxins or microbial surface proteins or fragments thereof. However, an mRNA encoding a protein or a peptide antigen, is a safe and effective way to induce an immune response against the disease. As discussed above, mRNA can be effectively delivered to express in vivo by encapsulated in a liposome comprising suitable lipids discussed in a later section. This mRNA encoding the antigenic peptide or protein could therefore be used to generate the vaccine in vivo. An immune response generated by the mammalian host against the vaccine component is intended in turn to protect the host from a subsequent attack by the pathogen, since the immune system of the host is primed for the attack by the pathogen. In other words, the host system has immunological memory (a component of the adaptive immune response) of the pathogen. This process is known as prophylactic vaccination. Additionally, a vaccine may boost the host's immune system in an existing infection, for example by redirect an immune response against new and less recognized microbial antigen(s) (subdominant antigens) which then induce a strong immune response leading to pathogen elimination. This type of vaccine response may be categorized as therapeutic vaccination.

An immune response against a pathogen can be broken down into a few stages. First, an encounter of the human body (or a mammalian system) with a new pathogen, especially by contact through exposed surfaces such as skin or the internal mucosal surfaces of the respiratory, gastro-intestinal, and urogenital tracts, lead to a non-specific innate immune response through activation of pattern recognition molecules. Pattern recognition molecules include a variety of germline-encoded receptors specialized in discriminating between microbial and host cell surfaces, or infected and normal cells. Phagocytes (monocytes, macrophages and dendritic cells), express pattern recognition molecules on their surface and are primarily responsible for recognizing, killing and elimination of the pathogens in an innate immune response. In doing so, phagocytes also process and present antigens to the circulating lymphocytes for generating a more specific antigen-targeted immune response, also known as the adaptive immune response. At this stage activated lymphocytes mature in the lymph nodes into antigen-specific T-cells expressing receptors for recognition of the antigen such that effector cytotoxic T cells recognize and kill a cell expressing the antigen when present in association with a second set of cell-surface molecules, the Major Histocompatibility Complex molecules or MHC; and helper T cells activate the system to generate the T cell memory and the humoral immune response. The humoral immune response comprises antibody-secreting B cells generated by clonal expression and differentiation over the course of several days, during which time that innate immunity continues to function. Clonal expansion of cytotoxic T cells also occurs rapidly in lymphoid organs, such as lymph nodes and is augmented by exposure to antigens. Activated T cells generate a number of cytokines, such as Interferon Gamma (IFN—Y) and Tumor Necorsis Factor alpha (TNF-α) which are considered the hallmarks of T cell activation. Eventually, antigen-specific T cells and then antibodies are released into the blood and recruited to the site of infection. A successful vaccine generates a rapid and robust cytotoxic T cell response, a strong antibody response and a lasting immunological memory.

### Exemplary Cellular Maintenance Proteins

In some embodiments, the present disclosure provides a formulation having full-length mRNA that encodes for a protein associated with a lipid metabolism or fibrotic disorder. In some embodiments, the present disclosure provides a formulation having full-length mRNA that encodes for an mTOR inhibitor. In some embodiments, the present disclosure provides a formulation having full-length mRNA that encodes for ATPase phospholipid transporting 8B1 (ATP8B1) protein. In some embodiments, the present disclosure provides a formulation having full-length mRNA that encodes for one or more NF-kappa B inhibitors, such as one or more of I-kappa B alpha, interferon-related development regulator 1 (IFRD1), and Sirtuin 1 (SIRT1). In some embodiments, the present disclosure provides a formulation having full-length mRNA that encodes for PPAR-gamma protein or an active variant.

In some embodiments, the present disclosure provides a formulation having full-length mRNA that encodes a peptide or polypeptide for use in the delivery of or treatment with a vaccine for a subject or a cell of a subject. For example, in some embodiments, the present disclosure provides a formulation having full-length mRNA that encodes for an antigen from an infectious agent, such as a virus. In some embodiments, the present disclosure provides a formulation having full-length mRNA that encodes for an antigen from influenza virus. In some embodiments, the present disclosure provides a formulation having full-length mRNA that encodes for an antigen from respiratory syncytial virus. In some embodiments, the present disclosure provides a formulation having full-length mRNA that encodes for an antigen from rabies virus. In some embodiments, the present disclosure provides a formulation having full-length mRNA that encodes for an antigen from cytomegalovirus. In some embodiments, the present disclosure provides a formulation having full-length mRNA that encodes for an antigen from rotavirus. In some embodiments, the present disclosure provides a formulation having full-length mRNA that encodes for an antigen from a hepatitis virus, such as hepatitis A virus, hepatitis B virus, or hepatitis C virus. In some embodiments, the present disclosure provides a formulation having full-length mRNA that encodes for an antigen from human papillomavirus. In some embodiments, the present disclosure provides a formulation having full-length mRNA that encodes for an antigen from a herpes simplex virus, such as herpes simplex virus 1 or herpes simplex virus 2. In some embodiments, the present disclosure provides a formulation having full-length mRNA that encodes for an antigen from a human immunodeficiency virus, such as human immunodeficiency virus type 1 or human immunodeficiency virus type 2. In some embodiments, the present disclosure provides a formulation having full-length mRNA that encodes for an antigen from a human metapneumovirus. In some embodiments, the present disclosure provides a formulation having full-length mRNA that encodes for an antigen from a human parainfluenza virus, such as human parainfluenza virus type 1, human parainfluenza virus type 2, or human parainfluenza virus type 3. In some embodiments, the present disclosure provides a formulation having full-length mRNA that encodes for an antigen from malaria virus. In some embodiments, the present disclosure provides a formulation having full-length mRNA that encodes for an antigen from zika virus. In some embodiments, the present disclosure provides a formulation having full-length mRNA that encodes for an antigen from chikungunya virus.

In some embodiments, the present disclosure provides a formulation having full-length mRNA that encodes for an antigen associated with a cancer of a subject or identified from a cancer cell of a subject. In some embodiments, the present disclosure provides a formulation having full-length mRNA that encodes for an antigen determined from a subject's own cancer cell, i.e., to provide a personalized cancer vaccine.

In some embodiments, the present disclosure provides a formulation having full-length mRNA that encodes for an antibody. In some embodiments, the antibody can be a bi-specific antibody. In some embodiments, the antibody can be part of a fusion protein. In some embodiments, the present disclosure provides a formulation having full-length mRNA that encodes for an antibody to OX40. In some embodiments, the present disclosure provides a formulation having full-length mRNA that encodes for an antibody to VEGF. In some embodiments, the present disclosure provides a formulation having full-length mRNA that encodes for an antibody to tissue necrosis factor alpha. In some embodiments, the present disclosure provides a formulation having full-length mRNA that encodes for an antibody to CD3. In some embodiments, the present disclosure provides a formulation having full-length mRNA that encodes for an antibody to CD19.

In some embodiments, the present disclosure provides a composition having full-length mRNA that encodes for an immunomodulator. In some embodiments, the present disclosure provides a formulation having full-length mRNA that encodes for Interleukin 12. In some embodiments, the present disclosure provides a composition having full-length mRNA that encodes for Interleukin 23. In some embodiments, the present disclosure provides a formulation having full-length mRNA that encodes for Interleukin 36 gamma. In some embodiments, the present disclosure provides a formulation having full-length mRNA that encodes for a constitutively active variant of one or more stimulator of interferon genes (STING) proteins.

### Polypeptides Involved in CRISPR/Cas9 System:

In some embodiments, the present disclosure provides a composition having full-length mRNA that encodes for an endonuclease. In some embodiments, the present disclosure provides a formulation having full-length mRNA that encodes for an RNA-guided DNA endonuclease protein, such as Cas 9 protein. In some embodiments the present disclosure provides a formulation having full-length mRNA that encodes for a meganuclease protein. In some embodiments, the present disclosure provides a formulation having full-length mRNA that encodes for a transcription activator-like effector nuclease protein. In some embodiments, the present disclosure provides a formulation having full-length mRNA that encodes for a zinc finger nuclease protein.

Exemplary Proteins found in Various Cellular Organelles

In some embodiments, the present disclosure provides a formulation comprising full-length mRNA that encodes a secreted protein. In some embodiments, the present disclosure provides a formulation comprising full-length mRNA that encodes a nuclear protein. In some embodiments, the present disclosure provides a formulation comprising full-length mRNA that encodes a metabolic protein. In some embodiments, the present disclosure provides a formulation comprising full-length mRNA that encodes a cytoplasmic protein. In some embodiments, the present disclosure provides a composition comprising full-length mRNA that encodes a membrane protein. In some embodiments, the present disclosure provides a formulation comprising full-length mRNA that encodes a mitochondrial protein. In some embodiments, the present disclosure provides a formulation comprising full-length mRNA that encodes a lysosomal protein. In some embodiments, an mRNA encodes a cytosolic protein. In some embodiments, an mRNA encodes a protein associated with the actin cytoskeleton. In some embodiments, an mRNA encodes a protein associated with the plasma membrane.

### Exemplary Metabolic and Catabolic Proteins

In some embodiments, the present disclosure provides a formulation having full-length mRNA that encodes a peptide or polypeptide for use in the delivery to or treatment of the liver of a subject or a liver cell. Such peptides and polypeptides can include those associated with a urea cycle disorder, associated with a lysosomal storage disorder, with a glycogen storage disorder, associated with an amino acid metabolism disorder, associated with a lipid metabolism or fibrotic disorder, associated with methylmalonic acidemia, or associated with any other metabolic disorder for which delivery to or treatment of the liver or a liver cell with enriched full-length mRNA provides benefit.

In some embodiments, the present disclosure provides a formulation having full-length mRNA that encodes for a protein associated with a urea cycle disorder. In some embodiments, the present disclosure provides a formulation having full-length mRNA that encodes for ornithine transcarbamylase (OTC) protein. In some embodiments, the present disclosure provides a formulation having full-length mRNA that encodes for arginosuccinate synthetase 1 protein. In some embodiments, the present disclosure provides a formulation having full-length mRNA that encodes for carbamoyl phosphate synthetase 1 protein. In some embodiments, the present disclosure provides a formulation having full-length mRNA that encodes for arginosuccinate lyase protein. In some embodiments, the present disclosure provides a formulation having full-length mRNA that encodes for arginase protein.

In some embodiments, the present disclosure provides a formulation having full-length mRNA that encodes for a protein associated with methylmalonic acidemia. For example, in some embodiments, the present disclosure provides a formulation having full-length mRNA that encodes for methylmalonyl CoA mutase protein. In some embodiments, the present disclosure provides a formulation having full-length mRNA that encodes for methylmalonyl CoA epimerase protein.

In some embodiments, the present disclosure provides a formulation having full-length mRNA that encodes for a protein associated with a lysosomal storage disorder. In some embodiments, the present disclosure provides a formulation having full-length mRNA that encodes for alpha galactosidase protein. In some embodiments, the present disclosure provides a formulation having full-length mRNA that encodes for glucocerebrosidase protein. In some embodiments, the present disclosure provides a formulation having full-length mRNA that encodes for iduronate-2-sulfatase protein. In some embodiments, the present disclosure provides a formulation having full-length mRNA that encodes for iduronidase protein. In some embodiments, the present disclosure provides a therapeutic formulation having full-length mRNA that encodes for N-acetyl-alpha-D-glucosaminidase protein. In some embodiments, the present disclosure provides a formulation having full-length mRNA that encodes for heparan N-sulfatase protein. In some embodiments, the present disclosure provides a formulation having full-length mRNA that encodes for galactosamine-6 sulfatase protein. In some embodiments, the present disclosure provides a formulation having full-length mRNA that encodes for beta-galactosidase protein. In some embodiments, the present disclosure provides a formulation having full-length mRNA that encodes for lysosomal lipase protein. In some embodiments, the present disclosure provides a formulation having full-length mRNA that encodes for arylsulfatase B (N-acetylgalactosamine-4-sulfatase) protein. In some embodiments, the present disclosure provides a formulation having full-length mRNA that encodes for transcription factor EB (TFEB).

In some embodiments, the present disclosure provides a formulation having full-length mRNA that encodes for a protein associated with a glycogen storage disorder. In some embodiments, the present disclosure provides a formulation having full-length mRNA that encodes for acid alpha-glucosidase protein. In some embodiments, the present disclosure provides a formulation having full-length mRNA that encodes for glucose-6-phosphatase (G6PC) protein. In some embodiments, the present disclosure provides a formulation having full-length mRNA that encodes for liver glycogen phosphorylase protein. In some embodiments, the present disclosure provides a formulation having full-length mRNA that encodes for muscle phosphoglycerate mutase protein. In some embodiments, the present disclosure provides a formulation having full-length mRNA that encodes for glycogen debranching enzyme.

In some embodiments, the present disclosure provides a formulation having full-length mRNA that encodes for a protein associated with amino acid metabolism. In some embodiments, the present disclosure provides a formulation having full-length mRNA that encodes for phenylalanine hydroxylase enzyme. In some embodiments, the present disclosure provides a formulation having full-length mRNA that encodes for glutaryl-CoA dehydrogenase enzyme. In some embodiments, the present disclosure provides a formulation having full-length mRNA that encodes for propionyl-CoA carboxylase enzyme. In some embodiments the present disclosure provides a formulation having full-length mRNA that encodes for oxalase alanine-glyoxylate aminotransferase enzyme.

In some embodiments, the present disclosure provides a formulation comprising a nucleic acid, including, for example, a therapeutic nucleic acid, including, but not limited to a plasmid, a viral vector, an antisense nucleic acid, an siRNA, microRNA, ribozymes, antagomirs, aptamers, CRISPR nucleic acids (e.g., guide RNA, crRNA, or tracr RNA), nucleic acids for gene therapy, nucleic acids for DNA editing, probes, or any other oligonucleotide that is susceptible to degradation by nucleases and/or harsh environmental conditions (e.g., pH), including other oligonucleotides that are to be administered in vivo.

### Methods of Delivery

Compositions described herein can be administered according to various methods known in the art, whether as a dry powder formulation or following reconstitution. For example, after reconstitution, the pharmaceutical formulations of the disclosure may be administered in a local rather than systemic manner, for example, via injection of the pharmaceutical formulation directly into a targeted tissue, including sustained release formulations.

For example, dry powder formulations and/or reconstituted dry powder formulations of the present invention may be administered and dosed in accordance with current medical practice, taking into account the clinical condition of the subject, the nature of the encapsulated materials, the site and method of administration, the scheduling of administration, the subject's age, sex, body weight and other factors relevant to clinicians of ordinary skill in the art.

Suitable routes of dry powder formulations and/or reconstituted dry powder formulations disclosed herein include, for example, oral, rectal, vaginal, transmucosal, or intestinal administration; parenteral delivery, including intramuscular, subcutaneous, intramedullary injections, as well as intrathecal, intracerebroventricular. direct intraventricular, intravenous, intraperitoneal, intranasal, or intraocular injections or infusions.

Dry powder formulations and/or reconstituted dry powder formulations of the present invention may be administered in a local rather than systemic manner. For example, a reconstituted dry powder formulation may be administered via injection or infusion of the pharmaceutical compositions directly into a targeted tissue, preferably in a depot or sustained release formulation, such that the contacting of the targeted cells with the constituent lipid nanoparticles may further facilitated. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations of the present invention complexed with therapeutic molecules or ligands can even be surgically administered, for example in association with a polymer or other structure or substance that can allow the compositions to diffuse from the site of implantation to surrounding cells. Alternatively, such compositions can be applied surgically without the use of polymers or supports.

Still further exemplary, non-limiting modes of delivery are described herein.

i. Targetted Delivery

Local delivery can be affected in various ways, depending on the tissue to be targeted. Exemplary tissues in which delivered mRNA may be delivered and/or expressed include, but are not limited to the lungs, liver, kidney, heart, spleen, serum, brain, skeletal muscle, lymph nodes, skin, and/or cerebrospinal fluid. In some embodiments, the tissue to be targeted in the liver. For example, aerosols containing compositions of the present disclosure can be inhaled (for nasal, tracheal, or bronchial delivery).

ii. Delivery to the Airways

In some embodiments, compositions of the present disclosure can be delivered using a metered dose inhaler. In some embodiments, compositions of the present disclosure can be reconstituted and nebulized for delivery. In some embodiments, compositions of the present disclosure can be injected into the site of injury, disease manifestation, or pain. In some embodiments, compositions of the present disclosure can be provided for oral, tracheal, or esophageal applications.

In some embodiments, a formulation of the present disclosure is reconstituted into a liquid solution and nebulized for delivery. Nebulization can be achieved by any nebulizer known in the art. A nebulizer transforms a liquid to a mist so that it can be inhaled more easily into the lungs. Nebulizers are effective for infants, children and adults. Nebulizers are able to nebulize large doses of inhaled medications. Typically, a nebulizer for use with the disclosure comprises a mouthpiece that is detachable.

Therapeutic treatments for liver and lung diseases are being developed by delivering synthetic mRNA encoding a missing and/or a non-functional protein to the corresponding organ cells via intravenous and inhalable routes, respectively.

For pulmonary delivery in particular, the particles of the formulation affect distribution and deposition of an aerosol within the respiratory system. In many cases, particle deposition to the large conducting airways is preferred for effective absorption and distribution of the therapeutic component. Aerosol of very fine particles, for instance, particles having less than 1 micrometer diameter may be deposited peripherally for effective absorption by specific cells of the lung, such as smooth muscles for an active pharmaceutical ingredient functioning as bronchodilator. In some embodiments, formulations comprising trehalose are used for lung delivery. In some embodiments, the formulations comprising a sugar or sugar alcohol excipients (for example, trehalose) having a particle size less than 5 microns are suited to lung delivery.

iii. Tablets and Granules

In some embodiments, compositions of the present disclosure can be supplied in reconstituted liquid, powder, tablet, granules, implants, or capsule form for administration to the stomach or intestines. In some embodiments, the pharmaceutical composition is in the form of a tablet with an enteric coating.

In some embodiments, the enteric coating comprises: hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, cellulose ester-ether phthalate, hydroxypropylcellulose phthalate, alkali salts of cellulose acetate phthalate, alkaline earth salts of cellulose acetate phthalate, hydroxypropylmethyl cellulose phthalate, cellulose acetate phthalate, carboxymethylcellulose sodium, acrylic acid polymers and copolymers, ethyl acrylate/methyl methacrylate/ethyl trimethylammonium chloride methacrylate terpolymer, methacrylic acid/ethyl acrylate copolymer, methacrylic acid/methyl methacrylate copolymer, polyvinyl pyrrolidone, polyvinyl acetate, polyvinylacetate phthalate, vinylacetate crotonic acid copolymer; shellac, ammoniated shellac, shellac-acetyl alcohol, or shellac n-butyl stearate.

In some embodiments, the tablets are manufactured using filler-binders for direct compaction and to promote cohesiveness. Some exemplary binders include Polyvinyl Pyrrolidone (PVP), Methylcellulose, Hydroxy Propyl Methyl Cellulose (HPMC), Polymethacrylates, Sodium Carboxy Methyl Cellulose, Polyethylene Glycol (PEG) and Methylcellulose, Sucrose, Acacia, Methyl Cellulose, Liquid glucose, Tragacanth, Ethyl Cellulose, Gelatin, Starch Paste, Hydroxy Propyl Cellulose, Pregelatinized Starch, Sodium Carboxy Methyl Cellulose, Alginic Acid, Polyvinyl Alcohols, Polymethacrylates.

iv. Other Routes of Administration

In some embodiments, compositions of the present disclosure can be supplied for rectal or vaginal application. In some embodiments, compositions of the present disclosure can be delivered to the eye as drops, or even intravitreal or intraocular injection.

In some embodiments, compositions of the present disclosure can be used for any parenteral and mucosal routes. In some embodiments liquid formulation can be administered by for example, by infusion, perfusion, administration using a pen injector, cartridge system needle-array or patch and/or administration by a catheter system. In some embodiments the administration is by subcutaneous injection, intradermal injection, subdermal injection, intramuscular injection, or topical administration. There are many common forms of topical medication such as lotions, gels, patches, and powders, creams and ointments. In some embodiments, the parenteral administration includes buccal, sublingual, palatal, gingival, nasal, vaginal, cervical, rectal, or transdermal administration. In some embodiments, parenteral administration includes, e.g., intravenous, intramuscular, intra-arteriole, intradermal, subcutaneous, intraperitoneal, and intraventricular.

In some embodiments, methods of administration may further include oral administration. In some examples, administration orally may include a mouthwash, a mouth rinse, an oral rinse, a mouth bath, and the like. For examples, cervical and/or vaginal mucosa administration may be via a solution, a gel, a suspension, a cream, an ointment, a foam, a pessary, or a tablet. In one aspect, the reconstituted composition may be administered to the cervical and/or vaginal mucosa of a subject. The cervical and/or vaginal mucosa administration may be via a solution, gel, suspension, cream, ointment, foam, pessary, or tablet.

In some embodiments, the formulation or the reconstituted formulation is suitable for mucosal delivery. In some embodiments, the formulation is suitable for oral delivery. In some embodiments, the formulation is suitable for sublingual delivery. In some embodiments, the formulation is suitable for or intranasal delivery. In some embodiments, the formulation is suitable for buccal delivery. In some embodiments, the formulation is suitable for intramuscular. In some embodiments, the formulation is suitable for intravenous. In some embodiments, the formulation is suitable for subcutaneous.

### First Representative Embodiments of the Present Disclosure

1. A dry powder formulation for reconstitution comprising
   (a) an excipient that is sucrose or trehalose; and
   (b) a lipid nanoparticle (LNP), comprising messenger RNA (mRNA) encapsulated by one or more lipids; and wherein the w/w ratio of the excipient of (a) and the total lipids in the LNP of (b) is at least about 5.
2. The dry powder formulation of numbered embodiment 1, wherein the excipient is sucrose.
3. The dry powder formulation of numbered embodiment 1, wherein the excipient is trehalose.
4. The dry powder formulation of any one of numbered embodiments 1-3, wherein the w/w ratio of the excipient to the total lipids is at least about 5.6, 11, or 15.
5. The dry powder formulation of numbered embodiment 4, wherein the w/w ratio of the excipient to the total lipids is at least about 11.1 or at least about 15.6.
6. The dry powder formulation of any one of numbered embodiments 1-5, comprising an LNP that comprises one or more cationic lipids, one or more non-cationic lipids, and one or more PEG-modified lipids.
7. A dry powder formulation for reconstitution comprising
   (a) an excipient that is sugar or sugar alcohol; and
   (b) a lipid nanoparticle (LNP), comprising messenger RNA (mRNA) encapsulated by one or more lipids; and.
wherein the w/w ratio of the excipient of (a) and the total lipids in the LNP of (b) is at least about 5.
8. The dry powder formulation of numbered embodiment 7, wherein the sugar or the sugar alcohol is selected from sucrose, mannitol, xylitol, lactose, and trehalose.
9. The dry powder formulation of numbered embodiment 7, wherein the sugar or the sugar alcohol is trehalose.
10. The dry powder formulation of numbered embodiment 7, wherein the sugar or the sugar alcohol is sucrose.
11. The dry powder formulation of any one of numbered embodiments 7-10, wherein the w/w ratio of the sugar or the sugar alcohol to the total lipids is at least about 5.6, 11, or 15.
12. The dry powder formulation of numbered embodiment 7, wherein the w/w ratio of the sugar or the sugar alcohol to the total lipids is at least about 11.1 or at least about 15.6.
13. The dry powder formulation of any one of numbered embodiments 7-12, comprising an LNP that comprises one or more cationic lipids, one or more non-cationic lipids, and one or more PEG-modified lipids.
14. The dry powder formulation of any one of numbered embodiments 1-13, wherein the particle size of the reconstituted dry powder formulation is less than 120 nm.
15. The dry powder formulation of any one of numbered embodiments 1-14, wherein the encapsulation rate is greater than 60%.
16. The dry powder formulation of any one of numbered embodiments 1-15, wherein the particle size of the dry powder is less than 5 μm.
17. The dry powder formulation of any one of numbered embodiments 1-16, wherein the formulation is stable after prolonged storage.
18. The dry powder formulation of numbered embodiment 17, wherein the mRNA maintains an integrity of 80% or greater after storage at 4° C. for at least six months.
19. The dry powder formulation of numbered embodiment 17, wherein the mRNA maintains an integrity of 80% or greater for up to 1 year.
20. The dry powder formulation of numbered embodiment 17, wherein the mRNA maintains an integrity of 80% or greater for at least 1 year.
21. The dry powder formulation of any one of numbered embodiments 1-20, wherein the mRNA encodes a therapeutic protein.
22. The dry powder formulation of any one of numbered embodiments 1-21, wherein the mRNA encodes an antigen.
23. The dry powder formulation of any one of numbered embodiments 1-22, wherein the formulation is suitable for a vaccine.
24. The dry powder formulation of any one of numbered embodiments 1-23, wherein the formulation is suitable for parenteral delivery.
25. The reconstituted dry powder formulation of numbered embodiment 24, wherein the formulation is suitable for intramuscular, intravenous, or subcutaneous delivery.
26. The reconstituted dry powder formulation of any one of numbered embodiments 1-25, wherein the formulation is suitable for mucosal delivery.
27. The reconstituted dry powder formulation of numbered embodiment 26, wherein the formulation is suitable for oral, sublingual, or intranasal delivery.
28. A method of delivering mRNA in vivo comprising administering to a subject in need of a reconstituted dry powder formulation of any one of numbered embodiments 1-27.
29. A method of treating a disease or disorder in a subject by administering to the subject a reconstituted dry powder formulation of any one of numbered embodiments 1-28.
30. The method of numbered embodiment 28 or 29, wherein the reconstituted dry powder formulation is administered subcutaneously, intravenously, or intramuscularly. 31. A method of preparing a dry powder formulation for reconstitution, the method comprising:
   (a) combining a mixture comprising lipid nanoparticles (LNPs) encapsulating an mRNA and an ethanolic solution and wherein the ethanolic solution comprises an excipient that is sucrose or trehalose at a concentration of about 1-11% w/v, and
   (b) spray-drying the mixture, thereby obtaining the dry powder formulation.
32. A method of preparing a reconstituted dry powder formulation, comprising
   (a) providing a dry powder formulation prepared according to numbered embodiment 31; and
   (b) reconstituting the dry powder formulation in water or buffer, thereby obtaining the reconstituted dry powder formulation.
33. The method of numbered embodiment 32, wherein the reconstituted LNPs have a diameter of about 100 nm.
34. The method of numbered embodiment 31 or 32, wherein the ratio of sucrose or trehalose to the total lipids (w/w) is at least about 5, at least about 11, or at least about 15.
35. The method of numbered embodiment 31 or 32, wherein the concentration of sucrose or trehalose is greater than 2% (w/v), greater than 5% (w/v), or greater than 7% (w/v) post-reconstitution.
36. The method of any one of numbered embodiments 31-35, wherein the concentration of the sugar is about 2 (w/v) to about 10% (w/v) post-reconstitution.
37. The method of any one of numbered embodiments 31-36, wherein the particle size of the reconstituted dry-powder formulation is less than 120 nm.
38. The method of any one of numbered embodiments 31-37, wherein the reconstituted LNP particle size is between 80-120 nm.
39. The method of any one of numbered embodiments 31-38, wherein the reconstituted LNP particle size is between 80-115 nm.

### Second Representative Embodiments of the Present Disclosure

1. A dry powder formulation for reconstitution comprising
   (a) an excipient that is sugar or sugar alcohol; and
   (b) a lipid nanoparticle (LNP), comprising messenger RNA (mRNA) encapsulated by one or more lipids; and.
   wherein the w/w ratio of the excipient of (a) and the total lipids in the LNP of (b) is at least about 5.
2. The dry powder formulation of embodiment 1, wherein the sugar or the sugar alcohol is
   (a) selected from sucrose, mannitol, xylitol, lactose, and trehalose; or
   (b) trehalose; or
   (c) sucrose.
3. The dry powder formulation of embodiment 1 or 2, wherein the w/w ratio of the sugar or the sugar alcohol to the total lipids is at least about 5.6, 11, or 15, optionally wherein the w/w ratio of the sugar or the sugar alcohol to the total lipids is at least about 11.1 or at least about 15.6.
4. The dry powder formulation of any one of embodiments 1-3, comprising an LNP that comprises one or more cationic lipids, one or more non-cationic lipids, and one or more PEG-modified lipids.
5. The dry powder formulation of any one of embodiments 1-4, wherein the particle size of the reconstituted dry powder formulation is less than 120 nm.
6. The dry powder formulation of any one of embodiments 1-5, wherein the encapsulation rate is greater than 60%.
7. The dry powder formulation of any one of embodiments 1-6, wherein the particle size of the dry powder is less than 5 μm.
8. The dry powder formulation of any one of embodiments 1-7, wherein the formulation is stable after prolonged storage, optionally wherein the mRNA maintains an integrity of
   (a) 80% or greater after storage at 4° C. for at least six months; or
   (b) 80% or greater for up to 1 year; or
   (c) 80% or greater for at least 1 year.
9. The dry powder formulation of any one of embodiments 1-8, wherein the mRNA encodes a therapeutic protein.
10. The dry powder formulation of any one of embodiments 1-9, wherein the mRNA encodes an antigen.
11. The dry powder formulation of any one of embodiments 1-10, wherein the formulation is suitable for a vaccine.
12. The dry powder formulation of any one of embodiments 1-11, wherein the formulation is suitable for
   (a) parenteral delivery, optionally wherein the formulation is suitable for intramuscular, intravenous, or subcutaneous delivery; and/or
   (b) mucosal delivery, optionally wherein the formulation is suitable for oral, sublingual, or intranasal delivery.
13. A method of delivering mRNA in vivo comprising administering to a subject in need of a reconstituted dry powder formulation of any one of embodiments 1-12, optionally wherein the reconstituted dry powder formulation is administered subcutaneously, intravenously, or intramuscularly.
14. A method of treating a disease or disorder in a subject by administering to the subject a reconstituted dry powder formulation of any one of embodiments 1-13, optionally wherein the reconstituted dry powder formulation is administered subcutaneously, intravenously, or intramuscularly.
15. A method of preparing a dry powder formulation for reconstitution, the method comprising:
   (a) combining a mixture comprising lipid nanoparticles (LNPs) encapsulating an mRNA and an ethanolic solution and wherein the ethanolic solution comprises an excipient that is sucrose or trehalose at a concentration of about 1-11% w/v, and
   (b) spray-drying the mixture, thereby obtaining the dry powder formulation, optionally
   (i) wherein the method further comprises
   (c) providing the dry powder formulation; and
   (d) reconstituting the dry powder formulation in water or buffer, thereby obtaining the reconstituted dry powder formulation, optionally, wherein the reconstituted LNPs have a diameter of about 100 nm; and/or
   (ii) wherein the ratio of sucrose or trehalose to the total lipids (w/w) is at least about 5, at least about 11, or at least about 15; or
   (iii) wherein the concentration of sucrose or trehalose is greater than 2% (w/v), greater than 5% (w/v), or greater than 7% (w/v) post-reconstitution; and/or
   (iv) wherein the concentration of the sugar is about 2 (w/v) to about 10% (w/v) post-reconstitution; and/or
   (v) wherein the particle size of the reconstituted dry-powder formulation is less than 120 nm; and/or
   (vi) wherein the reconstituted LNP particle size is between 80-120 nm; and/or
   (vii) wherein the reconstituted LNP particle size is between 80-115 nm.

### Examples

While certain compounds, compositions and methods of the present disclosure have been described with specificity in accordance with certain embodiments, the following examples serve only to illustrate the disclosure and are not intended to limit the same.

### Example 1—Preparation of mRNA-LNP Based DPP

The goal of this experiment was to synthesize mRNA-LNP based dry powder product (DPP) containing various sugar alcohol excipients.

Target mRNAs were synthesized by in vitro transcription employing RNA polymerase with a plasmid DNA template encoding the gene using unmodified nucleotides. This was followed by the addition of a 5′ cap structure (Cap 1) and a 3′ poly(A) tail. An ethanolic solution of a mixture of lipids (ionizable lipid, phosphatidylethanolamine, cholesterol and polyethylene glycol-lipid) were combined with an aqueous buffered solution of target mRNA at an acidic pH under controlled conditions to yield a suspension of uniform mRNA-LNPs. Upon ultrafiltration and diafiltration, mRNA-LNP suspensions were formulated into a final diluent containing different sugar-alcohol and sugar excipients at their desired concentration in 20% ethanol. The resultant mRNA-LNP suspension was spray dried on a Buchi-290 spray dryer using parameters as shown in Table 1. Varying excipient amounts as shown in Table 2 and Table 3 were evaluated to identify the optimum excipient to total lipid amount ratio to generate a stable reconstituted product.

Several mRNA-LNP containing dry powder products were generated with various sugar alcohol excipients.

TABLE 1
Spray drying conditions.

| | Inlet | | | Outlet |
|---|---|---|---|---|
| | Temperature | Aspirator | Pump | Temperature |
| Solvent | (° C.) | (%) | (%) | (° C.) |
| 20% Ethanol | 65 | 100 | 25 | 28-35 |

TABLE 2
Varying amounts of excipient (mannitol, xylitol, lactose, sucrose,
or trehalose) post-reconstituting the DPP at final mRNA and total
lipid concentrations of 0.2 and 4.5 mg/ml, respectively.

| | Total | | Excipient/ |
|---|---|---|---|
| mRNA | Lipid | Excipient | Total Lipid |
| (mg/ml) | (mg/ml) | (% w/v) | Ratio (w/w) |
| 0.2 | 4.5 | 7 | 15.6 |
| 0.2 | 4.5 | 5 | 11.1 |
| 0.2 | 4.5 | 2.5 | 5.6 |
| 0.2 | 4.5 | 1.25 | 2.8 |

TABLE 3
Varying amounts of excipient (mannitol, xylitol, lactose, sucrose,
or trehalose) in 20% ethanol before spray drying at final mRNA and
total lipid concentrations of 0.3 and 6.75 mg/ml, respectively.

| | Total | | Excipient/ |
|---|---|---|---|
| mRNA | Lipid | Excipient | Total Lipid |
| (mg/ml) | (mg/ml) | (% w/v) | Ratio (w/w) |
| 0.3 | 6.75 | 10.5 | 15.6 |
| 0.3 | 6.75 | 7.5 | 11.1 |
| 0.3 | 6.75 | 3.75 | 5.6 |
| 0.3 | 6.75 | 1.875 | 2.8 |

### Example 2-Effects of Various Sugars and Sugar Alcohol Excipients

This experiment was aimed at delineating the best sugar or sugar alcohol excipient for mRNA-LNP based DPP. The dry powder sprayability, as well as the physical properties such as, particle size, polydispersity index, were all analyzed and are summarized.

i. Xylitol and Lactose:

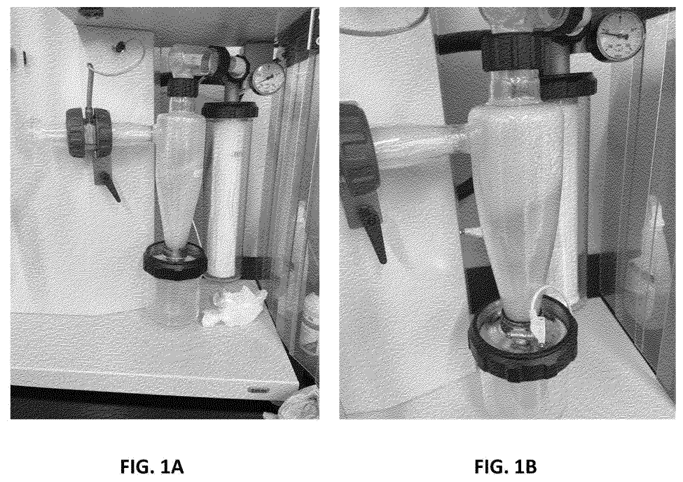

As shown in FIG. 1A and FIG. 1B, formulation with xylitol (FIG. 1A) and lactose (FIG. 1B) did not spray dry well even at highest excipient amount. The liquid formulation adhered to the cyclone separator and no dry powder product was collected in the collection vessel.

ii. Mannitol:

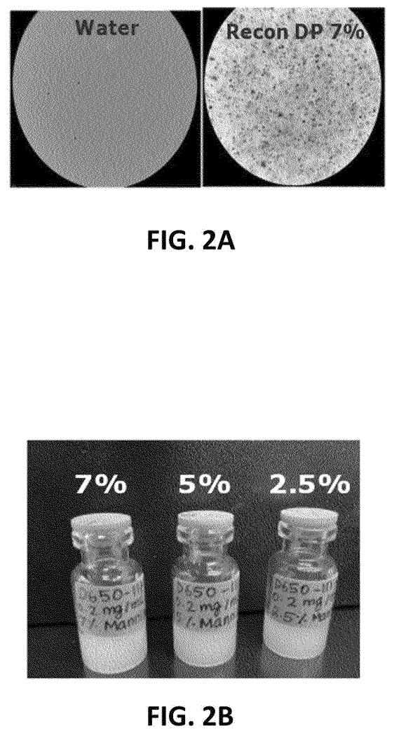

Although the dry powder characteristics were excellent, the DPP did not reconstitute. The yield of the DPP was about 35% at and above mannitol/lipid weight ratio of 5.6 and increased with the increasing mannitol/lipid ratio. Large aggregates were detected under 400× magnification in the reconstituted dry powder product despite using the highest mannitol/lipid weight ratio of 15.6 (i.e., 7% mannitol (w/v); FIG. 2A). Table 4 shows the dry powder characteristics and post reconstitution composition and characteristics with mannitol as the sugar excipient. FIG. 2A shows the aggregates observed in the reconstituted dry powder with 7% mannitol (w/v) as excipient under 400× magnification as compared to water alone. FIG. 2B shows the appearances of reconstituted dry powder with different amounts of mannitol as excipient (left: 7% mannitol (w/v); middle: 5% mannitol (w/v); right 2.5% mannitol (w/v)).

iii. Sucrose:

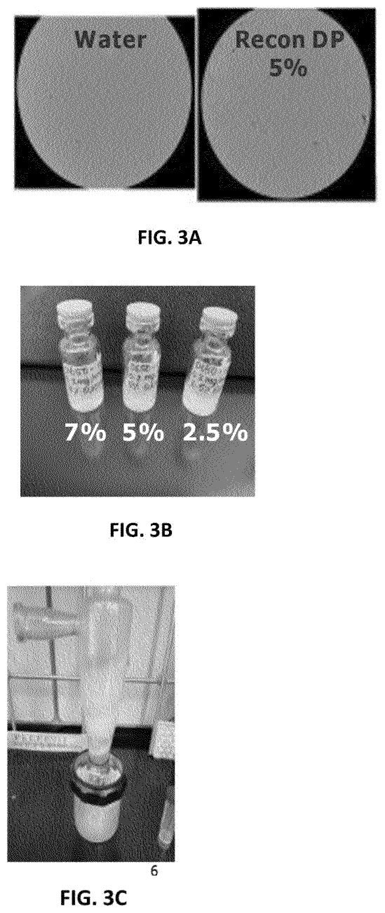

Although the DPP particle size was bigger, the product reconstituted well at and above sucrose to lipid ratio of 11.1. Although the DPP at sucrose/lipid ratio of 5.6 also reconstituted, the LNP particle size of the reconstituted product was larger (127 nm). The yield of the DPP was about 30% at and above sucrose/lipid weight ratio of 11.1. No aggregates were detected in the reconstituted product at and above sucrose/lipid weight ratio of 11.1 (FIG. 3A). Table 5 shows the dry powder characteristics and post reconstitution composition and characteristics with sucrose as the sugar excipient. FIG. 3A shows no aggregates were observed using a microscope for reconstituted dry powder with 5% sucrose (w/v) as excipient as compared to water alone. FIG. 3B shows the appearances of reconstituted dry powder with different amounts of sucrose as excipient (left: 7% sucrose (w/v); middle: 5% sucrose (w/v); right 2.5% sucrose (w/v)). FIG. 3C shows mRNA-LNP formulation with 2.5% sucrose (w/v) as excipient did not spray dry well and the liquid formulation adhered to the cyclone separator.

iv. Trehalose:

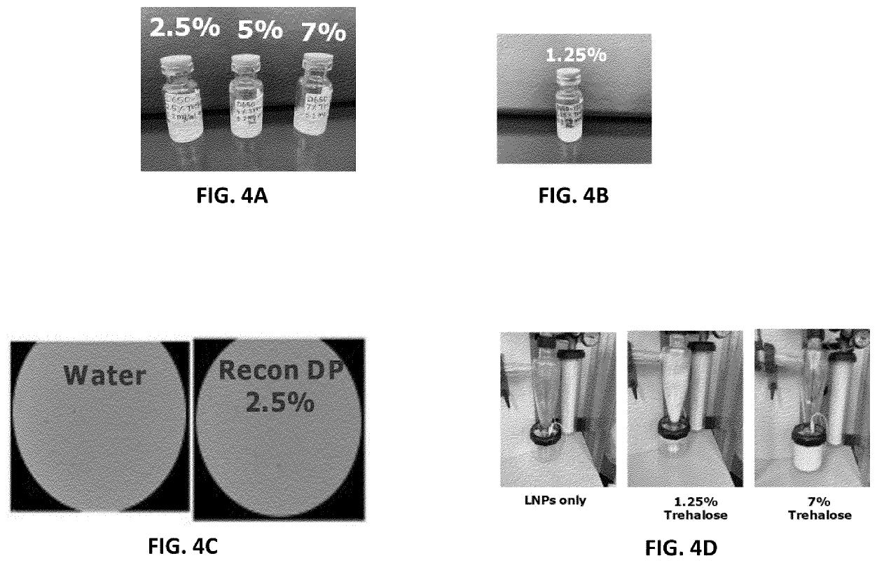

Not only was the dry powder particle size was under 5 micron for a trehalose based dry powder product but it also reconstituted well at and above trehalose/lipid weight ratio of 5.6. The yield of the DPP was excellent and above 75% at and above trehalose/lipid weight ratio of 5.6. The dry powder particle size was higher than 5 micron (81 μm) and the post reconstituted mRNA-LNP particle size was also higher (134 nm) for the dry powder prepared with a lower trehalose/lipid ratio of 2.8. No aggregates were detected in the reconstituted product at and above trehalose/lipid weight ratio of 5.6 as shown in the microscopic images (FIG. 4C). Table 6 shows the dry powder characteristics and post reconstitution composition and characteristics with trehalose as the sugar excipient. FIG. 4A shows the appearances of reconstituted dry powder with different amounts of trehalose as excipient (left: 2.5% trehalose (w/v); middle: 5% trehalose (w/v); right 7% trehalose (w/v)). FIG. 4B shows the appearance of reconstituted dry powder with 1.25% trehalose (w/v) as excipient. FIG. 4C shows no aggregates were observed using a microscope for reconstituted dry powder with 2.5% trehalose (w/v) as excipient as compared to water alone. FIG. 4D shows mRNA-LNP formulation with 1.25% trehalose (w/v) as excipient did not spray dry well with most of the liquid formulation adhered to the cyclone separator and only little dry powder collected in the collection vessel as compared to mRNA-LNP formulation with 7% trehalose (w/v) as excipient.

### V. Trehalose Added Externally as a Part of the Reconstituting Solvent:

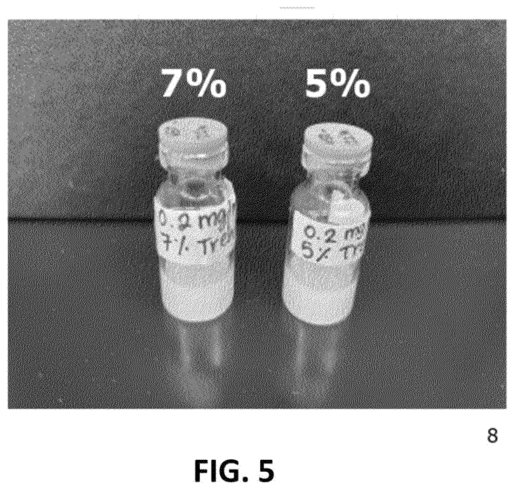

In order to prove that the excellent reconstitutable properties of the trehalose based DPP are associated with the presence of trehalose as a part of the DPP, trehalose was added externally in water to reconstitute the DPP manufactured using lower trehalose/lipid ratio of 2.8. As shown in Table 7 and FIG. 5, addition of trehalose externally did not improve reconstitution of the DPP and did not improve mRNA-LNP particle size post reconstitution. Table 7 shows the dry powder characteristics and post reconstitution composition and characteristics with a low amount of trehalose as the sugar excipient in the preparation of the DPP and reconstituted with different amounts of trehalose added externally as part of the reconstituting solvent. FIG. 5 shows the appearances of reconstituted dry powder with trehalose added as part of the reconstituting solvent at different concentrations.

TABLE 4
Dry powder characteristics and post reconstitution composition and
characteristics of mRNA-LNP dry powder with mannitol as excipient.

| Dry powder Characteristics | | | | Post Reconstitution Composition and Characteristics | | | | | | |
|---|---|---|---|---|---|---|---|---|---|---|
| | Size | Reconstitution | Reconstituted | mRNA | Lipid | Mannitol % | Mannitol/Lipid | Size | | Encapsulation |
| Yield % | (μm) | Solvent | (Yes/No) | (mg/ml) | (mg/ml) | (w/v) | ratio (w/w) | (nm) | PDI | (%) |
| 53 | 4.7 | Water | No | 0.2 | 4.5 | 7 | 15.6 | N/A | N/A | 78 |
| 45 | 4.5 | Water | No | 0.2 | 4.5 | 5 | 11.1 | N/A | N/A | 78 |
| 38 | 3.5 | Water | No | 0.2 | 4.5 | 2.5 | 5.6 | N/A | N/A | 87 |

TABLE 5
Dry powder characteristics and post reconstitution composition and
characteristics of mRNA-LNP dry powder with sucrose as excipient.

| Dry powder Characteristics | | | | Post Reconstitution Composition and Characteristics | | | | | | |
|---|---|---|---|---|---|---|---|---|---|---|
| | Size | Reconstitution | Reconstituted | mRNA | Lipid | Sucrose % | Sucrose/Lipid | Size | | Encapsulation |
| Yield % | (μm) | Solvent | (Yes/No) | (mg/ml) | (mg/ml) | (w/v) | ratio (w/w) | (nm) | PDI | (%) |
| 30 | 63.0 | Water | Yes | 0.2 | 4.5 | 7 | 15.6 | 102 | 0.188 | 83 |
| 33 | 170.0 | Water | Yes | 0.2 | 4.5 | 5 | 11.1 | 108 | 0.193 | 83 |
| 22 | 216.0 | Water | Yes | 0.2 | 4.5 | 2.5 | 5.6 | 127 | 0.163 | 90 |

TABLE 6
Dry powder characteristics and post reconstitution composition and
characteristics of mRNA-LNP dry powder with trehalose as excipient.

| | | | | Post Reconstitution Composition and Characteristics | | | | | | |
|---|---|---|---|---|---|---|---|---|---|---|
| Dry powder Characteristics | | | | | | | Trehalose/Lipid | | | |
| | Size | Reconstitution | Reconstituted | mRNA | Lipid | Trehalose % | ratio | Size | | Encapsulation |
| Yield % | (μm) | Solvent | (Yes/No) | (mg/ml) | (mg/ml) | (w/v) | (w/w) | (nm) | PDI | (%) |
| 82 | 4.7 | Water | Yes | 0.2 | 4.5 | 7 | 15.6 | 93 | 0.177 | 84 |
| 83 | 4.5 | Water | Yes | 0.2 | 4.5 | 5 | 11.1 | 93 | 0.166 | 86 |
| 75 | 3.5 | Water | Yes | 0.2 | 4.5 | 2.5 | 5.6 | 98 | 0.163 | 88 |
| 20 | 81.0 | Water | Yes | 0.2 | 4.5 | 1.25 | 2.8 | 134 | 0.118 | 91 |
| 0 | N/A | N/A | N/A | N/A | N/A | N/A | N/A | N/A | N/A | N/A |

TABLE 7
Dry powder characteristics and post reconstitution composition and characteristics of mRNA-LNP dry powder with
trehalose as excipient and reconstituted with trehalose added externally as part of the reconstituting solvent.

| Dry powder Characteristics | | | | Post Reconstitution Composition and Characteristics | | | | | | |
|---|---|---|---|---|---|---|---|---|---|---|
| | Size | Reconstitution | Reconstituted | mRNA | Lipid | Trehalose % | Trehalose/Lipid | Size | | Encapsulation |
| Yield % | (μm) | Solvent | (Yes/No) | (mg/ml) | (mg/ml) | (w/v) | ratio (w/w) | (nm) | PDI | (%) |
| 82 | 4.7 | Water | Yes | 0.2 | 4.5 | 1.25 | 2.8 | 134 | 0.118 | 91 |
| 83 | 4.5 | Trehalose | Yes | 0.2 | 4.5 | 5 | 11.1 | 136 | 0.123 | 71 |
| | | 3.75% | | | | | | | | |
| 75 | 3.5 | Trehalose | Yes | 0.2 | 4.5 | 7 | 15.6 | 134 | 0.132 | 71 |
| | | 5.75% | | | | | | | | |

### Example 3-Characterization of mRNA-LNP DPP

This experiment analyzed the physical characteristics of the mRNA-LNP based DPP generated in Example 1.

The size of mRNA-LNPs was measured using Zetasizer (Malvern). Pre- and post-spray drying encapsulation of the mRNA was determined using standard ribogreen assay. The size of the DPP particles was measured using Mastersizer (Malvern). The mRNA was extracted from the DPP and its integrity was determined using capillary electrophoresis (CE, Agilent). Optical microscope was used to identify the aggregates in the reconstituted dry powder.

As summarized in Table 8, lactose and xylitol did not yield any DPP. Although excellent dry powder characteristics were obtained for mannitol based DPP, it failed to reconstitute. Despite its higher dry powder size, sucrose based DPP reconstituted well at relative higher sucrose/lipid ratio. Not only the dry powder particle size was lower for trehalose based DPP but it also reconstituted well at relatively lower trehalose/lipid ratio as compared to sucrose based DPP. Thus, trehalose was identified as a viable sugar excipient for reconstitutable DPP.

TABLE 8
Dry powder product characterization summary

| Dry Powder | Powder | |
|---|---|---|
| Excipient | Characteristics | Reconstitution |
| Lactose | No | No |
| Xylitol | No | No |
| Mannitol | Yes | No |
| Sucrose | No | Yes |
| Trehalose | Yes | Yes |

### Example 4-Thermostability of Trehalose Based mRNA-LNP Dry Powder Product

This experiment analyzed the thermostability of the mRNA-LNP based DPP generated using trehalose as the sugar excipient (post reconstitution concentration 5% (w/v)) in terms of the change of particle size, polydispersity index (PDI), encapsulation efficiency (EE), and mRNA integrity. The LNP formulation used in this example contained CKK-E10 as the cationic lipid and the LNPs were loaded with mRNA encoding an influenza antigen.

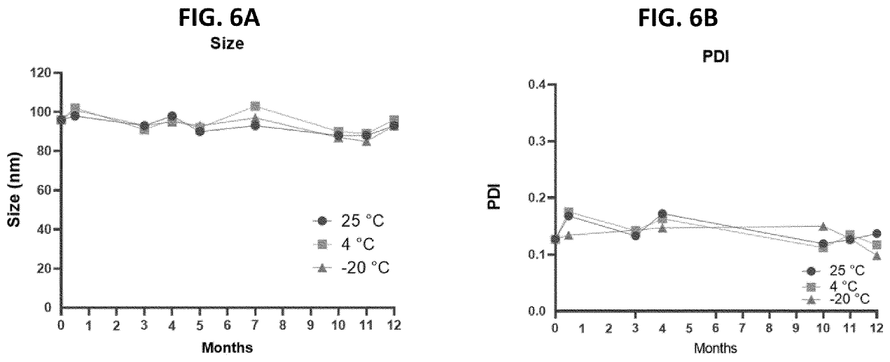

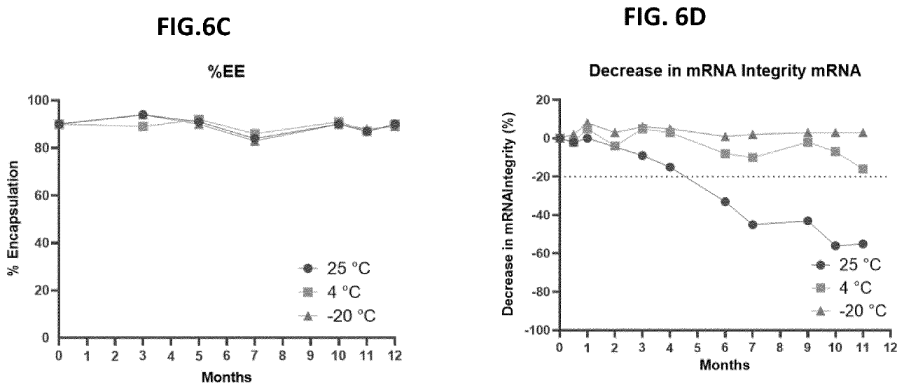

The particle size, DPI, encapsulation efficiency, and decrease in mRNA integrity were measured and calculated using known methods in the art. As shown in the FIG. 6A-FIG. 6D, no significant change in particle size (FIG. 6A), polydispersity index (FIG. 6B), encapsulation efficiency (FIG. 6C), and decrease in mRNA integrity (FIG. 6D) was observed after storage at 4° C. (square) or −20° C. (circle) for about a year.

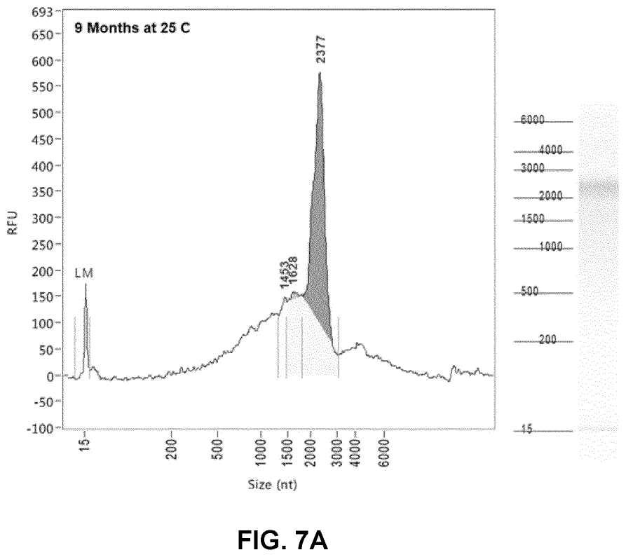

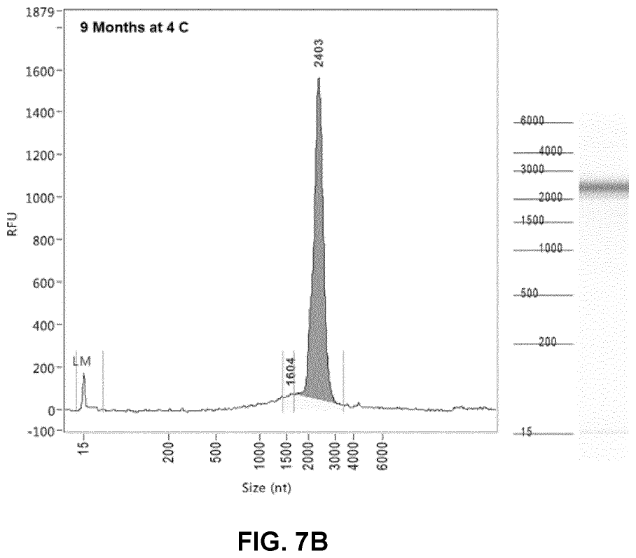

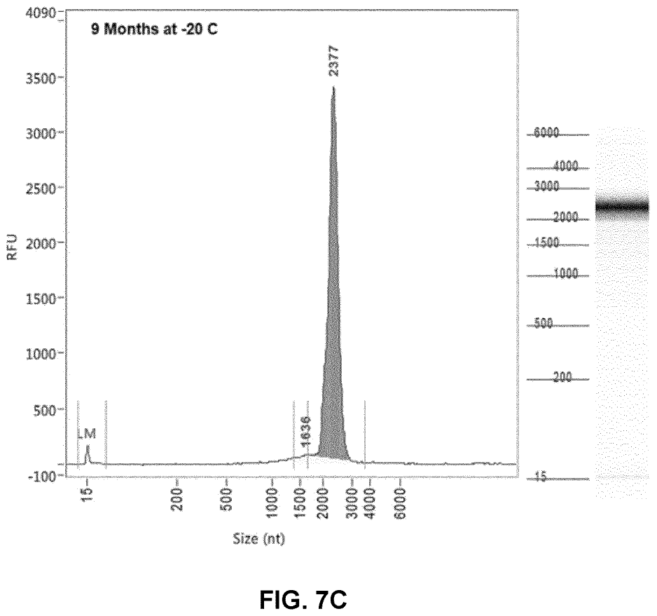

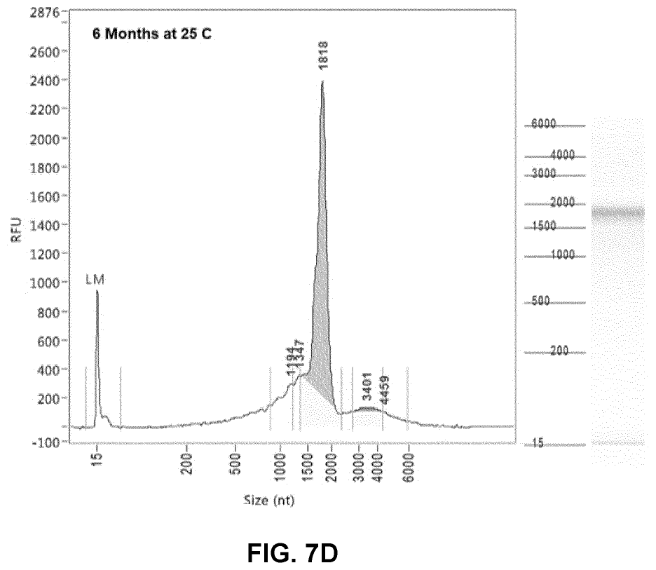

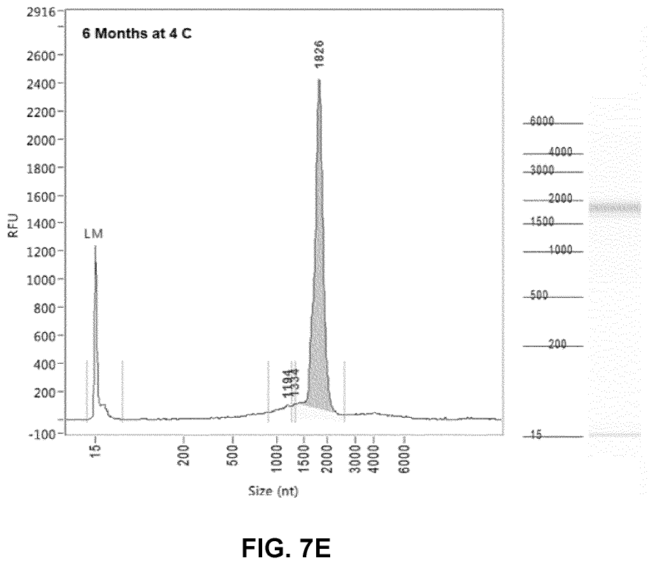

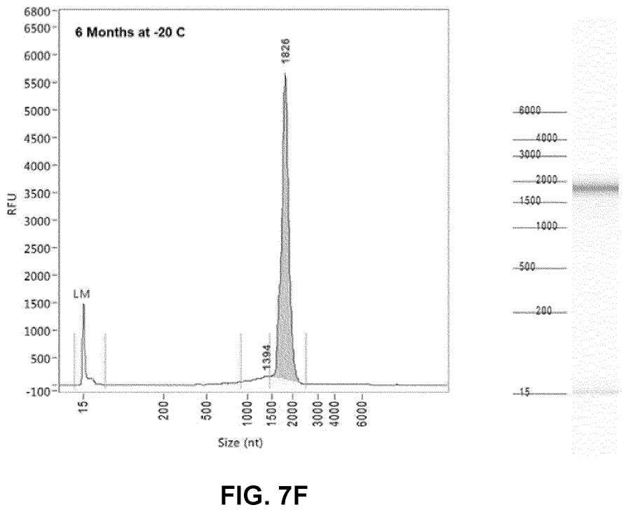

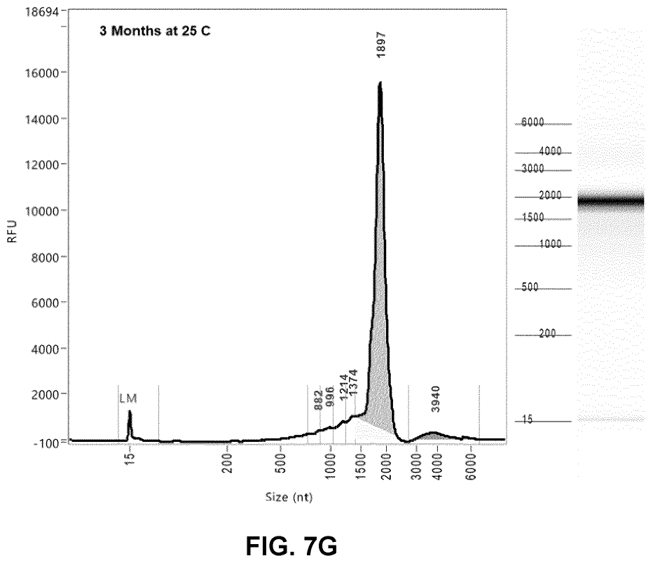

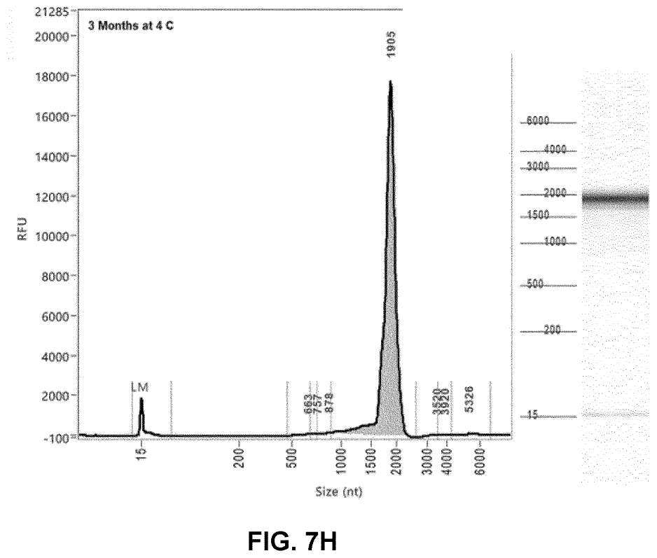

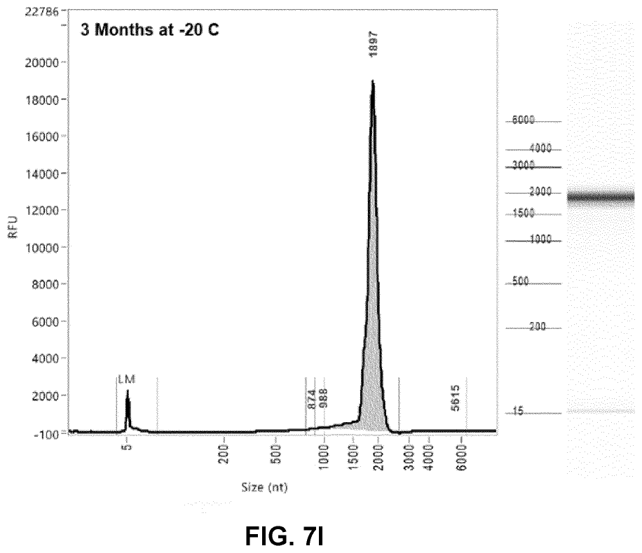

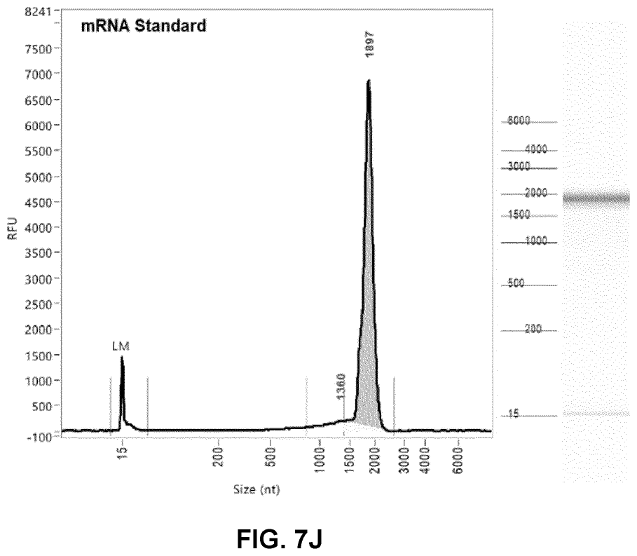

Additionally, the mRNA integrity after storage at different temperature (25° C., 4° C., or −20° C.) was assessed at different time point by extracting the mRNA from the LNP and analyzing on a fragment analyzer using capillary electrophoresis. As shown in FIG. 7A-FIG. 7I, no significant change in mRNA integrity was observed after storage at 4° C. and −20° C. for about 9 months, as compared to mRNA standard (FIG. 7J).

### Example 5-In Vivo Expression of Reconstituted mRNA-LNP DPP by Intravenous Delivery

In this example, the in vivo expression of the reconstituted mRNA-LNP DPP by intravenous delivery was evaluated in mice.

DPP was prepared by spray drying an mRNA-LNP formulation comprising mRNA encoding ornithine transcarbamylase (OTC) in the presence of trehalose as the sugar excipient. The DPP was reconstituted and administered intravenously in 6 to 8 weeks old CD-1 male mice at a dosage of 0.5 mg/kg. After 24 hours of administration, the protein expression of the OTC-coding mRNA was evaluated.

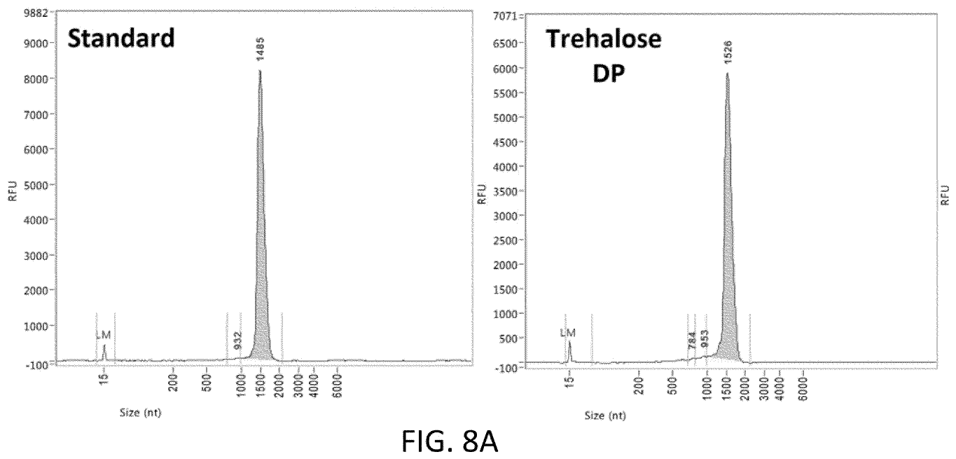

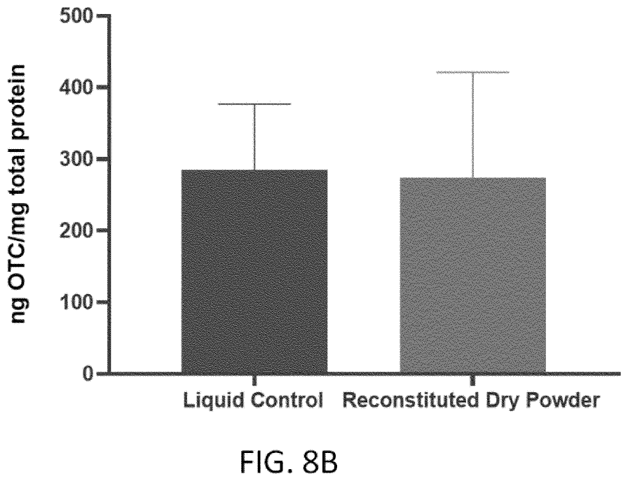

As shown in the FIG. 8A, post reconstitution of the trehalose dry powder (“Trehalose DP”) shows no change in mRNA integrity post spray drying as compared to a control mRNA (“Standard”). Moreover, as summarized in Table 9, the reconstituted trehalose dry powder has comparable characteristics as a liquid control which contains the same formulation without spray drying. As shown in the FIG. 8B, no significant difference in protein expression was detected between the liquid control and the reconstituted DPP.

TABLE 9
Characteristics of liquid control and reconstituted trehalose dry powder.

| | | Volume | mRNA | Sugar | Size | | EE |
|---|---|---|---|---|---|---|---|
| Drug Product | Solvent | (ml) | (mg/ml) | % (w/v) | (nm) | PDI | (%) |
| Liquid control | Water | 1 | 0.1 | 3 | 96 | 0.123 | 93 |
| Reconstituted | Water | 1 | 0.1 | 3 | 97 | 0.096 | 91 |
| trehalose dry powder | | | | | | | |

### Example 6-Various Dosage Form Presentations of mRNA-LNP DPP for Different Routes of Administration

This Example provides possible uses of mRNA-LNP dry powder, manufactured with sugar or sugar alcohol excipients, for treatment of one or more conditions.

The mRNA-LNP DPP will be compressed into various implants such as needle along with other excipients and can be directly injected using devices for different routes of administration such as oral, subcutaneous, and intramuscular. The mRNA-LNP DPP will be administered as powder or blended with excipients to manufacture granules, compressed into tablets that will be used for mucosal delivery via sublingual and buccal routes of administration. The additional excipients can be selected from commonly used compression agents such as microcrystalline cellulose (MCC), microfine cellulose (MFC), directly compressible starch (Sta-Rx 1500), dibasic calcium phosphate (DCP), spray dried lactose (SD lactose), anhydrous lactose (USP), fast Flo® lactose, spray-crystallized maltose and dextrose, crystalline sorbitol, mannitol, sucrose. Some of the commonly used granulating agents are sucrose, acacia, methyl cellulose, liquid glucose, tragacanth, ethyl cellulose, gelatin, hydroxy propyl methyl cellulose (HPMC), starch paste, hydroxy propyl cellulose, pregelatinized starch, sodium carboxy methyl cellulose, alginic acid, polyvinyl pyrrolidone (PVP), cellulose, polyethylene glycol (PEG), polyvinyl alcohols, polymethacrylates.

It is hypothesized that the tissues treated with the mRNA-LNP formulated with the discussed excipients will show high level of protein expression and result in significant alleviation of pathological phenotypes.

### Example 7-In Vivo Expression of Reconstituted mRNA-LNP DPP by Intramuscular Delivery

In this example, the in vivo expression of the reconstituted mRNA-LNP DPP by intramuscular delivery was evaluated in mice.

DPP was prepared by spray drying an mRNA-LNP formulation comprising mRNA encoding human erythropoietin (hEPO) in the presence of trehalose as the sugar excipient. The LNPs used in this example comprise cKK-E10 as the cationic lipid. The DPP was reconstituted and administered intramuscularly in mice at a dosage of 0.1 μg/animal. After 24 hours of administration, the protein expression of the hEPO-coding mRNA was evaluated.

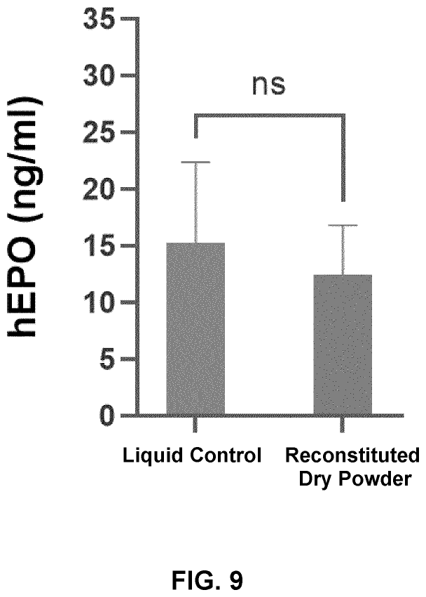

It was observed that mRNA integrity did not change from post reconstitution of the trehalose dry powder formulation to post spray drying as compared to a control mRNA (data not shown). Moreover, as summarized in Table 10, the reconstituted trehalose dry powder has comparable characteristics as a liquid control which contains the same formulation without spray drying. As shown in the FIG. 9, no significant difference in protein expression was observed between the liquid control and the reconstituted DPP.

TABLE 10
Contents post Reconstitution.

| | | | mRNA | Sugar | | | |
|---|---|---|---|---|---|---|---|
| | | Volume | (mg/ | % | Size | | EE |
| Drug Product | Solvent | (ml) | ml) | (w/v) | (nm) | PDI | (%) |
| Liquid Control | Water | 1 | 0.2 | 5 | 80 | 0.113 | 92 |
| Trehalose Dry | Water | 1 | 0.2 | 5 | 84 | 0.132 | 89 |
| powder (DP) | | | | | | | |

### Example 8-Improved Process for Storing mRNA-LNP Based DPP

This example describes an improved process for storing mRNA-LNP based DPP in a controlled environment.

Briefly, tubes containing DPPs were stored in vacuum-sealed bags, which were then kept in a chamber containing desiccant and filled with nitrogen gas. Desiccant absorbs the moisture in the chamber and nitrogen gas provides an inert overlay, which created a control, low humidity storage condition.

Three different DPPs were used for long-term thermostability study at 2-8° C. (e.g., 4° C.) with this improved process, each was prepared by encapsulating a different mRNA in LNPs containing cKK-E10 as the cationic lipid and spray-dried in the presence of trehalose as the excipient. The mRNAs used in this study were mRNA encoding hEPO, mRNA encoding an influenza antigen (mono-Flu), and mRNA encoding an antigen from respiratory syncytial virus (RSV). Samples of each DPP were removed from the tube and reconstituted for thermostability testing, including change in particle size, PDI, encapsulation efficiency, and mRNA integrity, at different time point. The DPP prepared with mRNA encoding hEPO was also tested for the mRNA expression at different time points.

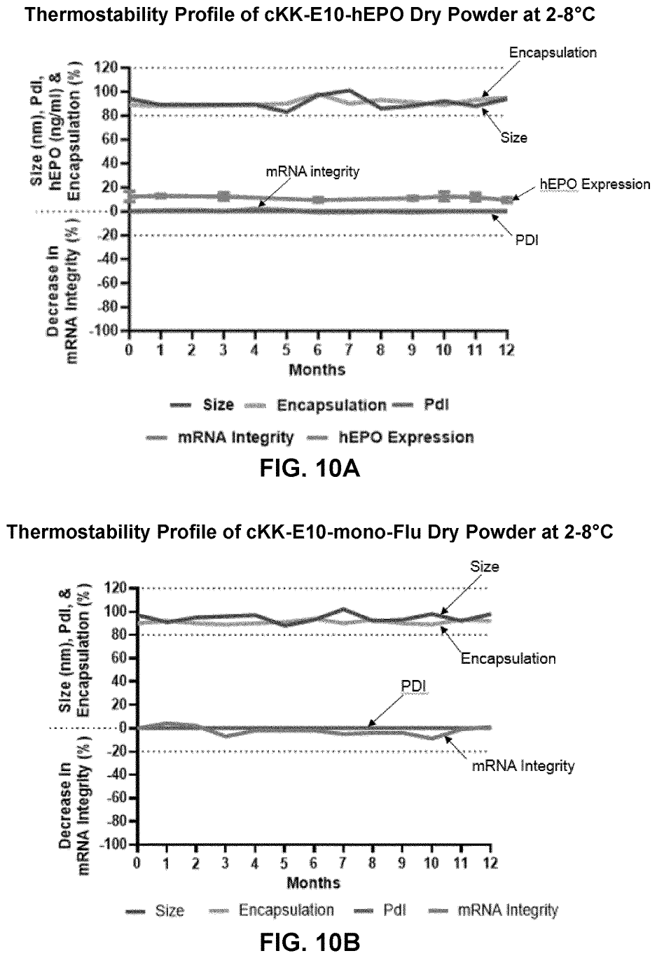

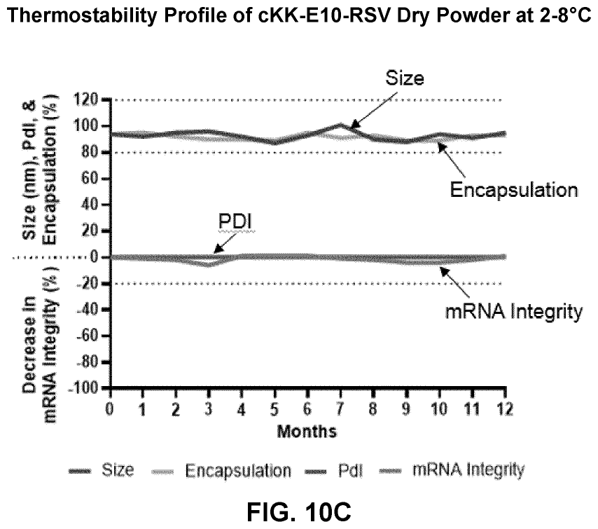

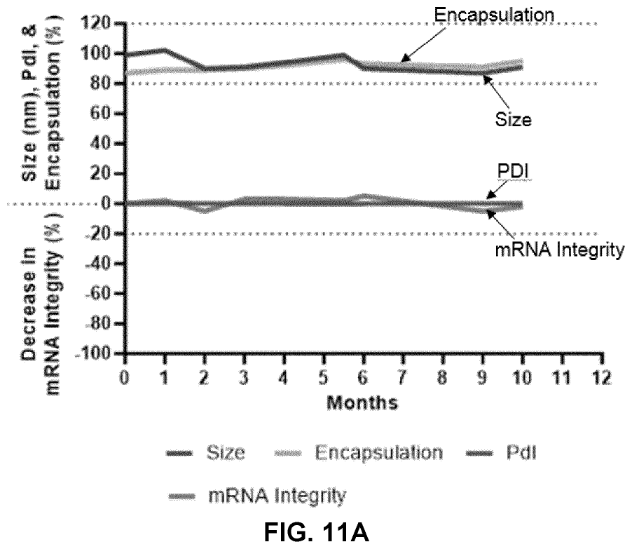

As shown in FIG. 10A-FIG. 10C, there was no significant change in particle size, PDI, encapsulation efficiency, and mRNA integrity over the course of 12 months for the three different DPPs tested when stored at 2-8° C. using this improved process. As shown in FIG. 10A, there was also no significant change in the hEPO expression over the course of 12 months.

Another DPP prepared with 4 different mRNAs, each encoding a different influenza antigen (QIV-Flu), was also used for long-term thermostability study at 2-8° C. (e.g., 4° C.) with this improved process. This DPP was prepared by encapsulating the 4 different mRNAs in LNPs containing GL-HEPES-E3-E12-DS-4-E10 as the cationic lipid and spray-dried in the presence of trehalose as the excipient. Samples of the DPP were removed from the tube and reconstituted for thermostability testing, including change in particle size, PDI, encapsulation efficiency, and mRNA integrity, at different time point.

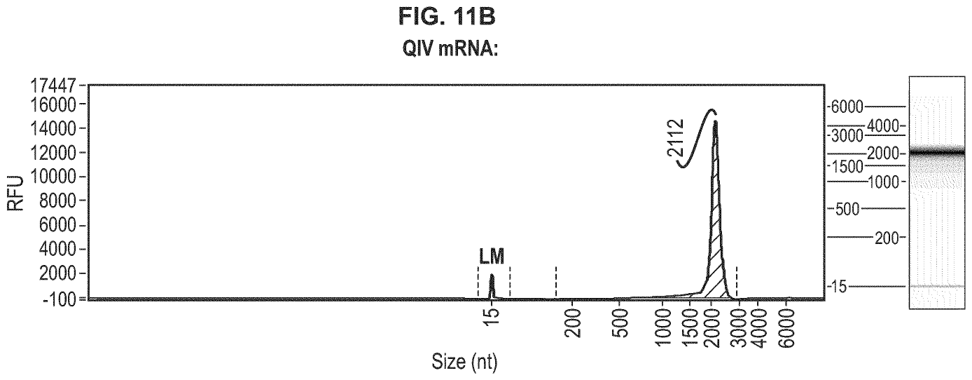

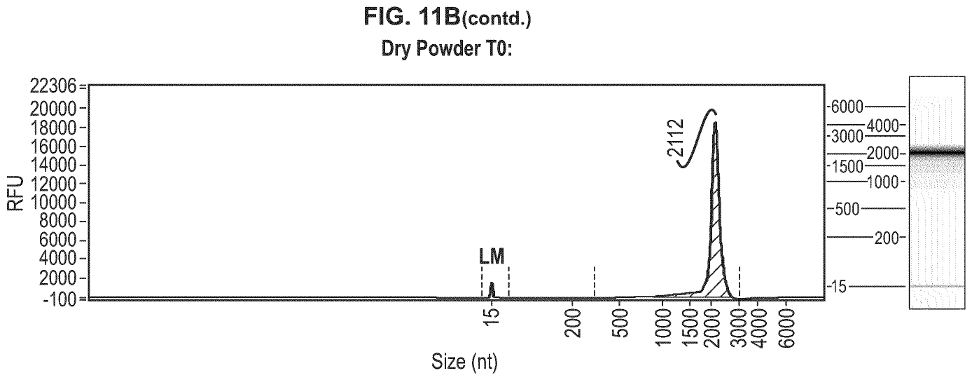

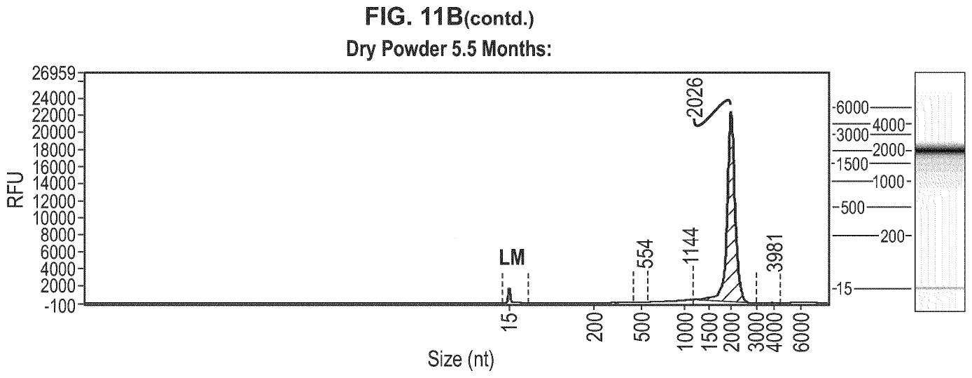

As shown in FIG. 11A, there was no significant change in particle size, PDI, encapsulation efficiency, and mRNA integrity over the course of 10 months when the DPP was stored at 2-8° C. using this improved process. The capillary electrophoresis profiles of the QIV-Flu mRNA, the mRNA extracted from the reconstituted DPP at TO, and the mRNA extracted from the reconstituted DPP after storage at 2-8° C. for 5.5 months shown in FIG. 11B further confirm that no significant change in mRNA integrity occurred after storage at 2-8° C. for more than 5 months.

We claim:

1. A dry powder formulation for reconstitution comprising:
(a) an excipient that is sucrose or trehalose; and
(b) a lipid nanoparticle (LNP) comprising a messenger RNA (mRNA) encapsulated by one or more lipids;
wherein the excipient of (a) and total lipids in the LNP of (b) have a weight ratio of at least about 5.

2. The dry powder formulation of claim 1, wherein the excipient is sucrose.

3. The dry powder formulation of claim 1, wherein the excipient is trehalose.

4. The dry powder formulation of any one of claims 1-3, wherein the weight ratio of the excipient to the total lipids in the LNP is at least about 5.6, at least about 11, or at least about 15.

5. The dry powder formulation of claim 4, wherein the weight ratio of the excipient to the total lipids is at least about 11.1 or at least about 15.6.

6. The dry powder formulation of any one of claims 1-5, wherein the LNP comprises one or more cationic lipids, one or more non-cationic lipids, and one or more PEG-modified lipids.

7. A dry powder formulation for reconstitution comprising
(a) an excipient that is a sugar or sugar alcohol; and
(b) a lipid nanoparticle (LNP) comprising a messenger RNA (mRNA) encapsulated by one or more lipids;
wherein weight ratio of the excipient of (a) to total lipids in the LNP of (b) is at least about 5.

8. The dry powder formulation of claim 7, wherein the sugar or sugar alcohol is sucrose, mannitol, xylitol, lactose, or trehalose.

9. The dry powder formulation of claim 7, wherein the sugar or sugar alcohol is trehalose.

10. The dry powder formulation of claim 7, wherein the sugar or sugar alcohol is sucrose.

11. The dry powder formulation of any one of claims 7-10, wherein the weight ratio of the sugar or sugar alcohol to the total lipids in the LNP is at least about 5.6, at least about 11, or at least about 15.

12. The dry powder formulation of claim 7, wherein the weight ratio of the sugar or sugar alcohol to the total lipids in the LNP is at least about 11.1 or at least about 15.6.

13. The dry powder formulation of any one of claims 7-12, wherein the LNP comprises one or more cationic lipids, one or more non-cationic lipids, and one or more PEG-modified lipids.

14. The dry powder formulation of any one of claims 1-13, wherein the particle size of the reconstituted dry powder formulation is less than about 120 nm.

15. The dry powder formulation of any one of claims 1-14, wherein the encapsulation rate is greater than about 60%.

16. The dry powder formulation of any one of claims 1-15, wherein the particle size of the dry powder is less than about 5 μm.

17. The dry powder formulation of any one of claims 1-16, wherein the dry powder formulation is stable after prolonged storage, for example, after prolonged storage at 2-8° C. (e.g., at 4° C.).

18. The dry powder formulation of claim 17, wherein the mRNA maintains an integrity of about 80% or greater after storage, e.g., after storage at 2-8° C. (e.g., 4° C.) for at least about six months.

19. The dry powder formulation of claim 18, wherein the mRNA maintains an integrity of about 80% or greater, e.g., after storage at 2-8° C. (e.g., 4° C.) for up to about 1 year.

20. The dry powder formulation of claim 19, wherein the mRNA maintains an integrity of about 80% or greater, optionally where mRNA integrity is measured after storage at 2-8° C. (e.g., 4° C.) for at least 1 year.

21. The dry powder formulation of any one of claims 1-20, wherein the mRNA encodes a therapeutic protein.

22. The dry powder formulation of any one of claims 1-21, wherein the mRNA encodes an antigen.

23. The dry powder formulation of any one of claims 1-22, wherein the formulation is suitable for a vaccine.

24. The dry powder formulation of any one of claims 1-23, wherein the formulation is suitable for parenteral delivery once reconstituted.

25. The dry powder formulation of claim 24, wherein the formulation is suitable for intramuscular, intravenous, or subcutaneous delivery once reconstituted.

26. The dry powder formulation of any one of claims 1-25, wherein the formulation is suitable for mucosal delivery once reconstituted.

27. The dry powder formulation of claim 26, wherein the formulation is suitable for oral, sublingual, or intranasal delivery once reconstituted.

28. A method of delivering a mRNA in vivo, the method comprising administering to a subject in need thereof a reconstituted form of the dry powder formulation of any one of claims 1-27.

29. A method of treating a disease or disorder in a subject, the method comprising administering to the subject a reconstituted form of the dry powder formulation of any one of claims 1-27.

30. The method of claim 28 or 29, wherein the reconstituted form of the dry powder formulation is administered subcutaneously, intravenously, or intramuscularly.

31. A method of preparing a dry powder formulation for reconstitution, the method comprising:
(a) combining a first mixture comprising lipid nanoparticles (LNPs) and an ethanolic solution, thereby obtaining a second mixture, wherein the LNPs comprise a messenger RNA (mRNA) encapsulated by one or more lipids, and wherein the ethanolic solution comprises an excipient that is sucrose or trehalose at a concentration of about 1-11% w/v, and
(b) spray-drying the second mixture, thereby obtaining the dry powder formulation.

32. A method of preparing a reconstituted dry powder formulation, the method comprising:
(a) providing a dry powder formulation prepared according to the method of claim 31; and
(b) reconstituting the dry powder formulation in water or buffer, thereby obtaining the reconstituted dry powder formulation.

33. The method of claim 32, wherein the LNPs in the reconstituted dry powder formulation have a diameter of about 100 nm.

34. The method of any one of claims 31-33, wherein sucrose or trehalose and total lipids in the LNPs have a weight ratio of at least about 5, at least about 11, or at least about 15.

35. The method of any one of claims 31-34, wherein the concentration of sucrose or trehalose is greater than about 2% (w/v), greater than about 5% (w/v), or greater than about 7% (w/v) post-reconstitution.

36. The method of any one of claims 31-35, wherein the concentration of sucrose or trehalose is from about 2% (w/v) to about 10% (w/v) post-reconstitution.

37. The method of any one of claims 32-36, wherein the particle size of the reconstituted dry-powder formulation is less than about 120 nm.

38. The method of any one of claims 32-37, wherein the reconstituted LNP particle size is between about 80 nm and about 120 nm.

39. The method of any one of claims 32-38, wherein the reconstituted LNP particle size is between about 80 nm and about 115 nm.
